(19) United States
(12) Patent Application Publication
(10) Pub. No.: US 2026/0351355 A1
(43) Pub. Date: Nov. 5, 2026

(54) DRUG DISCOVERY ECOSYSTEM WITH ARTIFICIAL INTELLIGENCE AGENT DESIGN INFRASTRUCTURE FOR DYNAMIC TOOL SELECTION AND ORCHESTRATION

(71) Applicants: Genentech, Inc., South San Francisco, CA (US); F. Hoffmann-La Roche AG, Basel (CH); Hoffmann-La Roche Inc., Little Falls, NJ (US); Roche Diagnostics GmbH, Penzberg (DE); Roche Glycart AG, Sclieren (CH); Roche Polska Sp. z o.o., Warsaw (PL)

(72) Inventors: Jorge Oswaldo GOMEZ MUÑOZ, Poznan (PL); Colin Andreas GRAMBOW, San Francisco, CA (US); Taylor Mackay JOREN, La Jolla, CA (US); Malamati KOLETOU, Kilchberg (CH); Krystian KOZIEL, Warsaw (PL); John Andrew LASH, Oakland, CA (US); Sidney Lyayuga LISANZA, Brooklyn, NY (US); Derek MA, New York, NY (US); Le MU, Munich (DE); Witold Maria PRZECIECHOWSKI, San Carlos, CA (US); Lukasz RAKOCZY, Poznan (PL); Michael Robert REUTLINGER, Freiburg im Breisgau (DE); Stephen Robert RA, Brooklyn, NY (US); Alin Alexandru SPINU, Buchares (RO); Zichen WANG, Jersey City, NJ (US); Andrew Martin WATKINS, San Francisco, CA (US); Karina ZADOROZHNY, Rockville, MD (US); Yun Yvonna LI, Basel (CH); Josep ARUS POUS, Sabadell (SP); Anna Katharina BAUER-MEHREN, Gauting (DE); Daniel BUTNARU, Bottmingen (CH); Christophe Didier Fernand CHABBERT, Neuheim (CH); An CHEN, Millburn, NJ (US); Shriram CHENNAKESAVALU, San Francisco, CA (US); Henri Vincent DWYER, Brooklyn, NY (US); Nathan Christopher FREY, New York, NY, NY (US)

(21) Appl. No.: 19/730,236

(22) Filed: Jul. 2, 2026

Related U.S. Application Data
(63) Continuation of application No. PCT/US2026/013034, filed on Jan. 29, 2026.
(60) Provisional application No. 63/751,204, filed on Jan. 29, 2025.
(60) Provisional application No. 63/751,128, filed on Jan. 29, 2025.
(60) Provisional application No. 63/756,682, filed on Feb. 10, 2025.
(60) Provisional application No. 63/901,426, filed on Oct. 17, 2025.
(60) Provisional application No. 63/906,972, filed on Oct. 28, 2025.

(30) Foreign Application Priority Data
Jan. 26, 2026 (GR) 20260100065

(51) Int. Cl. G16C 20/50; G16C 20/70

(52) U.S. Cl. CPC G16C 20/50; G16C 20/70

(57) ABSTRACT

A drug discovery ecosystem may receive one or more user inputs configuring an artificial intelligence (AI) agent for orchestrating a drug discovery workflow applying one or more drug discovery tools. The artificial intelligence (AI) agent may be created in accordance with the one or more user inputs. The artificial intelligence (AI) agent is served to one or more users as part a selection of artificial intelligence (AI) agents. One or more user inputs selecting the artificial intelligence (AI) agent may be received. The artificial intelligence (AI) agent may be executed to orchestrate the drug discovery workflow in response to the one or more user inputs selecting the artificial intelligence (AI) agent. Related systems, computer-implemented methods, and computer program products are provided.

### CROSS REFERENCE TO RELATED APPLICATIONS

This application claims priority to U.S. Provisional Application No. 63/751,204, entitled “INTENTION-AWARE LARGE LANGUAGE MODEL AGENT FOR DRUG DISCOVERY” and filed on Jan. 29, 2025, U.S. Provisional Application No. 63/751,128, entitled “LAB-IN-THE-LOOP THERAPEUTIC PROTEIN DESIGN WITH DEEP LEARNING” and filed on Jan. 29, 2025, U.S. Provisional Application No. 63/756,682, entitled “INTENTION-AWARE LARGE LANGUAGE MODEL AGENT FOR DRUG DISCOVERY” and filed on Feb. 10, 2025, U.S. Provisional Application No. 63/901,426, entitled “INTENTION-AWARE LARGE LANGUAGE MODEL AGENT FOR DRUG DISCOVERY” and filed on Oct. 17, 2025, U.S. Provisional Application No. 63/906,972, entitled “LAB-IN-THE-LOOP THERAPEUTIC PROTEIN DESIGN WITH DEEP LEARNING” and filed on Oct. 28, 2025, Greek Provisional Application No. 20260100065, entitled “A COMPREHENSIVE ML PLATFORM FOR AI MODEL DEPLOYMENT IN DRUG DISCOVERY” and filed on Jan. 26, 2026, the disclosures of which are incorporated herein by reference in their entireties.

### TECHNICAL FIELD

The subject matter described herein relates generally to molecular design and more specifically to a drug discovery ecosystem with infrastructure to support the design and deployment of artificial intelligence (AI) agents for dynamic selection and orchestration of drug discovery tools.

### INTRODUCTION

A molecule is a group of two more atoms held together by chemical bonds. Molecules form the smallest identifiable unit into which a pure substance can be divided while still retaining the composition and chemical properties of that substance. Various properties of a molecule, including its ability to function as a therapeutic, may be contingent upon the conformation (or three-dimensional structure) of the molecule. One example of a molecule is a small molecule, which is a low-weight compound having a molecular weight between approximately 100 Daltons and 1000 Daltons. Small molecule therapeutics, which modulate biochemical processes to diagnose, treat, and prevent a gamut of illnesses, have been a cornerstone in modern pharmacology due to a number of compelling advantages. For example, small molecule drugs are capable of penetrating cell membranes to reach intracellular targets. Moreover, small molecule drugs are adaptable to a wide variety of therapeutic applications. For instance, a small molecule drug may be formulated as pills and capsules, intravenous or subcutaneous injectables, inhalational medicines, or suppositories. The development of the small molecule drug may further extend to tailoring various pharmacokinetic properties including liberation, absorption, distribution, metabolism, potency, efficacy, phenotypic effects, and excretion.

By contrast, large molecules (also known as biopharmaceuticals, biologicals, or biologics) can range between approximately 3000 Daltons and 150,000 Daltons in molecular weight. Large molecule drugs are often derivatives of natural human proteins, which modulate many essential cellular functions such as enzymatic reactions, transport of molecules, regulation and execution of a number of biological pathways, cell growth, proliferation, nutrient uptake, morphology, motility, intercellular communication, and/or the like. It is common for a single large molecule to have more than 1,300 amino acid residues, which are linked by peptide bonds to form one or more polypeptide. Due to their size and complexity, large molecule drugs are recombinantly produced by engineered cells instead of being chemically synthesized like the majority of small molecule drugs. Moreover, large molecule therapeutics are usually delivered through injection or infusion due to the ineffectiveness of oral administration. The development of a large molecule drug may entail designing one or more sequences of amino acid residues capable of binding to a target (e.g., a protein, a nucleic acid, and/or the like) with sufficient specificity and absent undesirable traits such as immunogenicity, self-association, instability, and/or the like.

### SUMMARY

Systems, methods, and articles of manufacture, including computer program products, are provided for a drug discovery ecosystem with infrastructure to support the design and deployment of artificial intelligence (AI) agents for dynamic selection and orchestration of drug discovery tools.

In one aspect, there is provided a system for a drug discovery ecosystem. The system may include at least one data processor and at least one memory. The at least one memory may store instructions that result in operations when executed by the at least one data processor. The operations may include: receiving one or more user inputs configuring an artificial intelligence (AI) agent for orchestrating a drug discovery workflow applying one or more drug discovery tools; creating the artificial intelligence (AI) agent in accordance with the one or more user inputs; serving the artificial intelligence (AI) agent to one or more users as part a selection of artificial intelligence (AI) agents; receiving one or more user inputs selecting the artificial intelligence (AI) agent; and executing the artificial intelligence (AI) agent to orchestrate the drug discovery workflow in response to the one or more user inputs selecting the artificial intelligence (AI) agent.

In another aspect, there is provided a computer-implemented method for a drug discovery ecosystem. The method may include: receiving one or more user inputs configuring an artificial intelligence (AI) agent for orchestrating a drug discovery workflow applying one or more drug discovery tools; creating the artificial intelligence (AI) agent in accordance with the one or more user inputs; serving the artificial intelligence (AI) agent to one or more users as part a selection of artificial intelligence (AI) agents; receiving one or more user inputs selecting the artificial intelligence (AI) agent; and executing the artificial intelligence (AI) agent to orchestrate the drug discovery workflow in response to the one or more user inputs selecting the artificial intelligence (AI) agent.

In another aspect, there is provided a computer program product for a drug discovery ecosystem. The computer program product may include a non-transitory computer readable medium storing instructions that result in operations when executed by at least one data processor. The operations may include: receiving one or more user inputs configuring an artificial intelligence (AI) agent for orchestrating a drug discovery workflow applying one or more drug discovery tools; creating the artificial intelligence (AI) agent in accordance with the one or more user inputs; serving the artificial intelligence (AI) agent to one or more users as part a selection of artificial intelligence (AI) agents; receiving one or more user inputs selecting the artificial intelligence (AI) agent; and executing the artificial intelligence (AI) agent to orchestrate the drug discovery workflow in response to the one or more user inputs selecting the artificial intelligence (AI) agent.

In another aspect, there is provided a system for a drug discovery ecosystem. The system may include at least one data processor and at least one memory. The at least one memory may store instructions that result in operations when executed by the at least one data processor. The operations may include: receiving one or more user inputs invoking a drug discovery workflow applying one or more drug discovery tools; registering, with a molecule registration system, a molecule design generated by applying a drug discovery tool in the drug discovery workflow; applying a subsequent drug discovery tool in the drug discovery workflow by at least providing, as input to the subsequent drug discovery tool, a unique molecule identifier assigned to the molecule design by registering with the molecule registration system; and retrieving, by the subsequent drug discovery tool, data associated with the molecule design from the molecule registration system, wherein the data associated with the molecule design is retrieved from the molecule registration system based at least on the unique molecule identifier of the molecule design, and wherein the subsequent drug discovery tool determines an output based at least on the data retrieved from the molecule registration system.

In another aspect, there is provided a computer-implemented method for a drug discovery ecosystem. The method may include: receiving one or more user inputs invoking a drug discovery workflow applying one or more drug discovery tools; registering, with a molecule registration system, a molecule design generated by applying a drug discovery tool in the drug discovery workflow; applying a subsequent drug discovery tool in the drug discovery workflow by at least providing, as input to the subsequent drug discovery tool, a unique molecule identifier assigned to the molecule design by registering with the molecule registration system; and retrieving, by the subsequent drug discovery tool, data associated with the molecule design from the molecule registration system, wherein the data associated with the molecule design is retrieved from the molecule registration system based at least on the unique molecule identifier of the molecule design, and wherein the subsequent drug discovery tool determines an output based at least on the data retrieved from the molecule registration system.

In another aspect, there is provided a computer program product for a drug discovery ecosystem. The computer program product may include a non-transitory computer readable medium storing instructions that result in operations when executed by at least one data processor. The operations may include: receiving one or more user inputs invoking a drug discovery workflow applying one or more drug discovery tools; registering, with a molecule registration system, a molecule design generated by applying a drug discovery tool in the drug discovery workflow; applying a subsequent drug discovery tool in the drug discovery workflow by at least providing, as input to the subsequent drug discovery tool, a unique molecule identifier assigned to the molecule design by registering with the molecule registration system; and retrieving, by the subsequent drug discovery tool, data associated with the molecule design from the molecule registration system, wherein the data associated with the molecule design is retrieved from the molecule registration system based at least on the unique molecule identifier of the molecule design, and wherein the subsequent drug discovery tool determines an output based at least on the data retrieved from the molecule registration system.

In another aspect, there is provided a system for a drug discovery ecosystem. The system may include at least one data processor and at least one memory. The at least one memory may store instructions that result in operations when executed by the at least one data processor. The operations may include: receiving, at a molecule registration system, data associated with a molecule design; determining a molecule identifier to include a concept identifier of a concept of the molecule design, wherein the concept of the molecule design corresponds to a conceptualized version of a corresponding molecule; and storing, in a repository, at least a portion of the data associated with the molecule design, wherein the data associated with the molecule design is stored along with the molecule identifier.

In another aspect, there is provided a computer-implemented method for a drug discovery ecosystem. The method may include: receiving, at a molecule registration system, data associated with a molecule design; determining a molecule identifier to include a concept identifier of a concept of the molecule design, wherein the concept of the molecule design corresponds to a conceptualized version of a corresponding molecule; and storing, in a repository, at least a portion of the data associated with the molecule design, wherein the data associated with the molecule design is stored along with the molecule identifier.

In another aspect, there is provided a computer program product for a drug discovery ecosystem. The computer program product may include a non-transitory computer readable medium storing instructions that result in operations when executed by at least one data processor. The operations may include: receiving, at a molecule registration system, data associated with a molecule design; determining a molecule identifier to include a concept identifier of a concept of the molecule design, wherein the concept of the molecule design corresponds to a conceptualized version of a corresponding molecule; and storing, in a repository, at least a portion of the data associated with the molecule design, wherein the data associated with the molecule design is stored along with the molecule identifier.

In another aspect, there is provided a system for a drug discovery ecosystem. The system may include at least one data processor and at least one memory. The at least one memory may store instructions that result in operations when executed by the at least one data processor. The operations may include: receiving one or more user inputs invoking a drug discovery workflow applying one or more drug discovery tools; registering, with a molecule registration system, a molecule design generated by applying a drug discovery tool in the drug discovery workflow; applying a subsequent drug discovery tool in the drug discovery workflow by at least providing, as input to the subsequent drug discovery tool, a unique molecule identifier assigned to the molecule design by registering with the molecule registration system; and retrieving, by the subsequent drug discovery tool, data associated with the molecule design from the molecule registration system, wherein the data associated with the molecule design is retrieved from the molecule registration system based at least on the unique molecule identifier of the molecule design, and wherein the subsequent drug discovery tool determines an output based at least on the data retrieved from the molecule registration system.

In another aspect, there is provided a computer-implemented method for a drug discovery ecosystem. The method may include: receiving one or more user inputs invoking a drug discovery workflow applying one or more drug discovery tools; registering, with a molecule registration system, a molecule design generated by applying a drug discovery tool in the drug discovery workflow; applying a subsequent drug discovery tool in the drug discovery workflow by at least providing, as input to the subsequent drug discovery tool, a unique molecule identifier assigned to the molecule design by registering with the molecule registration system; and retrieving, by the subsequent drug discovery tool, data associated with the molecule design from the molecule registration system, wherein the data associated with the molecule design is retrieved from the molecule registration system based at least on the unique molecule identifier of the molecule design, and wherein the subsequent drug discovery tool determines an output based at least on the data retrieved from the molecule registration system.

In another aspect, there is provided a computer program product for a drug discovery ecosystem. The computer program product may include a non-transitory computer readable medium storing instructions that result in operations when executed by at least one data processor. The operations may include: receiving one or more user inputs invoking a drug discovery workflow applying one or more drug discovery tools; registering, with a molecule registration system, a molecule design generated by applying a drug discovery tool in the drug discovery workflow; applying a subsequent drug discovery tool in the drug discovery workflow by at least providing, as input to the subsequent drug discovery tool, a unique molecule identifier assigned to the molecule design by registering with the molecule registration system; and retrieving, by the subsequent drug discovery tool, data associated with the molecule design from the molecule registration system, wherein the data associated with the molecule design is retrieved from the molecule registration system based at least on the unique molecule identifier of the molecule design, and wherein the subsequent drug discovery tool determines an output based at least on the data retrieved from the molecule registration system.

In another aspect, there is provided a system for a drug discovery ecosystem. The system may include at least one data processor and at least one memory. The at least one memory may store instructions that result in operations when executed by the at least one data processor. The operations may include: applying a molecule design computation model to generate a molecule design; applying a property computation model to determine one or more properties of interest present in the molecule design, wherein the property computation model is trained to determine the one or more properties of interest based at least on a contextual information, and wherein the contextual information includes a description of one or more controlled measurement parameters of an experimental environment in which the one or more properties of interest are measured; and applying a selection computation model to select, based at least on the one or more properties of interest, the molecule design for experimental validation.

In another aspect, there is provided a computer-implemented method for a drug discovery ecosystem. The method may include: applying a molecule design computation model to generate a molecule design; applying a property computation model to determine one or more properties of interest present in the molecule design, wherein the property computation model is trained to determine the one or more properties of interest based at least on a contextual information, and wherein the contextual information includes a description of one or more controlled measurement parameters of an experimental environment in which the one or more properties of interest are measured; and applying a selection computation model to select, based at least on the one or more properties of interest, the molecule design for experimental validation.

In another aspect, there is provided a computer program product for a drug discovery ecosystem. The computer program product may include a non-transitory computer readable medium storing instructions that result in operations when executed by at least one data processor. The operations may include: applying a molecule design computation model to generate a molecule design; applying a property computation model to determine one or more properties of interest present in the molecule design, wherein the property computation model is trained to determine the one or more properties of interest based at least on a contextual information, and wherein the contextual information includes a description of one or more controlled measurement parameters of an experimental environment in which the one or more properties of interest are measured; and applying a selection computation model to select, based at least on the one or more properties of interest, the molecule design for experimental validation.

In some variations, one or more features disclosed herein including the following features can optionally be included in any feasible combination.

In some variations, the one or more drug discovery tools include a molecule design computation model trained to generate one or more molecule designs.

In some variations, the one or more drug discovery tools include a selection computation model trained to rank two or more molecule designs based on one or more properties of interest.

In some variations, the one or more drug discovery tools include a property computation model trained to determine one or more properties of interest present in a molecule design.

In some variations, one or more user inputs configuring a drug discovery tool are received. The drug discovery tool is deployed to a tool server to provide the drug discovery tool to the one or more users as part of a selection menu of drug discovery tools.

In some variations, the drug discovery tool is identified for inclusion in the drug discovery workflow.

In some variations, one or more user inputs selecting the drug discovery tool for inclusion in the drug discovery workflow are received. The drug discovery tool is identified for inclusion in the drug discovery workflow based at least on the one or more user inputs selecting the drug discovery tool.

In some variations, the drug discovery tool is included in the drug discovery workflow for application by the artificial intelligence (AI) agent orchestrating the drug discovery workflow.

In some variations, the artificial intelligence (AI) agent orchestrates the drug discovery workflow by at least determining a sequence in which to apply the one or more drug discovery tools included in the drug discovery workflow.

In some variations, the sequence in which the one or more drug discovery tools is applied comprises a predetermined sequence.

In some variations, the sequence in which the one or more drug discovery tools is applied comprises a dynamic sequence determined by the artificial intelligence (AI) agent.

In some variations, the artificial intelligence (AI) agent determines the sequence of applying the one or more drug discovery tools based at least on the one or more drug discovery tools included in the drug discovery workflow.

In some variations, the artificial intelligence (AI) agent determines the sequence of applying the one or more drug discovery tools based at least on one or more outputs resulting from an application of at least one drug discovery tool in the drug discovery workflow.

In some variations, the artificial intelligence (AI) agent determines the sequence of applying the one or more drug discovery tools based at least on one or more inputs received from an end user invoking the drug discovery workflow.

In some variations, at least a portion of the one or more inputs received from the end user comprise a selection of one or more preconfigured inputs provided to the end user.

In some variations, the artificial intelligence (AI) agent determines the sequence of applying the one or more drug discovery tools by at least synthesizing a set of reference intention-action pairs, identifying, within the set of reference intention-action pairs, one or more intentions that are a semantic match to one or more inputs received from an end user invoking the drug discovery workflow, determining a trajectory of actions based at least on one or more intention-action pairs corresponding to the one or more intentions that are a semantic match to the one or more inputs from the end user, and determining, based at least on the trajectory actions, the sequence of applying the one or more drug discovery tools.

In some variations, the synthesizing the set of reference intention-action pairs includes determining, based at least on a set of available drug discovery tools, a set of valid action chains in which an upstream drug discovery tool's output datatype corresponds to a downstream drug discovery tool's input datatype, applying a language model to determine, for each valid action chain, a corresponding intention, and generating each intention-action pair in the set of reference intention-action pairs to include a valid action chain and the corresponding intention.

In some variations, the one or more user inputs configure the drug discovery tool by at least configuring one or more capabilities of the drug discovery tool.

In some variations, the one or more user inputs configure the drug discovery tool by at least implementing a tool server for serving the drug discovery tool.

In some variations, the artificial intelligence (AI) agent orchestrates the drug discovery workflow by at least selecting, based at least on one or more inputs from an end user invoking the drug discovery workflow, a drug discovery tool, generating, based at least on one or more required inputs of the drug discovery tool, a prompt soliciting one or more additional inputs from the end user, and applying the drug discovery tool with the one or more additional inputs from the end user.

In some variations, the artificial intelligence (AI) agent continues orchestrating the drug discovery workflow by at least identifying a subsequent drug discovery tool for application in the drug discovery workflow.

In some variations, the subsequent drug discovery tool is determined based on one or more of an initial input or subsequent inputs received from the end user invoking the drug discovery workflow.

In some variations, the artificial intelligence (AI) agent continues orchestrating the drug discovery workflow by at least determining to reuse an output from one or more previous applications of the subsequent drug discovery tool.

In some variations, the subsequent drug discovery tool is determined based at least on one or more benchmarks indicative of a performance of the subsequent drug discovery tool.

In some variations, the one or more benchmarks are determined based at least in part on passive feedback and/or active feedback associated with the subsequent drug discovery tool.

In some variations, the prompt is generated to display one or more preconfigured inputs for selection by the end user.

In some variations, the artificial intelligence (AI) agent comprises a primary artificial intelligence (AI) agent that orchestrates the drug discovery workflow by at least determining a sequence in which to apply the one or more drug discovery tools.

In some variations, the artificial intelligence (AI) agent orchestrates the drug discovery workflow by at least routing, based at least on the sequence in which to apply the one or more drug discovery tools, an agent flow to one or more specialized artificial intelligence (AI) agents, and wherein each specialized artificial intelligence (AI) agent orchestrates an application of a corresponding drug discovery tool.

In some variations, a specialized artificial intelligence (AI) agent routes the agent flow back to the primary artificial intelligence (AI) agent upon determining that a query from an end user exceeds a scope of the corresponding drug discovery tool.

In some variations, the executing the artificial intelligence (AI) agent includes adding, to one or more conversation turns with the artificial intelligence (AI) agent, one or more nodes guiding the artificial intelligence (AI) agent to verify a critical action, follow a sequence of the drug discovery workflow, and/or handle hallucinated values generated by one or more language models.

In some variations, a quantity of new data acquired since a previous update of a drug discovery tool is determined to satisfy one or more thresholds. The drug discovery tool is updated by at least retraining a computation model comprising the drug discovery tool.

In some variations, the drug discovery tool is incrementally updated by at least retraining the computation model based on the new data acquired since the previous update of the drug discovery tool.

In some variations, the drug discovery tool undergoes non-parametric optimization in which the computation model is retrained based on an entire collection of available data including the new data acquired since the previous update of the drug discovery tool.

In some variations, the artificial intelligence (AI) agent is coupled with a language model to support natural language based interactions with an end user invoking the drug discovery workflow.

In some variations, the one or more user inputs configuring the artificial intelligence (AI) agent includes configuration-level inputs or code-based inputs.

In some variations, the artificial intelligence (AI) agent is exposed, by one or more agent servers hosting the artificial intelligence (AI) agent, via streamable Hypertext Transfer Protocol (HTTP) or an application programming interface (API).

In some variations, a user interface (UI) displaying a selection of artificial intelligence (AI) agent templates is generated. One or more user inputs selecting, from the selection of artificial intelligence (AI) agent templates, an artificial intelligence (AI) agent template are received. The one or more inputs configuring the artificial intelligence (AI) agent are received via one or more editable fields in the artificial intelligence (AI) agent template.

In some variations, a user interface (UI) displaying the selection of artificial intelligence (AI) agents is generated. The one or more user inputs selecting the artificial intelligence (AI) agent is received via the user interface (UI).

In sone variations, the artificial intelligence (AI) agent orchestrates the drug discovery workflow by at least registering, with a molecule registration system, a molecule design generated by applying a drug discovery tool in the drug discovery workflow, and applying a subsequent drug discovery tool in the drug discovery workflow by at least providing, as input to the subsequent drug discovery tool, a unique molecule identifier assigned to the molecule design by registering with the molecule registration system.

In some variations, the subsequent drug discovery tool retrieves, from the molecule registration system, data associated with the molecule design based at least on the unique molecule identifier of the molecule design, and wherein the subsequent drug discovery tool determines an output based at least on the data retrieved from the molecule registration system.

In some variations, the unique molecule identifier includes a system identifier and a semantic identifier.

In some variations, the unique molecule identifier includes a concept identifier of a concept of the molecule design.

In some variations, the concept of the molecule design corresponds to a conceptualized version of a corresponding molecule.

In some variations, the concept of the molecule design is represented by one or more text-based notations comprising a Simplified Molecular Input Line Entry System (SMILES) string or an International Chemical Identifier (InChl) where the molecule design is a small molecule drug compound or a hierarchical editing language for macromolecules (HELM) string or an amino acid residue sequence where the molecule design is a protein molecule.

In some variations, data associated with the molecule design is linked to data associated with one or more synthesized batches of the molecule design based at least on the concept identifier of the molecule design.

In some variations, the data associated with the molecule design is stored separately as data associated with a plurality of individual components of the molecule design.

In some variations, the concept identifier of the molecule design further links the data associated with each component of the plurality of components of the molecule design.

In some variations, the plurality of components of the molecule design includes at least one of a core structure of a small molecule drug compound, one or more substructures of the core structure, and a salt version of the small molecule drug compound.

In some variations, the plurality of components of the molecule design includes one or more of an antigen binding fragment (Fab), a crystallizable fragment (Fc), a heavy chain, a light chain, a framework region, a variable region, and a complementarity determining regions (CDR).

In some variations, the registering the molecule design with the molecule registration system includes storing, in a repository associated with the molecule registration system, data associated with the molecule design.

In some variations, the repository comprises a relational database. The data associated with the molecule design is stored across a plurality of database tables. A record associated with the molecule design in one database table is linked to a record associated with the molecule design in one or more other database tables using a foreign key comprising a concept identifier of the molecule design.

In some variations, the plurality of database tables includes one or more of a table storing a concept of the molecule design, a table storing one or more synthesized batches of the molecule design, and a table storing each component of the molecule design.

In some variations, the one or more drug discovery tools include a property computation model trained to determine, based at least on a contextual information, one or more properties of interest present in a molecule design.

In some variations, the property computation model comprises a language model trained to operate on a natural language query including the contextual information.

In some variations, the contextual information includes a description of one or more controlled measurement parameters of an experimental environment in which the one or more properties of interest are measured.

In some variations, the one or more controller measurement parameters include temperature, equipment specifications, equipment settings, experimental protocols, experimental specifications, experiment-related scientific notes, and/or experimental observations.

In some variations, the property computation model comprises a reasoning model trained to decompose, into a sequence of operations, a task comprising the determining of the one or more properties of interest present in the molecule design.

In some variations, the property computation model is trained to reduce an objective function including one or more outcome terms to reduce a discrepancy between a value of the one or more properties of interest output by the property computation model and one or more ground-truth values.

In some variations, the property computation model is trained to reduce an objective function including one or more process terms to reduce a discrepancy between a sequence of operations performed by the property computation model to determine the one or more properties of interest and a corresponding ground-truth sequences of operations.

In some variations, the property computation model is further trained to determine the one or more properties of interest present in a plurality of modalities of molecule designs.

The plurality of modalities include small molecule drug compounds, therapeutic proteins or peptides, nucleic acids, multispecific antibodies, and/or bioconjugates.

In some variations, the property computation model comprises a multi-property computation trained to perform a plurality of tasks. Each task of the plurality of tasks corresponds to determining a property of interest of a plurality of properties of interest.

In some variations, the artificial intelligence (AI) agent orchestrates the drug discovery workflow by at least encoding one or more molecules designs to generate one or more corresponding embeddings for ingestion by the one or more drug discovery tools included in the drug discovery workflow.

In some variations, the artificial intelligence (AI) agent applies a universal molecular encoder to encode the one or more molecule designs. The universal molecular encoder is trained to encode multiple modalities of molecule designs.

In some variations, the artificial intelligence (AI) agent and the drug discovery workflow are deployed for executing at one or more computing nodes.

In some variations, the deploying the artificial intelligence (AI) agent and the drug discovery workflow includes packaging, into one or more software containers, each operation executing the artificial intelligence (AI) agent or a computation model of the one or more computation models in the drug discovery workflow.

In some variations, the packaging includes generating, for inclusion in a software container, a configuration file specifying a base image, one or more dependencies, and an environment setup for a corresponding operation.

In some variations, the drug discovery workflow is defined as a directed acyclic graph (DAG). Each node in the directed acyclic graph corresponds to an operation encapsulated in a software container.

In some variations, computational resources are allocated for executing, at the one or more computing nodes, the artificial intelligence (AI) agent to orchestrate the drug discovery workflow.

In some variations, the allocating the computational resources includes vertical scaling to increase computation power of one or more existing computing nodes.

In some variations, the computation power of an existing computing node is increased by at least adding more processing units and/or memory.

In some variations, the allocating the one or more computational resources includes horizontal scaling by at least adding one or more additional computing nodes.

In some variations, the artificial intelligence (AI) agent and the drug discovery workflow are associated with one or more of a training pipeline, real time inference pipeline, and batch inference pipeline. A job executing a pipeline is packaged in a software container for deployment to the one or more computing nodes for execution.

In some variations, the drug discovery workflow includes applying a molecule design computation model to generate one or more molecule designs from an input molecule, applying one or more property computation models to determine one or more properties of interest present in each molecule design generated by the molecule design computation model, and a selection computation model that selects a subset of molecule designs for experimental synthesis and testing based on the one or more properties of interest.

In some variations, the drug discovery workflow further includes a structure computation model trained to predict a structure of each molecule design in complex with one or more other molecules.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the design of drug molecules, including small molecule drug compounds and therapeutic proteins, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a drug discovery system, in accordance with some example embodiments; and

FIG. 2A depicts a schematic diagram illustrating an example of a controller interfacing with different users of a drug discovery ecosystem, in accordance with some example embodiments;

FIG. 2B depicts a schematic diagram illustrating an example of a controller interfacing with different users of a drug discovery ecosystem, in accordance with some example embodiments;

FIG. 2C depicts a schematic diagram illustrating an example of an artificial intelligence agent orchestrating a drug discovery workflow, in accordance with some example embodiments;

FIG. 3A depicts a schematic diagram illustrating examples of tasks associated with builders, in accordance with some example embodiments;

FIG. 3B depicts a schematic diagram illustrating examples of tasks associated with end users, in accordance with some example embodiments;

FIG. 4A depicts a screenshot of an example of a user interface (UI) for a drug discovery tool marketplace, in accordance with some example embodiments;

FIG. 4B depicts a screenshot of an example of a user interface (UI) for an artificial intelligence (AI) agent marketplace, in accordance with some example embodiments;

FIG. 5A depicts a flowchart illustrating an example of a process for an artificial intelligence (AI) agent orchestrated drug discovery workflow, in accordance with some example embodiments;

FIG. 5B depicts a flowchart illustrating an example of a process for an artificial intelligence (AI) agent orchestrated drug discovery workflow, in accordance with some example embodiments;

FIG. 6A depicts a flowchart illustrating an example of a process for intention-aware determination of action sequences in a drug discovery workflow, in accordance with some example embodiments;

FIG. 6B depicts a flowchart illustrating an example of a process for universal molecule registration, in accordance with some example embodiments;

FIG. 7A depicts a schematic diagram illustrating the system design of an example of a drug discovery ecosystem, in accordance with some example embodiments;

FIG. 7B depicts a schematic diagram illustrating an example of an artificial intelligence (AI) agent design that utilizes a synthesized intention-action pool to inform an artificial intelligence (AI) agent of possible trajectories mapping high-level intensions with concrete operations in a drug discovery workflow, in accordance with some example embodiments;

FIG. 8 depicts a schematic diagram illustrating an example of a drug discovery workflow orchestrated by an artificial intelligence (AI) agent, in accordance with some example embodiments;

FIG. 9A depicts a screenshot of an example of a user interface (UI) for interacting with an artificial intelligence (AI) agent for orchestrating a drug discovery workflow, in accordance with some example embodiments;

FIG. 9B depicts a screenshot of another example of a user interface (UI) for interacting with an artificial intelligence (AI) agent for orchestrating a drug discovery workflow, in accordance with some example embodiments;

FIG. 9C depicts a screenshot of another example of a user interface (UI) for interacting with an artificial intelligence (AI) agent for orchestrating a drug discovery workflow, in accordance with some example embodiments;

FIG. 9D depicts a screenshot of another example of a user interface (UI) for interacting with an artificial intelligence (AI) agent for orchestrating a drug discovery workflow, in accordance with some example embodiments;

FIG. 9E depicts a screenshot of another example of a user interface (UI) for interacting with an artificial intelligence (AI) agent for orchestrating a drug discovery workflow, in accordance with some example embodiments;

FIG. 10A depicts a screenshot of an example of a user interface (UI) for interacting with a drug discovery tool, in accordance with some example embodiments;

FIG. 10B depicts a screenshot of an example of a user interface (UI) for configuring a drug discovery tool, in accordance with some example embodiments;

FIG. 11A depicts a screenshot of an example of a user interface (UI) for configuring an artificial intelligence (AI) agent for orchestrating a drug discovery workflow, in accordance with some example embodiments;

FIG. 11B depicts a screenshot of another example of a user interface (UI) for configuring an artificial intelligence (AI) agent for orchestrating a drug discovery workflow, in accordance with some example embodiments;

FIG. 11C depicts a screenshot of another example of a user interface (UI) for configuring an artificial intelligence (AI) agent for orchestrating a drug discovery workflow, in accordance with some example embodiments;

FIG. 11D depicts a screenshot of another example of a user interface (UI) for configuring an artificial intelligence (AI) agent for orchestrating a drug discovery workflow, in accordance with some example embodiments;

FIG. 11E depicts a screenshot of another example of a user interface (UI) for configuring an artificial intelligence (AI) agent for orchestrating a drug discovery workflow, in accordance with some example embodiments;

FIG. 11F depicts a screenshot of another example of a user interface (UI) for configuring an artificial intelligence (AI) agent for orchestrating a drug discovery workflow, in accordance with some example embodiments;

FIG. 11G depicts a screenshot of another example of a user interface (UI) for configuring an artificial intelligence (AI) agent for orchestrating a drug discovery workflow, in accordance with some example embodiments;

FIG. 11H depicts a screenshot of another example of a user interface (UI) for configuring an artificial intelligence (AI) agent for orchestrating a drug discovery workflow, in accordance with some example embodiments;

FIG. 12A depicts a schematic diagram illustrating the system design of an example of a drug discovery ecosystem, in accordance with some example embodiments;

FIG. 12B depicts a schematic diagram illustrating the architecture of an example of a deployment platform in a drug discovery ecosystem, in accordance with some example embodiments; and

FIG. 13 depicts a block diagram illustrating an example of a computing system, in accordance with some example embodiments.

When practical, similar reference numbers denote similar structures, features, or elements.

### DETAILED DESCRIPTION

The primary goal of drug discovery includes the creation of safe and effective drug molecules capable of binding to and modulating (e.g., activating, inhibiting, and/or the like) one or more biological targets including, for example, receptors, enzymes, ion channels, nucleic acids, and/or the like. For example, in some cases, a drug molecule may be designed to exhibit one or more drug-like properties including, for example, binding affinity, specificity, biological activity, developability, and/or the like. Conventional drug design typically resorts to evaluating molecule designs from a solution space of possible molecule designs. However, the solution space for possible molecule designs, while almost infinitely large and scales exponentially with molecule size, is also sparsely populated by those with drug-like properties.

For small molecule drugs, for example, the molecular space (or chemical space) occupied by every possible chemical compound (e.g., every possible combination of atoms of two or more chemical elements) is estimated to contain 10⁶⁰ possible chemical compounds, meaning that state-of-the-art computational resources can support the exploration of only a small fraction of the molecular space (e.g., small regions of the molecular space selected based on prior domain knowledge). The size of the combinatorial space is magnitudes larger for larger molecules, such as peptides and proteins. For example, for protein molecules containing an N quantity of amino acid residues, approximately 20^{N} possible protein sequences exist when each of the N quantity of amino acid residues is one of the twenty canonical amino acid residues. Each one of the aforementioned 20^{N} possible protein sequences is further capable of adopting an exponential number of conformations (or three-dimensional structures). Even in cases where each one of the N quantity of amino acid residues in a possible protein sequence is limited to assuming one of an M quantity of discrete geometric states (e.g., rotamers), every one of the aforementioned 20^{N} possible protein sequences may still adopt M^{N} possible conformations (or three-dimensional structures). Given the vast scale of the solution space, even state-of-the-art computational resources can only support the exploration of a small fraction of the solution space. Such limitations in search scope means that conventional search-based approaches are not optimized for identifying molecule designs with the most optimal drug-like properties.

In light of the aforementioned challenges, modern drug discovery efforts may leverage a variety of drug discovery tools to accelerate tasks such as computational models for molecule design generation and modification, molecular property prediction, candidate molecule ranking and selection, and/or the like. However, the diversity and fragmentation of drug discovery tools may hinder the goals of drug discovery. For example, drug discovery tools developed by independent developers may be trained on different datasets and implemented using diverse architectures. Moreover, different drug discovery tools may also exhibit varying degrees of domain specificity. This fragmentation erects barriers to tool accessibility and usability, which in turn disrupt drug discovery workflows to the point that tools can only be operated individually and often in isolation. Amongst the many challenges to integrating an ever increasing number of available tools is that drug discovery in practice tends to be an open-ended exploration that requires the dynamic selection and orchestration of suitable drug discovery tools.

Various example embodiments of the present disclosure include a drug discovery ecosystem with infrastructure to support the design and deployment of artificial intelligence (AI) agents for dynamic selection and orchestration of drug discovery tools. In some cases, various example embodiments of the drug discovery ecosystem disclosed herein may support the design and deployment of an artificial intelligence (AI) agent interfacing with a large-scale and evolving drug discovery tool collection with scientific intention awareness. Whereas the fragmentation of current state-of-the-art drug discovery tools renders molecule design feedback loops impossible, consolidating diverse drug discovery tools in a single drug discovery ecosystem in which suitable drug discovery tools are deployed in automated, intention-aware drug discovery workflows better supports the feedback loops for incrementally refining molecule designs over successive design iterations. In some cases, the drug discovery tool collection may include computation models for molecule design generation and modification, molecular property prediction, candidate molecule ranking and selection, and/or the like. In some cases, the drug discovery tool collection may cover the spectrum of molecule design operations, including both large molecules (e.g., therapeutic proteins) and small molecule drug compounds. In some cases, the drug discovery tools in the drug discovery tool collection may, individually or in combination, address a multitude of drug discovery objectives including, for example, hit expansion, lead optimization, compound filtering and ranking, and/or the like. As described in more detail below, in some cases, the artificial intelligence (AI) agent may be designed to navigate a drug discovery action space defined by the capabilities supported by the available drug discovery tools in the drug discovery tool collection. For instance, in some cases, artificial intelligence (AI) agent may leverage one or more language models (e.g., large language models (LLMs)) to orchestrate, based on one or more user inputs, a complex sequence of actions utilizing one or more drug discovery tools from the drug discovery tool collection.

In some example embodiments, the drug discovery ecosystem may include a controller interfacing with one or more users interacting with the drug discovery ecosystem, including with one or more of the artificial intelligence (AI) agents and drug discovery tools (e.g., in the drug discovery tool collection) accessible through the drug discovery ecosystem. In some cases, the controller may interface with a variety of different users (and types of users), including those with different roles, skillsets, objectives, and/or the like. Examples of users (or types of users) include developers, builders, end users, and/or the like. For example, in some cases, the controller may receive, from a builder, one or more inputs configuring a new drug discovery tool, a new capability to an existing drug discovery tool, a new tool server (e.g., a Model Context Protocol (MCP) server), a new drug discovery workflow, an artificial intelligence (AI) agent, and/or the like. Furthermore, in some cases, the controller may implement, based on the configurations specified in the one or more inputs from the builder, the new drug discovery tool, the new tool server, the new capability to the existing drug discovery tool, the new drug discovery workflow, the artificial intelligence (AI) agent, and/or the like. In some cases, the controller may deploy, for consumption by one or more end users of the drug discovery ecosystem, the new drug discovery tool, the new tool server, the new capability to the existing drug discovery tool, the new drug discovery workflow, the artificial intelligence (AI) agent, and/or the like.

In some example embodiments, the controller may provide, to one or more end users, access to one or more artificial intelligence (AI) agents, drug discovery tools, drug discovery workflows, and/or the like. For example, in some cases, the controller may bind an artificial intelligence (AI) agent (e.g., configured by a builder) to one or more language models (e.g., large language models (LLMs)) such that an end user may interact with the artificial intelligence (AI) agent using natural language queries. For example, in some cases, the artificial intelligence (AI) agent may respond to a natural language query from the end user by at least determining a corresponding drug discovery workflow (e.g., a predetermined drug discovery workflow, an adaptive drug discovery workflow, and/or the like) to achieve one or more objectives of the natural language query. In some cases, the drug discovery workflow may invoke one or more drug discovery tools to perform a sequence of actions for achieving the one or more objectives of the natural language query. Accordingly, in some cases, the artificial intelligence (AI) agent may orchestrate the drug discovery workflow by at least interfacing with one or more drug discovery tools implementing the drug discovery workflow.

In some example embodiments, the controller may allocate computational resources, such as graphics processing unit (GPU) access, to support the drug discovery workflow orchestrated by the artificial intelligence (AI) agent. For example, in some cases, the controller may classify the drug discovery workflow as one or more of a training (or retraining) workflow, a real-time (or online) inference workflow, or a batch (offline) inference workflow. In some cases, each of the foregoing workflow may be associated with different computational resource requirements and traffic patterns. Accordingly, in some cases, the controller may allocate the necessary computational resources through horizontal scaling (e.g., adding more computing machines or nodes) and/or vertical scaling (e.g., increasing power of existing computing machines, such as adding more processing units, memory, storage, and/or the like). In some cases, the drug discovery workflow may be defined as directed acyclic graphs (DAGs), with each operation in the drug discovery workflow containerized, for example, in one or more application containers (e.g., Docker containers). In some cases, an application container may be a standardized, isolated, and self-contained runtime environment that packages an application (e.g., a drug discovery tool in the drug discovery workflow) and all its dependencies, thereby enabling the application to be run consistently in any computing environment. In some cases, one or more application containers (e.g., Docker containers) may be grouped into logical units (e.g., Pods). In some cases, the controller may deploy the application containers (e.g., organized into Pods) into a computing environment (e.g., clusters of computing nodes), schedule the running of application containers, scale computational resources (e.g., horizontal and vertical scaling), perform self-healing (e.g., restart failed application containers), and load balance (e.g., across computing nodes or clusters of computing nodes).

In some example embodiments, the artificial intelligence (AI) agent may orchestrate a drug discovery workflow in which multiple drug discovery tools operate on a molecule design. For example, in some cases, the drug discovery workflow may include applying a molecule design computation model to generate a molecule design before a property computation model is applied to predict one or more properties of the molecule design. In some cases, the drug discovery ecosystem may include a therapeutic molecule registration (TMR) system providing centralized and uniform access to data associated with the molecule design including, for example, chemical formulation, amino acid sequence, three-dimensional structure and/or substructure, properties, lineage, and/or the like. Accordingly, instead of transferring the data associated with the molecule design between the molecule design computation model and the property computation model, which consumes bandwidth and increases latency, this data may be accessible to each computation model from a central repository (e.g., a relational database) based on a unique, persistent molecule identifier. For instance, in some cases, the data associated with the molecule design generated by the molecule design computation model may be uploaded to the therapeutic molecule registration (TMR) system and stored in the central repository (e.g., the relational database) along with a corresponding molecule identifier. Moreover, in some cases, the property computation model may access at least a portion of the data associated with the molecule design using the molecular identifier associated with the molecule design.

In some example embodiments, the therapeutic molecule registration (TMR) system may store molecule designs in accordance with a hierarchical data model that is agnostic to the modality of the molecule designs. For example, in some cases, the hierarchical data model may support the storage of small molecule drug compounds, large molecules (e.g., therapeutic peptides and proteins), multispecific antibodies, bioconjugates (e.g., antibody drug conjugates (ADCs)), and/or the like. In some cases, a modality agnostic data model may benefit from cross-modality insights such as, for example, patterns that may be reused across the storage of different molecular modalities, drug discovery workflows operating on different molecular modalities, and/or the like. In some cases, each molecule design stored in the therapeutic molecule registration (TMR) system may be assigned a unique system identifier and a corresponding semantic identifier, the latter having semantic significance to end users. In some cases, in accordance with the hierarchical data model, the system identifier and the semantic identifier of a molecule design may include a concept identifier of a concept corresponding to a conceptualized version of the corresponding molecule. In some cases, the conceptualized version of a molecule design may be represented by one or more text-based notations such as a Simplified Molecular Input Line Entry System (SMILES) string or an International Chemical Identifier (InChl) for a small molecule drug compound or an amino acid residue sequence or a hierarchical editing language for macromolecules (HELM) string for a protein molecule. In some cases, where the molecule design is synthesized, each batch of the synthesized molecule design may be assigned a unique system identifier and semantic identifier having a corresponding batch identifier. In some cases, the therapeutic molecule registration (TMR) system may store a single molecule design as multiple constituent components. For instance, in some cases, a small molecule drug compound may be stored as a core structure (e.g., the active drug molecule) and the corresponding salt form (e.g., formed by reacting the core structure with an acid or base to yield a neutral, crystalline structure with improved properties for drug delivery), each of which being associated with a unique system identifier and semantic identifier. Alternatively, a protein molecule, such as an antibody, may be stored as a heavy chain and a light chain, each of which being associated with unique system identifier and semantic identifier. As described in more detail below, the central repository associated with the therapeutic molecule registration (TMR) system may include a relational database in which data associated with a molecule design, including the constituent components of the molecule and one or more batches of the synthesized molecule design, are stored across two or more tables linked by keys (e.g., primary keys, foreign keys, and/or the like).

In some example embodiments, the drug discovery tool collection may include a multi-property computation model trained to determine multiple properties of a molecule design including, for example, drug-like properties such as binding affinity, specificity, biological activity, developability, and/or the like. For example, in some cases, the multi-property computation model may be trained to perform multiple tasks, each of which corresponding to the prediction of a property of interest. Furthermore, in some cases, the multi-property computation model may also be multi-modal, meaning that it is capable of determining the properties of interest for multiple molecular modalities (e.g., small molecule drug compounds, therapeutic peptides and proteins, nucleic acids, and/or the like). In some cases, the drug discovery tool collection may include the multi-property computation model instead of multiple individual property computation models trained to predict single properties of interest or multiple single modality property computation models. In some cases, the multi-property computation model may perform better than individual property computation models and single modality property computation models at least because the multi-property computation model may benefit from transfer learning across different tasks.

In some example embodiments, the multi-property computation model may be trained to determine one or more properties of interest based on the molecular representation of a molecule design. Furthermore, in addition to the molecular representation of the molecule design, the multi-property computation model may be trained to determine the one or more properties of interest based on contextual information. Examples of contextual information may include descriptions of one or more controlled measurement parameters of the experimental environment in which the properties of interest are measured, such as temperature, equipment specifications, equipment settings, experimental protocols, experimental specifications, experiment-related scientific notes, experimental observations, and/or the like. In some cases, contextual information may be better described in natural language. Accordingly, in some cases, various example embodiments of the multi-property computation model described herein may be implemented using a language model (e.g., a large language model) trained to operate on natural language inputs. That the multi-property computation model determines the one or more properties of interest based on contextual information is unlike conventional property computation models whose predictions disregard the experimental environment in which the one or more properties of interest are measured despite the confounding factors present in these experimental environments.

In some example embodiments, the multi-property computation model may be a reasoning model (e.g., a reasoning language model (RLM)) trained to decompose the complex task of determining multiple properties of interest into multiple constituent operations. In some cases, the multi-property computation model may explore different sequences of operations for determining the properties of interest. In some cases, the multi-property computation model may be trained to optimize (e.g., reduce or minimize) an objective function with one or more outcome terms (e.g., outcome rewards) that aim to reduce (or minimize) the discrepancy between the properties of interest determined by the multi-property computation model and the corresponding ground-truth values. Alternatively and/or additionally, in some cases, the multi-property computation model may be trained to optimize (e.g., reduce or minimize) an objective function including one or more process terms (e.g., a process rewards) that aim to reduce (or minimize) the discrepancy between the sequences of operations performed by the multi-property computation model to determine the properties of interest and the corresponding ground-truth sequences of operations.

In some example embodiments, the drug discovery tool collection may include tools, such as computation models, that operates on embeddings of molecule designs rather than directly on conventional molecular representations. In some cases, expressive computation models capable of capturing the complexity of the patterns and variations across molecular structures tend to be large scale models with numerous parameters (e.g., weights, biases, and/or the like). For example, some conventional computation models may be trained to operate on structural representations of molecule designs that includes the spatial coordinates (e.g., Cartesian coordinates (x,y,z)) of each constituent atom. However, computation models capable of operating on such atomic resolution structural representations necessitate large, bespoke model architectures that impose significant computational burden. Large computation models also require massive quantities of training data to achieve satisfactory performance. To reduce the computation burden and improve the performance of computation models operating in low data regimes the quantity of available training data is limited, at least some of the computation models included in the drug discovery tool collection may operate on embeddings of molecule designs. In some cases, these computation models may operate on embeddings of molecule designs generated by a universal molecular encoder (UME) trained to generate embeddings of multiple modalities of molecules including, for example, small molecule drug compounds, therapeutic peptides and proteins, nucleic acids, and complexes thereof. As described in more detail below, in some cases, the universal molecular encoder (UME) may leverage training data across modalities to achieve improved encoding performance over single modality encoders. For instance, in some cases, the universal molecular encoder (UME) may generate embeddings that better capture critical molecular features than conventional single modality encoders.

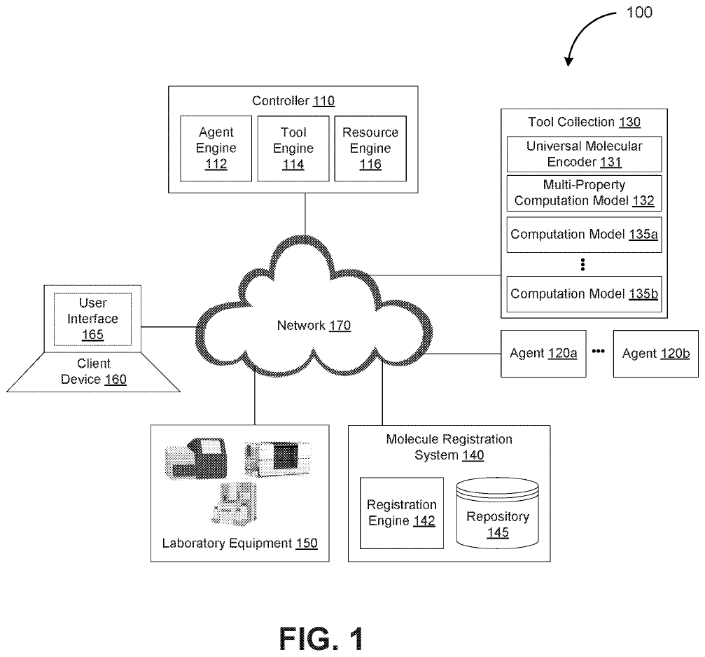

FIG. 1 depicts a system diagram illustrating an example of a drug discovery ecosystem 100, in accordance with some example embodiments. Referring to FIG. 1, the drug discovery ecosystem 110 may include a controller 110, one or more agents 120, a tool collection 130, a molecule registration system 140, one or more laboratory equipment 150, and a client device 160. As shown in FIG. 1A, the controller 110, the one or more agents 120, the tool collection 130, the molecule registration system 140, the one or more laboratory equipment 150, and the client device 160 may be communicatively coupled via a network 170.

In some cases, the one or more laboratory equipment 150 may include any wetlab and dry lab equipment capable of synthesis, purification, and/or analysis. Examples of the one or more laboratory equipment 150 may include synthesizers, including standard equipment (e.g., fume hoods, glassware, heating and cooling devices, stirrers), automated and specialized synthesis platforms (e.g., automated synthesizers, parallel synthesis workstations, and high-throughput experimentation (HTE) for efficient reaction optimization, and specialized reactors (e.g., microwave, flow, photochemistry). In some cases, the one or more laboratory equipment 130 may also include tools to support various purification techniques such as chromatography (e.g., silica gel chromatography, preparative high performance liquid chromatography (Prep HPLC)), centrifugal, crystallization/recrystallization, and/or the like. In some cases, the one or more laboratory equipment 150 may also include analytical tools such as microscopes, spectroscopes, spectrometers (e.g., nuclear magnetic resonance (NMR) spectrometers, mass spectrometers), balances, pH meters, elemental analyzers, and/or the like.

In some cases, the client device 160 may be a processor-based device including, for example, a workstation, a desktop computer, a laptop computer, a smartphone, a tablet computer, a wearable apparatus, and/or the like. In some cases, the network 170 may be a wired network and/or a wireless network including, for example, a local area network (LAN), a virtual local area network (VLAN), a wide area network (WAN), a public land mobile network (PLMN), the Internet, and/or the like.

In some example embodiments, the controller 110 may interface with one or more users interacting with the drug discovery ecosystem 100 via, for example, the client device 160. In some cases, the controller 110 may interface with a variety of different users (and types of users), including those with different roles, skillsets, objectives, and/or the like. Examples of users (or types of users) include developers, tool builders, end users, and/or the like. For example, in some cases, the controller 110 may receive one or more inputs provided by a builder via the user interface 165 at the client device 160. In some cases, the one or more inputs may configure a new drug discovery tool, a new capability to an existing drug discovery tool, a new tool server (e.g., a Model Context Protocol (MCP) server), a new drug discovery workflow, an artificial intelligence (AI) agent orchestrating a drug discovery workflow, and/or the like. Furthermore, in some cases, the controller 110 may implement, based on the configurations specified in the one or more inputs from the builder, the new drug discovery tool, the new tool server, the new capability to the existing drug discovery tool, the new drug discovery workflow, the artificial intelligence (AI) agent, and/or the like. In some cases, the controller 110 may deploy, for consumption by one or more end users of the drug discovery ecosystem 100, the new drug discovery tool, the new tool server, the new capability to the existing drug discovery tool, the new drug discovery workflow, the artificial intelligence (AI) agent, and/or the like.

Referring again to FIG. 1, in some example embodiments, the controller 110 may include an agent engine 112 that implements, for example, one or more agents 120, based on the one or more user inputs received from the builder via the user interface 165 at the client device 160. In some cases, the controller 110 may further include a tool engine 114 that implements one or more tools, such as one or more computation models 135 in the tool collection 130, based on the one or more user inputs received from the builder via the user interface 165. As shown in FIG. 1, in some cases, the controller 110 may further include a resource engine 116 that allocates, scales, and maintains computational resources (e.g., processing units, memory, storage, and/or the like) to support the operations of the agent 120 and the computation models 135 selected from the tool collection 130 to implement one or more drug discovery workflows.

In some example embodiments, the tool collection 130 may include multiple computation models including, for example, a first computation model 135a, a second computation model 135b, and/or the like. In some cases, the first computation model 135a and the second computation model 135b may be trained to perform one or more drug discovery tasks. For example, in some cases, the first computation model 135a may be a generative model trained to generate (or modify) one or more molecule designs (e.g., small molecule drug compounds, therapeutic proteins and peptides, nucleic acids, and/or the like). Furthermore, in some cases, the second computation model 135b may be a classifier or a predictive model trained to determine one or more properties of interest present, for example, in the molecule designs generated by the first computation model 135a.

In some example embodiments, the tool collection 130 may include one or more multimodal computation models capable of operating on (e.g., generate, modify, predict the properties of, and/or the like) multiple molecular modalities including, for example, small molecule drug compounds, therapeutic proteins and peptides, nucleic acids, and/or the like. For instance, as shown in FIG. 1, the tool collection 130 may include a universal molecular encoder 131 trained to generate embeddings of multiple modalities of molecules for use by one or more other computation models included in the tool collection 130. In some cases, the tool collection 130 may also include a multi-property computation model 132 trained to trained to determine multiple properties of a molecule design including, for example, drug-like properties such as binding affinity, specificity, biological activity, developability, and/or the like. In some cases, the multi-property computation model 132 may be capable of operating on multiple modalities of molecules. Moreover, as described in more detail below, the multi-property computation model 132 may be a reasoning model trained to increase (or maximize) an objective function having one or more of an outcome term (e.g., an outcome reward), process term (e.g., process reward), and/or the like. For example, in some cases, a drug discovery workflow may include applying a first computation model 135a (e.g., a molecule design computation model) to generate a molecule design before a second computation model 135b (e.g., a property computation model) is applied to predict one or more properties of the molecule design.

In some example embodiments, a drug discovery workflow, such as one orchestrated by the one or more agents 120, may include multiple drug discovery tools operating on a same molecule design. In some cases, the drug discovery ecosystem 100 may include the molecule registration system 140 to facilitate access to and the transfer of data associated with the molecule design across different computation models in the drug discovery workflow. For instance, in some cases, the molecule registration system 140 may provide centralized and uniform access to data associated with the molecule design including, for example, chemical formulation, amino acid sequence, three-dimensional structure and/or substructure, properties, lineage, and/or the like. Accordingly, instead of transferring the data associated with the molecule design between the first computation model 135a and the second computation model 135b, which consumes bandwidth and increases latency, data associated with the molecule design may be accessible to each computation model from a repository 145 (e.g., a relational database) based on a unique, persistent molecule identifier. For instance, in some cases, the data associated with the molecule design generated by the first computation model 135a (e.g., the molecule design computation model) may be uploaded to the molecule registration system 140 where a registration engine 142 assigns the unified, persistent molecule identifier before the molecule design and its corresponding data are stored in the repository 145 (e.g., the relational database) along with the molecule identifier. In some cases, the second computation model 135b (e.g., the property computation model) may access at least a portion of the data associated with the molecule design from the repository 145 (e.g., the relational database) using the molecular identifier associated with the molecule design.

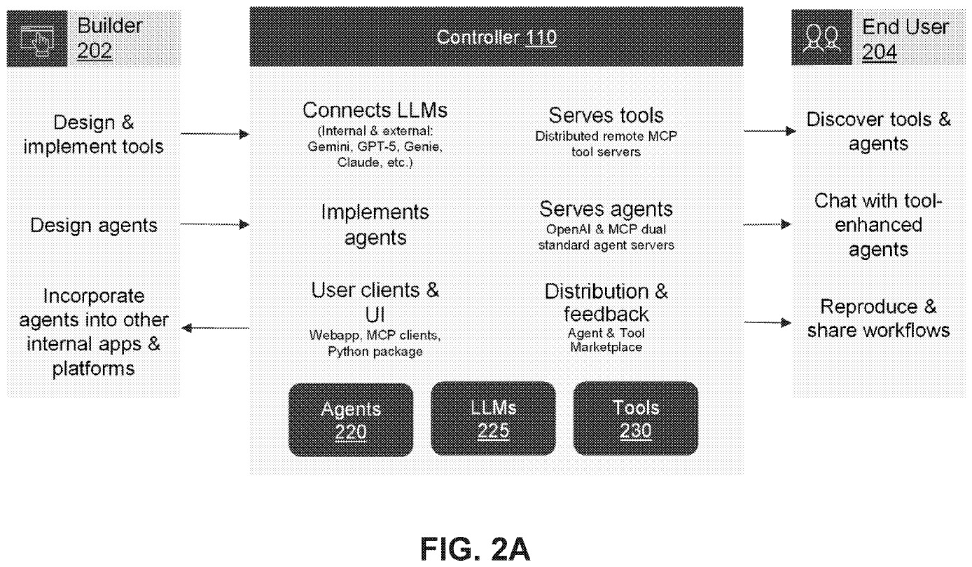

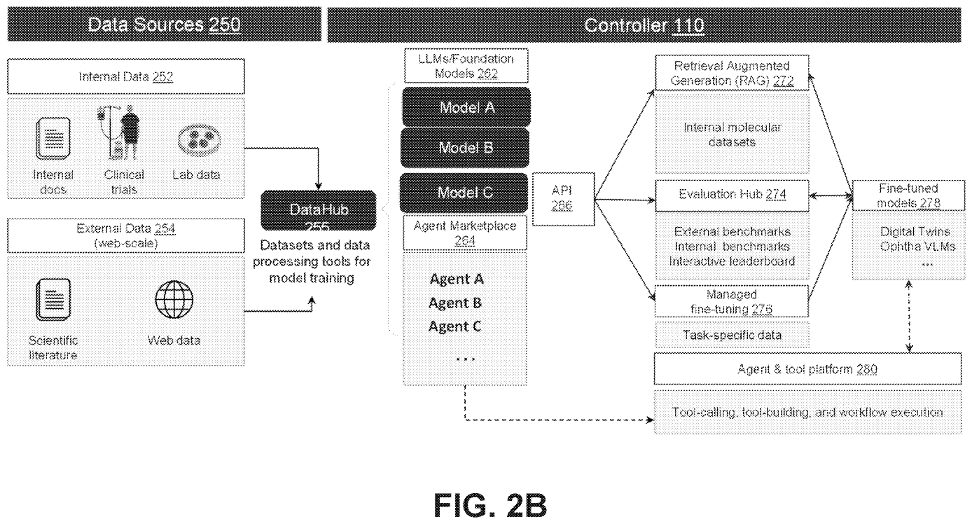

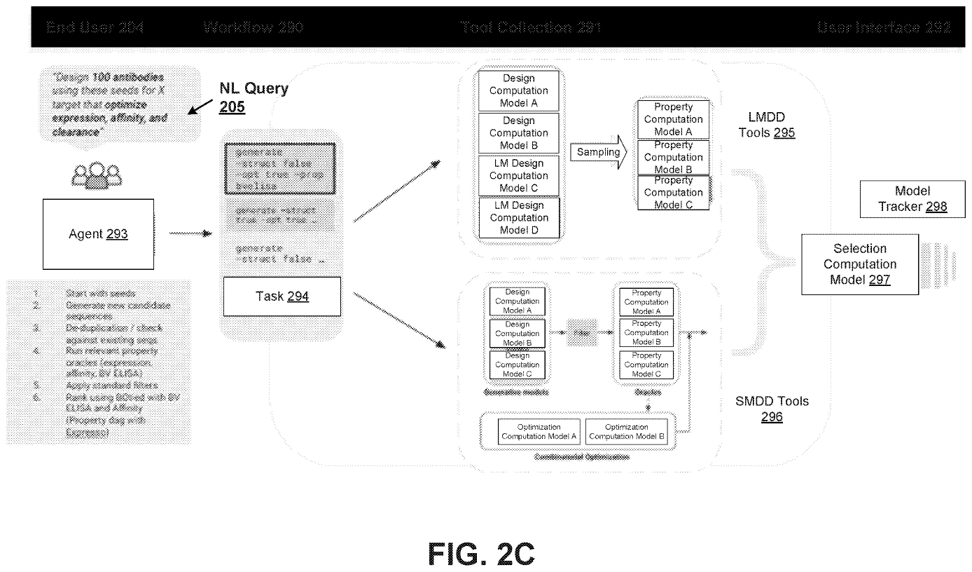

As noted, in some example embodiments, the controller 110 may interface with a variety of different users (or types of users) including those with different roles, skillsets, objectives, and/or the like. Examples of users (or types of users) include developers, builders, end users, and/or the like. To further illustrate, FIG. 2A depicts a schematic diagram illustrating an example of the controller 110 interfacing with different types of users. For example, FIG. 2A shows the controller 110 interfacing with a builder 202, a type of user who interacts with the drug discovery ecosystem 100 to accomplish objectives such as designing and implementing tools, designing agents for orchestrating drug discovery workflows, incorporating agents into other applications and platforms, and/or the like. In some cases, the builder 202 may send, via the client device 160, one or more inputs to the drug discovery ecosystem 100 designing and implementing tools 230, designing agents 220 for orchestrating drug discovery workflows invoking the tools 230, incorporating the agents 220 into other applications and platforms (e.g., large language models 225), and/or the like. In some cases, FIG. 2A shows that the controller 110 may respond to the one or more inputs from the builder 202 by implementing the agents 220 and connecting to the one or more large language models 225, both internal and external, to support natural language based interactions with the agents 220. As described in more detail below, in some cases, the agents 220 may orchestrate one or more drug discovery workflows to achieve the objectives specified in one or more natural language queries received from an end user 204 of the drug discovery ecosystem 100, e.g., “improve the affinity score of x molecule design.”

Referring again to FIG. 2A, in some cases, the controller 110 may also interface with the end user 204, a different type of user who interacts with the drug discovery ecosystem 100 to accomplish objectives such as discovering the agents 220 and the tools 230, chatting with tool-enhanced agents 220 with support from the large language models 225, reproducing and sharing drug discovery workflows orchestrated by the agents 220, and/or the like. For example, in some cases, the end user 204 may send, via the client device 160, one or more inputs to the drug discovery system 100 discovering the agents 220 and the tools 230, chatting with tool-enhanced agents 220 with support from the large language models 225, reproducing and sharing drug discovery workflows orchestrated by the agents 220, and/or the like. As shown in FIG. 2A, in some cases, the controller 110 may coordinate the interaction with the end user 204 by at least providing (or serving) the tools 230 (e.g., via distributed remote Model Context Protocol (MCP) servers), providing (or serving) the agents (e.g., via Model Context Protocol (MCP) servers, dual standard agent servers, and/or the like), collecting feedback (e.g., passive feedback, active feedback, and/or the like) from the end user 204, benchmarking the tools 230, and/or the like.

FIG. 2B depicts a schematic diagram that further illustrates another example of the controller 110 interfacing with different data sources 250, in accordance with some example embodiments. As shown in FIG. 2B, in some cases, the controller 110 may interface with the data sources 250 via a data hub 255. In the example shown in FIG. 2B, the data sources 250 may include internal data 252 such as internal documents, clinical trial data, lab data, and/or the like. In some cases, the data sources 250 may further include external data 254 such as scientific literature, web data, and/or the like.

Referring again to FIG. 2B, in some cases, the data hub 255 may host datasets as well as data processing tools for training one or more computation models available, for example, in the drug discovery ecosystem 100 (e.g., as part of the drug discovery tool collection 130). In the example shown in FIG. 2B, the one or more computation models may include language models (e.g., large language models (LLMs)) and foundation models 262 and artificial intelligence (AI) agents populating the agent marketplace 264. In some cases, the one or more computation models may be exposed to other resources within the drug discovery ecosystem 100 via an application programing interface (API) 266. For example, as shown in FIG. 2B, examples of such resource include retrieval augmented generation (RAG) 272 to enhance the operations of the large language models (LLMs)) and foundation models 262 with internal molecular datasets, an evaluation hub 274 to provide benchmarks and model ranking, and the managed finetuning 276 of the large language models (LLMs)) and foundation models 262 with task-specific training data. As shown in FIG. 2B, the resulting finetuned models 278 may be made available via the agent and tool platform 280 along with the large language models (LLMs)) and foundation models 262 and the agents in the agent marketplace 264. For instance, in some cases, the agent and tool platform 280 may provide functionalities such as tool calling, tool building, and drug discovery workflow execution.

FIG. 2C depicts a schematic diagram illustrating an example of a drug discovery workflow 290 orchestrated by an agent 293, in accordance with some example embodiments. As shown in FIG. 2C, in some cases, the end user 204 may initiate the drug discovery workflow 290 by at least sending, to the drug discovery ecosystem 100, a natural language query 205 specifying a task 294 (e.g., “Design 100 antibodies using these seeds for X target that optimize expression, affinity, and clearance.”). As shown in FIG. 2C, in some cases, the agent 293 may orchestrate the drug discovery workflow 290 by at least determining a sequence of operations for accomplishing the task 294 and identifying, within a tool collection 291, one or more corresponding tools. For example, in FIG. 2C, the tool collection 291 may include large molecule drug discovery (LMDD) tools 295 such as, for example, one or more design computation models, property computation models, and/or the like. In some cases, the tool collection 291 may also include small molecule drug discovery (SMDD) tools 296 including, for example, design computation models, property computation model, combinatorial optimization computation models, and/or the like. In some cases, one or more of the computation models in the tool collection 291 may be multimodal, including universal molecular encoders (UMEs) and multi-property computation models, which are capable of operating on (e.g., generate, modify, predict the properties of, and/or the like) multiple molecular modalities including, for example, small molecule drug compounds, therapeutic proteins and peptides, nucleic acids, and/or the like. In some cases, the drug discovery ecosystem 100 may include a model tracker 298 that tracks the performance of each drug discovery tool and, in some cases, user feedback (e.g., passive and active feedback from the end user 204). In some cases, passive feedback for a particular drug discovery tool may include usage metrics, such as usage frequency, whereas active feedback may include one or more ratings provided directly by an end user. In some cases, the agent 293 may orchestrate the drug discovery workflow 290 by at least selecting, based at least on the benchmarks maintained by the model tracker 298, one or more suitable drug discovery tools from the tool collection 291 to implement the drug discovery workflow 290. For instance, in some cases, the agent 293 may select one drug discovery tool over another drug discovery tool for the same task (e.g., one molecule design computation model or property computation model over another) where the one or more benchmarks indicate that the one drug discovery tool exhibits better performance than the other drug discovery tool.

Referring again to FIG. 2C, in some cases, the agent 293 may orchestrate the drug discovery workflow 290 by at least coordinating the operations of computation models including the scheduling of the computation models in an appropriate sequence and the transfer of input and output data therebetween, and interfacing with the end user 204 to obtain additional inputs required for continuing the drug discovery workflow 290. For instance, in the example of the drug discovery workflow 290 shown in FIG. 2C, the agent 293 may coordinate the application of a selection computation model 297 to rank the molecule designs generated by the design computation models selected from the tool collection 291 based on one or more properties of interest predicted by the property computation models such that those satisfying the criteria specified by end user 204 are returned to the end user 204 (e.g., via the user interface 292).

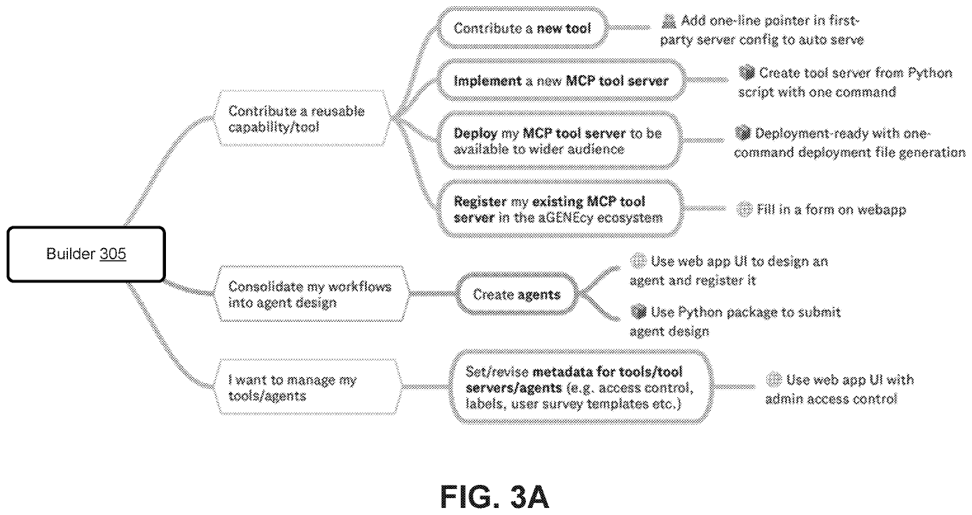

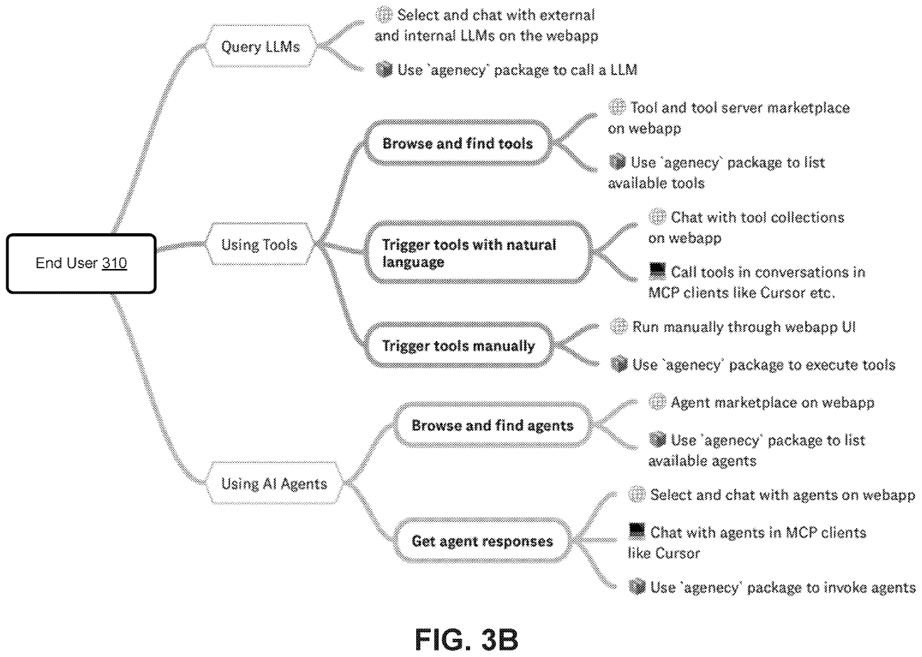

FIG. 3A depicts a schematic diagram illustrating examples of tasks associated with builders, in accordance with some example embodiments. In some example embodiments, the controller 110 may interface with a variety of different users (and types of users), including those with different roles, skillsets, objectives, and/or the like. In the example shown in FIG. 3A, the controller 110 may interface with a builder 305 to perform one or more tasks. Examples of tasks that may be performed by the builder 305 interfacing with the controller 110 may include contributing a reusable drug discovery tool, contributing a reusable capability to an existing drug discovery tool, consolidating one or more drug discovery workflows into an agent design, managing drug discovery tools and artificial intelligence (AI) agents, and/or the like.

Referring again to FIG. 3A, in some cases, a builder 305 may contribute a new drug discovery tool. As shown in FIG. 3A, in some cases, the builder 305 may configure new drug discovery tool to be served automatically, for example, to one or more end users (e.g., the end user 204 in FIG. 2A) by at least adding one or more pointers in a server configuration. In some cases, the builder 305 may also implement a new tool server (e.g., a new Model Context Protocol (MCP) server). In some cases, the controller 110 may support the creation of the new tool server from a script (e.g., Python script) with a single command from the builder 305. In some cases, the builder may further deploy the tool server such that the tool server is accessible to a wider audience of end users (e.g., end user 204 in FIG. 2A). As shown in FIG. 3A, the configurations of the new tool server may be packaged in a deployment file that is ready for deployment with, in some cases, a single command. In some cases, the builder 305 may further register the new tool server in the drug discovery ecosystem 100. In some cases, FIG. 3A shows the controller 110 may support the registration of the tool server via a fillable web-based form.

Referring again to FIG. 3A, in some cases, the builder 305 may configure one or more artificial intelligence (AI) agents to orchestrate one or more corresponding drug discovery workflows (e.g., small molecule drug discovery (SMDD) workflows, large molecule drug discovery (LMDD) workflows, and/or the like). For example, in FIG. 3A, the controller 110 may receive, from the builder 305, one or more inputs consolidating one or more drug discovery workflows into the design of an artificial intelligence (AI) agent. In some cases, the controller 110 may respond to the one or more inputs consolidating the one or more drug discovery workflows into agent design by at least creating the corresponding artificial intelligence (AI) agents. For instance, as shown in FIG. 3A, the builder 305 may design and register each artificial intelligence (AI) agent using the user interface (UI) of a web-based application. In some cases, the configurations of each agent may be submitted in a package (e.g., Python package) for implementation by the controller 110. Furthermore, FIG. 3A shows that the builder 305 may continue to use the user interface (UI) of the web-based application (e.g., with administrator access control) to manage the drug discovery tools and agents, for example, by setting and revising metadata for the drug discovery tools (e.g., tool name, description, input arguments, outputs, contributors, administrators, permissions, servers, uniform resource locator (URL), performance characteristics, and/or the like), tool servers, and artificial intelligence (AI) agents. In some cases, metadata for artificial intelligence (AI) agents may include agent identifier, agent name, description, contributors, administrators, permissions, serving type (e.g., API, code, configuration), and codebase. In some cases, the metadata for an application programming interface (API) based agent may include a corresponding uniform resource locator (URL). For a configuration level agent, the metadata may further include choice of language model, system prompt, tool choices (e.g., tool identifiers), and orchestration design. As described in more detail below, one or more of the drug discovery tools may undergo non-parametric optimization in which, instead of incremental updates that can lead to catastrophic forgetting, the one or more drug discovery tools retrained using a continuously updated dataset.

As noted, in some example embodiments, the controller 110 may interface with a variety of different users (and types of users), including those with different roles, skillsets, objectives, and/or the like. In the example shown in FIG. 3B, the controller 110 may interface with an end user 310 to perform one or more tasks. Examples of tasks that may be performed by the end user 310 interfacing with the controller 110 may include querying a language model (e.g., a large language model (LLM)), using one or more drug discovery tools, using one or more artificial intelligence (AI) agents, and/or the like. As described in more details below, at least some of the aforementioned tasks may be at least partially overlapping. For example, in some cases, the end user 310 may use a drug discovery tool directly or by querying the language model (e.g., large language model (LLM)) or by interacting with an artificial intelligence (AI) agent that orchestrate a drug discovery workflow that utilizes the drug discovery tool.

Referring again to FIG. 3B, in some cases, the end user 310 may query the language model (e.g., the large language model (LLM)) by selecting the language model, for example, from a selection of available language models (e.g., large language models (LLMs)) via a web-based application. In some cases, it may also be possible for the end user 310 to query the language model (e.g., the large language model (LLM)) through an artificial intelligence (AI) agent bound to the language model.

In some case, the end user 310 may utilize one or more drug discovery tools by first browsing the available selection of drug discovery tools (e.g., in the tool collection 130 in FIG. 1) and selecting the drug discovery tool directly for utilization. As shown in FIG. 3B, in some cases, the drug discovery ecosystem 100 may include a tool and tool server marketplace that is accessible via a web-based application. In some cases, the end user 310 may trigger the tools indirectly via the language model (e.g., the large language model (LLM)). Alternatively and/or additionally, the end user 310 may trigger the tool directly, for example, by running the tool through the user interface (UI) of the web-based application. As yet another alternative, one or more drug discovery tools may be triggered by the end user 310 interacting with an artificial intelligence (AI) agent. For example, in FIG. 3A, the end user 310 may browse and find an artificial intelligence (AI) agent in an agent marketplace accessible via the web-based application. In some cases, each artificial intelligence (AI) agent may be configured to orchestrate one or more corresponding drug discovery workflows (e.g., large molecule drug discovery (LMDD) workflow, small molecule drug discovery (SMDD) workflow, and/or the like). In some cases, the end user 310 may interact with a selected artificial intelligence (AI) agent to trigger a drug discovery workflow, which may in turn trigger one or more drug discovery tools implementing the drug discovery workflow.

As noted, in some example embodiments, the drug discovery ecosystem 100 may include the tool collection 130, which may include a variety of drug discovery tools including, for example, one or more computational models for molecule design generation and modification, molecular property prediction, candidate molecule ranking and selection, and/or the like. In some cases, the drug discovery tools in the tool collection 130 shown in FIG. 1 may be configured by one or more users interacting with the controller 110 of the drug discovery ecosystem 100. For example, in some cases, a builder (e.g., the builder 202 in FIG. 2A, the builder 305 in FIG. 3A) may contribute, for addition to the tool collection 130, one or more new drug discovery tools or, alternatively, one or more new capabilities to an existing tool in the tool collection 130. In some cases, the drug discovery tools in the tool collection 130 may be made available, for example, to one or more end users (e.g., the end user 204 in FIG. 2A, the end user 310 in FIG. 3B) in a drug discovery tool marketplace. In some cases, the drug discovery tool marketplace may include a user interface (UI) configured to display the available selection of drug discovery tools (e.g., in the tool collection 130). Furthermore, in some cases, the user interface (UI) associated with the drug discovery marketplace may be configured to receive one or more inputs selecting one or more drug discovery tools.

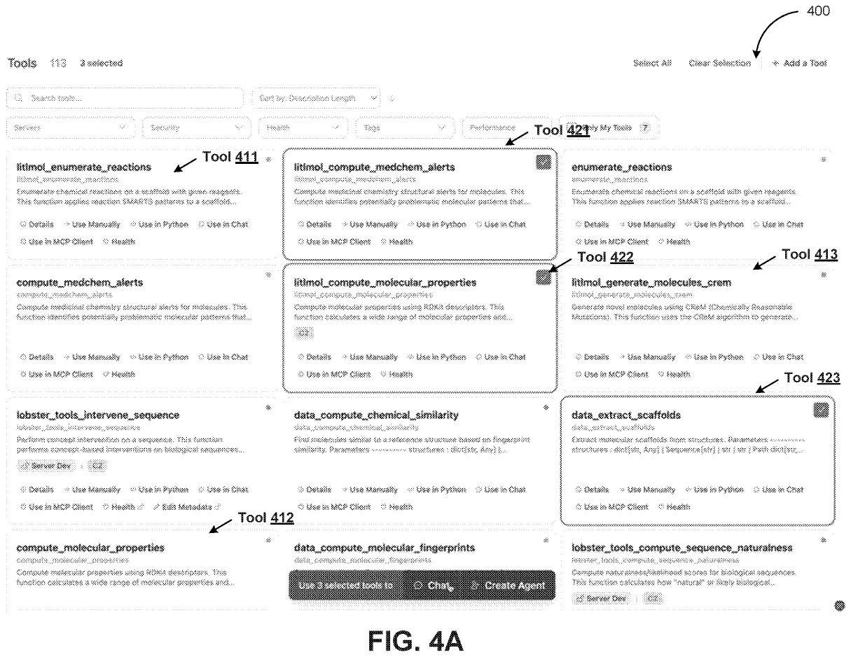

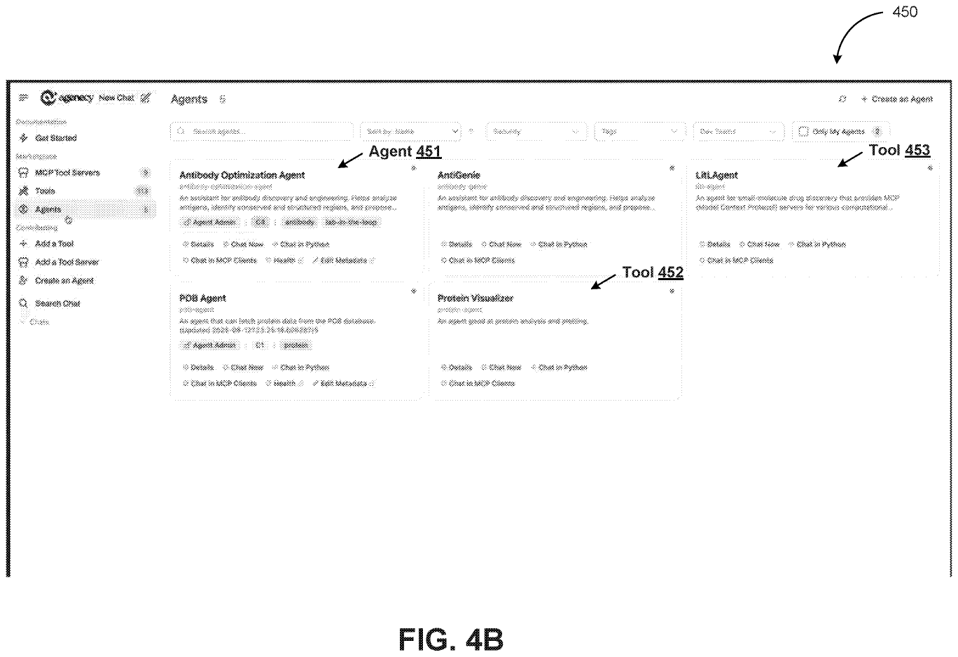

To further illustrate, FIG. 4A depicts a screenshot of an example of a user interface (UI) for a drug discovery tool marketplace 400, in accordance with some example embodiments. As shown in FIG. 4A, the user interface (UI) for the drug discovery tool marketplace 400 may display at least a portion of the available drug discovery tools (e.g., at least a portion of the drug discovery tools in the tool collection 130). In the example shown in FIG. 4A, each available drug discovery tool is display as a separate panel along with the name of the tool and a description of its functionalities. For example, in FIG. 4A, the drug discovery tool marketplace 400 may include a first tool 411 for enumerating chemical reactions on a scaffold with given agents, a second tool 412 for computing molecular properties using RDKit descriptors, and a third tool 413 for generating novel molecules using CReM (Chemically Reasonable Mutations). In some cases, the user interface (UI) for the drug discovery tool marketplace 400 may be configured to receive one or more inputs selecting one or more of the available drug discovery tools. For instance, FIG. 4A shows the selection (e.g., via a checkbox) of a fourth tool 421 for computing medicinal chemistry structure alerts for molecules, a fifth tool 422 for computing molecular properties using RDKit descriptors, and a sixth tool 423 for extracting molecular scaffolds from structures. As described in more detail below, in some cases, the one or more drug discovery tools selected from the drug discovery tool marketplace 400 may be triggered directly to perform the corresponding tasks. Alternatively, in some cases, the one or more drug discovery tools may be selected for inclusion in a drug discovery workflow orchestrated, in some cases, by an artificial intelligence (AI) agent.

It should be appreciated that the drug discovery tool marketplace 400 may include a wide range of drug discovery tools, including those leveraging computation models implementing using machine learning and/or deep learning architectures. In some cases, generative models proposing candidate molecule designs are one category of drug discovery tools. In some cases, these generative models may propose candidate molecule designs with implicit guidance, affinity-guided antibody maturation, a latent 3D generative approach for small molecule design, and/or the like. In some cases, property predictions models are another category of drug discovery tools. In some cases, property prediction models may complement the aforementioned generative models. In some cases, the property prediction models may include multiple property prediction models, including antibody developability assessment using molecular surface descriptors, antibody expression and antibody-antigen complex prediction, antibody profiling based on hydrophobicity and charge descriptors, and/or the like. In some cases, structural analysis methodologies are yet another category of drug discovery tools. In some cases, additional structural analysis methods may provide insights into molecular properties including, for example, ABangle for orientation characterization, PEP-Patch for electrostatic surface patch estimation, spatial aggregation propensity scoring for identifying aggregation-prone regions, and/or the like. In some cases, structural analysis methodologies may include those geared toward molecular docking and scoring. For example, in some cases, protein structures may be prepared using SPRUCE to ensure compatibility with docking pipelines. In some cases, ligand docking may be performed using POSIT, which generates ligand poses within binding pockets. In some cases, docking poses may be evaluated using HYBRID scoring, which integrates deep learning-based pose scoring with traditional docking methods. In some cases, metabolite prediction models may be applied to assist in evaluating small molecule modifications, identifying metabolic transformations of drug candidates and their corresponding probabilities.

FIG. 4B depicts a screenshot of an example of a user interface (UI) for an artificial intelligence (AI) agent marketplace 450, in accordance with some example embodiments. As shown in FIG. 4B, the user interface (UI) for the artificial intelligence (AI) agent marketplace 450 may display at least a portion of the available artificial intelligence (AI) agents in the drug discovery ecosystem 100. In the example shown in FIG. 4B, each available artificial intelligence (AI) agent is display as a separate panel along with the name of the agent and a description of its functionalities. For example, the selection of artificial intelligence (AI) agents in the example of the artificial intelligence (AI) agent marketplace 450 shown in FIG. 4B includes a first agent 451 that orchestrates a workflow for antibody discovery and engineering, a second agent 452 that orchestrates a workflow for protein analysis and plotting, and a third agent 453 that orchestrates a workflow for small-molecule drug discovery. In some cases, instead of selecting and triggering one or more of the drug discovery tools in the drug discovery tool marketplace 400 directly, an end user (e.g., the end user 204 in FIG. 2A, the end user 310 in FIG. 3B) may select one or more artificial intelligence (AI) agents from the artificial intelligence (AI) agent marketplace 450. In some cases, the selected artificial intelligence (AI) agent may orchestrate a corresponding drug discovery workflow including by triggering one or more constituent drug discovery tools.

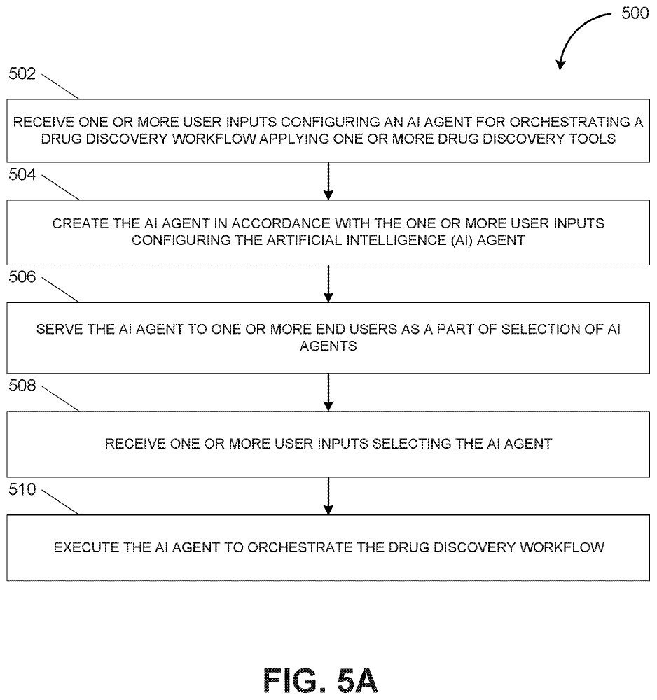

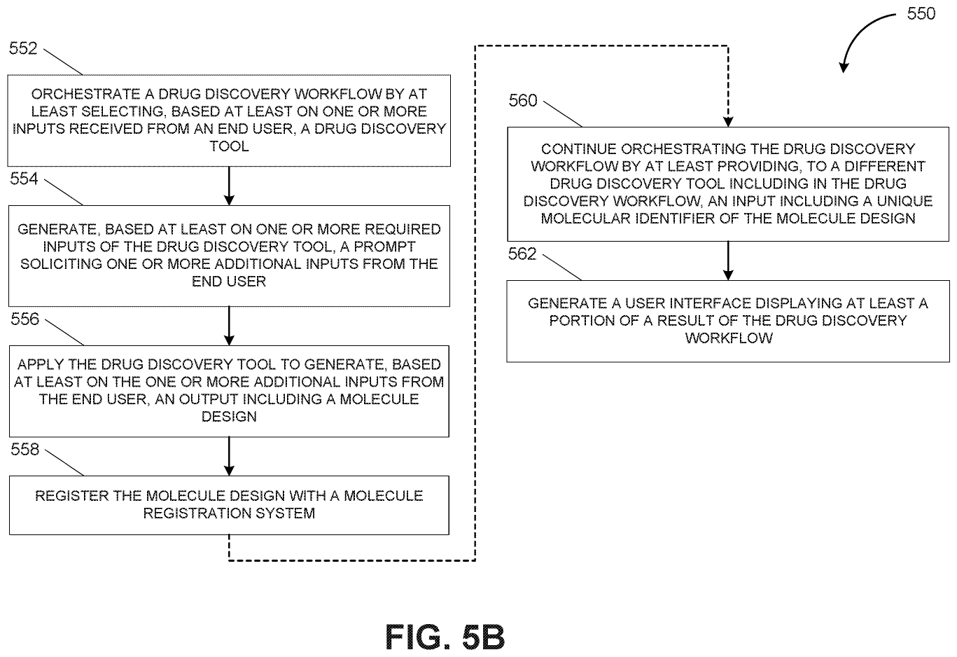

FIG. 5A depicts a flowchart illustrating an example of a process 500 for an artificial intelligence (AI) agent orchestrated drug discovery workflow, in accordance with some example embodiments. Referring to FIGS. 1-4 and 5A, in some example embodiments, the process 500 may be performed by the controller 110 to support the configuration of an artificial intelligence (AI) agent for orchestrating one or more drug discovery workflows. In some cases, the controller 110 may interface with a builder (e.g., the builder 202 in FIG. 2A, the builder 305 in FIG. 3A) contributing one or more new artificial intelligence (AI) agents, drug discovery tools, tool servers, drug discovery tool capabilities, and/or the like. For example, in some cases, the builder may interact with the controller 110 to configure an artificial intelligence (AI) agent to orchestrate a drug discovery workflow that applies one or more drug discovery tools, including new and/or existing drug discovery tools, from a drug discovery tool collection (e.g., the tool collection 130 in FIG. 1). As described in more detail below, in some cases, the artificial intelligence (AI) agent may be configured to interact with one or more end users to orchestrate a drug discovery workflow applying the one or more drug discovery tools. For instance, in some cases, the artificial intelligence (AI) agent may orchestrate the drug discovery workflow by at least coordinating the operations of the drug discovery tools included in the drug discovery workflow (e.g., the scheduling the drug discovery tools to be applied in an appropriate sequence and the transferring of input and output data therebetween) and interfacing with the one or more end users to obtain additional inputs required for continuing the drug discovery workflow.

At 502, one or more user inputs configuring an artificial intelligence (AI) agent for orchestrating a drug discovery workflow applying one or more drug discovery tools are received. In some example embodiments, a controller of a drug discovery ecosystem hosting a variety of drug discovery tools and artificial intelligence (AI) agents orchestrating drug discovery workflows applying these drug discovery tools may support the configuration additional artificial intelligence (AI) agents. For example, in some cases, the controller may receive, from a builder, one or more inputs designing and registering an artificial intelligence (AI) agent. In some cases, the controller may receive the one or more inputs via the user interface (UI) of a web-based application. In some cases, the controller may generate, for display at a client device of the builder, one or more user interfaces (UIs) for inputting specifications and prompts associated with the artificial intelligence (AI) agent. In some cases, the controller may also generate user interfaces (UI) for the builder to publish and further manage (e.g., permissions, edits, removal, and/or the like) the artificial intelligence (AI) agent.

As described in more detail below, the artificial intelligence (AI) agent may be configured to orchestrate a drug discovery workflow that includes the application of one or more drug discovery tools such as design computation models, property computation model, combinatorial optimization computation models, and/or the like. For instance, in some cases, the artificial intelligence (AI) agent may coordinate the operations of the drug discovery tools included in the drug discovery workflow such as scheduling the drug discovery tools to be applied in an appropriate sequence and transferring of input and output data between different drug discovery tools. Furthermore, in some cases, the artificial intelligence (AI) agent may interface with the one or more end users to obtain additional inputs required for continuing the drug discovery workflow.

At 504, the artificial intelligence (AI) agent is created in accordance with the one or more user inputs configuring the artificial intelligence (AI) agent. In some example embodiments, the controller of the drug discovery ecosystem may create the artificial intelligence (AI) agent at a centralized agent server. For example, in some cases, one or more configuration files of the artificial intelligence (AI) agent may be pushed to a repository associated with the drug discovery ecosystem. In some cases, centralized agent server may adopt one or more agent serving standards such as, for example, OpenAI chat completion standard Representational State Transfer (REST) application programming interface (API), Model Context Protocol (MCP) and/or the like. In this context, an agent serving standard may govern the building, deployment, management, and collaboration of artificial intelligence (AI) agents.

In some cases, the centralized agent server may further provide the infrastructure, frameworks, runtimes, registries, and marketplaces to support the building, deployment, management, and collaboration of artificial intelligence (AI) agents. In some cases, the controller may create the artificial intelligence (AI) agent at the centralized agent server by at least compiling an executable instance of the artificial intelligence (AI) agent running in memory from the configurations specified in the one or more inputs received from the builder. In some cases, the controller may further provide storage for user state and memory, chat endpoint for each conversation turns' response, and a task queue to support the execution of the artificial intelligence (AI) agent.

At 506, the artificial intelligence (AI) agent is served to one or more end users as a part of a selection of artificial intelligence (AI) agents. In some example embodiments, once the controller creates and registers the artificial intelligence (AI) agent at the centralized agent server, the artificial intelligence (AI) agent may be made available for user by one or more end users of the drug discovery ecosystem. For example, in some cases, the artificial intelligence (AI) agent may be added to an agent marketplace, thus exposing the artificial intelligence (AI) agent to the one or more end users. In some cases, the one or more end user of the drug discovery ecosystem may select the artificial intelligence (AI) agent, from the selection of artificial intelligence (AI) agents made available through the agent marketplace, to orchestrate a corresponding drug discovery workflow.

At 508, one or more user inputs selecting the artificial intelligence (AI) agent is received. In some example embodiments, the controller may generate, for display at a client device of an end user, a user interface (UI) depicting a selection of artificial intelligence (AI) agents available from the drug discovery ecosystem (e.g., a gallery or menu of artificial intelligence (AI) agents). In some cases, the selection of artificial intelligence (AI) agents may include the artificial intelligence (AI) configured by the builder in operation 502. In some cases, the controller may receive, via the user interface (UI) at a client device associated with an end user, one or more inputs selecting the artificial intelligence (AI) agent from the selection of artificial intelligence (AI) agents in the artificial intelligence (AI) agent marketplace.

At 510, the artificial intelligence (AI) agent is executed to orchestrate the drug discovery workflow. In some example embodiments, the controller may respond to the one or more inputs from the end user selecting the artificial intelligence (AI) agent by at least executing the artificial intelligence (AI) agent. In some cases, the executing of the artificial intelligence (AI) agent may include launching the artificial intelligence (AI) agent. In some cases, launching the artificial intelligence (AI) agent may include initiating a chat with the end user. For example, in some cases, the artificial intelligence (AI) agent may prompt the end user to provide one or more inputs specifying one or more objectives of a drug discovery workflow for orchestration by the artificial intelligence (AI) agent. As described in more detail below, the end user may interact with the artificial intelligence (AI) agent in order to execute the drug discovery workflow. For instance, in some cases, the end user may interact with the artificial intelligence (AI) agent using natural language queries to provide the necessary inputs to the drug discovery tools implementing the drug discovery workflow. In some case, the artificial intelligence (AI) agent may prompt the end user for additional inputs where necessary.

FIG. 5B depicts a flowchart illustrating an example of a process 550 for an artificial intelligence (AI) agent orchestrated drug discovery workflow, in accordance with some example embodiments. Referring to FIGS. 1-4 and 5A-B, in some example embodiments, the process 550 may be performed by an artificial intelligence (AI) agent (e.g., the one or more agents 120 in FIG. 1) to orchestrate one or more corresponding drug discovery workflows. In some cases, the process 550 may implement operation 510 of the process 500 shown in FIG. 5A. For example, in some cases, the controller 110 may interface with an end user (e.g., the end user 204 in FIG. 2A, the end user 310 in FIG. 3B) including by generating a user interface (UI) that displays, at a client device of the end user (e.g., the user interface 165 at the client device 160), an artificial intelligence (AI) agent marketplace exposing a selection of artificial intelligence (AI) agents available from the drug discovery ecosystem 100. In some cases, the end user may interact with the controller 110 to select an artificial intelligence (AI) agent to orchestrate a corresponding drug discovery workflow. As described in more detail below, executing the artificial intelligence (AI) agent may include performing the drug discovery workflow, which may include the application of one or more drug discovery tools, for example, from a drug discovery tool collection (e.g., the tool collection 130 in FIG. 1). In some cases, the artificial intelligence (AI) agent may interact with the end user using, for example, natural language based queries. Moreover, in some cases, the artificial intelligence (AI) agent may orchestrate the drug discovery workflow by at least coordinating the operations of the drug discovery tools included in the drug discovery workflow (e.g., the scheduling the drug discovery tools to be applied in an appropriate sequence and the transferring of input and output data therebetween) and interfacing with the one or more end users to obtain additional inputs required for continuing the drug discovery workflow.

At 552, an artificial intelligence (AI) agent orchestrates a drug discovery workflow by at least selecting, based at least on one or more inputs received from an end user, a drug discovery tool. In some example embodiments, a drug discovery ecosystem may include a controller that generates, for display at a client device of an end user, a user interface (UI) for selecting the artificial intelligence (AI) agent to orchestrate the drug discovery workflow. For example, in some cases, the user interface (UI) may display a gallery (or menu) of available artificial intelligence (AI) agents and a description of its functionalities. In some cases, the drug discovery workflow may be a large molecule drug discovery workflow for designing therapeutic proteins or peptides, a small molecule drug discovery workflow for designing small molecule drug compounds, a conjugate drug discovery workflow for designing molecules combining multiple modalities, a gene therapy drug discovery workflow for designing therapeutic nucleic acids, and/or the like. In some cases, the drug discovery workflow may be a predetermined drug discovery workflow that invokes a fixed set of drug discovery tools in a preset sequence. Alternatively, the drug discovery workflow may be an adaptive drug discovery workflow in which the artificial intelligence (AI) agent dynamically selects the drug discovery tools based on its interaction with the end user and the inputs received therefrom.

In some example embodiments, in response to the end user selecting the artificial intelligence (AI) agent, the controller may launch the artificial intelligence (AI) agent such that the end user may interact with the artificial intelligence (AI) agent using natural language queries. For instance, in some cases, the end user may send, to the artificial intelligence (AI) agent, a natural language query specifying one or more objectives for the drug discovery workflow. In some cases, the artificial intelligence (AI) agent may be trained to identify, based at least on the natural language query, at least one drug discovery tool (e.g., from the available drug discovery tools) for accomplishing the one or more objectives of the drug discovery workflow specified by the end user. In some cases, the at least one drug discovery tool may be a molecule design computation model trained to generate one or more molecule designs. In some cases, the artificial intelligence (AI) agent may orchestrate the drug discovery workflow by at least applying the molecule design computation model to generate one or more molecule designs before applying one or more additional computation models to evaluate, rank, and/or further optimize the molecule designs.

At 554, the artificial intelligence (AI) agent generates, based at least on one or more required inputs of the drug discovery tool, a prompt soliciting one or more additional inputs from the end user. In some example embodiments, the artificial intelligence (AI) agent may orchestrate the drug discovery workflow by at least soliciting one or more inputs from the end user where necessary. For example, in instances where the drug discovery tool selected by the artificial intelligence (AI) agent in operation 552 is enabled by data that is absent from the one or more inputs received from the end user, the artificial intelligence (AI) agent may generate a prompt soliciting the data from the end user. In some cases, the prompt soliciting the one or more additional inputs from the end user displays one or more preconfigured inputs for the drug discovery tool.=

At 556, the artificial intelligence (AI) agent applies the drug discovery tool to generate, based at least on the one or more additional inputs from the end user, an output including a molecule design. In some example embodiments, the drug discovery tool selected by the artificial intelligence (AI) agent in operation 552 may include a molecule design computation model trained to generate one or more molecule designs. For example, in some cases, the molecule design computation model may be trained to generate one or more novel small molecule drug compounds, therapeutic proteins or peptides, nucleic acids, and/or the like. In some cases, the molecule design computation model may generate the one or more molecule designs by operating on molecular embeddings. For instance, in some cases, the molecule design computation model may generate the one or more molecule designs by operating on the molecular embedding of a seed molecule design. In some cases, the molecule design computation model may operate on molecular embeddings generated by a universal molecular encoder (UME) trained to generate embeddings of multiple modalities of molecules including, for example, small molecule drug compounds, therapeutic peptides and proteins, nucleic acids, and complexes thereof.

At 558, the artificial intelligence (AI) agent registers the molecule design with a molecule registration system. In some example embodiments, the molecule registration system may provide centralized and uniform access to data associated with a multitude of molecule designs including, for example, chemical formulation, amino acid sequence, three-dimensional structure and/or substructure, properties, lineage, and/or the like. Accordingly, in some cases, registering the molecule design with the molecule registration system may facilitate further operations on the molecule design throughout the remainder of the current drug discovery workflow as well as subsequent efforts to further develop the molecule design in both the dry and web lab. For example, in some cases, instead of transferring the data associated with the molecule design between the different drug discovery tools implementing the drug discovery workflow, which consumes bandwidth and increases latency, this data may be accessible to each drug discovery tool from a central repository (e.g., a relational database) based on a unique, persistent molecule identifier assigned to the molecule design by the molecule registration system. For instance, in some cases, the data associated with the molecule design generated by the molecule design computation model may be uploaded to the molecule registration system and stored in the central repository (e.g., the relational database) along with a corresponding molecule identifier. As described in more details, in some cases, one or more other drug discovery tools in the drug discovery workflow may subsequently access at least a portion of the data associated with the molecule design using the molecular identifier associated with the molecule design.

In some example embodiments, the molecule registration (TMR) system may store the molecule design in accordance with a hierarchical data model that is agnostic to the modality of the molecule design. For example, in some cases, the hierarchical data model may support the storage of small molecule drug compounds and large molecules (e.g., therapeutic peptides and proteins). In some cases, by registering the molecule design with the molecule registration system, the molecule design may be assigned a unique system identifier. Furthermore, in some cases, the molecule design may be assigned a semantic identifier with semantic significance to the end user. In some cases, in accordance with the hierarchical data model, the system identifier and the semantic identifier of the molecule design may include a concept identifier of a concept corresponding to a conceptualized version of the corresponding molecule. In some cases, where the molecule design is subsequently synthesized, each batch of the synthesized molecule design may be assigned a unique system identifier and semantic identifier having a corresponding batch identifier. In some cases, the molecule design may be stored by the molecule registration system as multiple constituent components. For instance, where the molecule design is a small molecule drug compound, the molecule design may be stored as a core structure (e.g., the active drug molecule) and the corresponding salt form (e.g., formed by reacting the core structure with an acid or base to yield a neutral, crystalline structure with improved properties for drug delivery), each of which being associated with a unique system identifier and semantic identifier. Alternatively, where the molecule design is a protein molecule (e.g., a therapeutic protein such as an antibody), the molecule design may be stored as a heavy chain and a light chain, each of which being associated with unique system identifier and semantic identifier. In some cases, the molecule design may be stored at a central repository implemented using a relational database in which data associated with a molecule design, including the constituent components of the molecule and one or more batches of the synthesized molecule design, are stored across one or more tables linked by keys (e.g., primary keys, foreign keys, and/or the like).

At 560, the artificial intelligence (AI) agent continues orchestrating the drug discovery workflow by at least providing, to a different drug discovery tool included in the drug discovery workflow, an input including a unique molecular identifier of the molecule design. In some example embodiments, the artificial intelligence (AI) agent may orchestrate the drug discovery workflow by transferring data between the drug discovery tools implementing the drug discovery workflow. For example, in some cases, where one drug discovery tool in the drug discovery workflow is a molecule design computation model that is applied by the artificial intelligence (AI) agent to generate the molecule design, a subsequent drug discovery tool in the drug discovery workflow may be a property computation model trained to determine one or more properties of the molecule design. In some case, yet another drug discovery tool in the drug discovery workflow may be a selection computation model trained to rank two or more molecule designs based on individual or multiple properties of interest. In some cases, the input provided to the subsequent drug discovery tool in the drug discovery workflow may include data associated with the molecule design. In some cases, instead of the data itself, the artificial intelligence (AI) agent may provide the unique molecular identifier of the molecule design such that the subsequent drug discovery tool may retrieve, from the central repository of the molecule registration system, the necessary data based on the unique molecular identifier of the molecule design. In some cases, the subsequent drug discovery tool, such as the property computation model, may operate on an embedding of the molecule design generated by a universal molecular encoder (UME) trained to generate embeddings of multiple modalities of molecules including, for example, small molecule drug compounds, therapeutic peptides and proteins, nucleic acids, and complexes thereof.

In some example embodiments, the artificial intelligence (AI) agent may identify the subsequent drug discovery tool for application in the drug discovery workflow. For example, in some cases, the drug discovery workflow may be a predetermined drug discovery workflow that invokes a fixed set of drug discovery tools in a preset sequence. In some cases, where the drug discovery workflow is a predetermined drug discovery workflow, the next drug discovery tool in the preset sequence of the drug discovery workflow may be identified by the artificial intelligence (AI) agent as the subsequent drug discovery tool for application in the drug discovery workflow. Alternatively, in some cases, the drug discovery workflow may be an adaptive drug discovery workflow without a fixed set of drug discovery tools or without a preset sequence for applying the drug discovery tools. In some cases, where the drug discovery workflow is an adaptive drug discovery workflow, the artificial intelligence (AI) agent may identify the subsequent drug discovery tool based at least on one or more inputs received from the end user. In some cases, the subsequent drug discovery tool may be identified based at least on the one or more objectives specified by the end user in the initial natural language query received from the end user. In some cases, the subsequent drug discovery tool may also be determined based at least on one or more additional inputs received from the end user during the execution of the drug discovery workflow orchestrated by the artificial intelligence (AI) agent. In some cases, the sequence of drug discovery tools applied during the execution of the drug discovery workflow may corresponding to a trajectory (or chain) of actions determined based on an intention of the end user as determined based on one or more inputs from the end user and a pool of reference intention-action pairs. For instance, in some cases, one or more additional inputs received from the end user may render one drug discovery tool more suitable than other drug discovery tools in the drug discover tool collection, in which case the artificial intelligence (AI) agent may identify that drug discovery tool as the subsequent drug discovery tool for application in the drug discovery workflow. In some cases, instead of applying the subsequent drug discovery tool, the artificial intelligence (AI) agent may determine that the output from one or more previous applications of the drug discovery tool may be reused, in which case the artificial intelligence (AI) agent may continue orchestrating the drug discovery workflow by reusing those outputs instead of applying the subsequent drug discovery tool.

In some example embodiments, instead of multiple computation models trained to determine individual properties of interest, the subsequent drug discovery tool in the drug discovery workflow may be a multi-property computation model trained to determine multiple properties present in the molecule design including, for example, drug-like properties such as binding affinity, specificity, biological activity, developability, and/or the like. For example, in some cases, the multi-property computation model may be trained to perform multiple tasks, each of which corresponding to the prediction of a property of interest. Furthermore, in some cases, the multi-property computation model may also be trained to be multi-modal, meaning that it is capable of determining the properties of interest of multiple molecular modalities including small molecule drug compounds, therapeutic peptides and proteins, nucleic acids, and/or the like. In some cases, the multi-property computation model may be trained to determine one or more properties of interest based on a molecular representation of the molecule design as well as contextual information. Examples of contextual information may include descriptions of the experimental environment in which the properties of interest are measured. In some cases, contextual information may be better described in natural language. Accordingly, in some cases, the multi-property computation model described herein may be implemented using a language model (e.g., a large language model) trained to operate on natural language inputs. Moreover, in some cases, the multi-property computation model may be a reasoning model (e.g., a reasoning language model (RLM)) trained to decompose the complex task of determining multiple properties of interest into multiple constituent operations. For instance, in some cases, the multi-property computation model may explore different sequences of operations for determining the properties of interest. In some cases, upon exploring different sequences of operations, at least a portion of the one sequence of operations that is selected by the multi-property computation model may be provided (e.g., as a natural language based output) to one or more end users of the drug discovery ecosystem along with a result of performing the sequence of operations (e.g., values for one or more properties of interest present in a molecule design). In some cases, the sequence of operations that lead to the result may correspond to the reasoning process partook by the property computation model in order to reach its conclusions (or outcomes). In some cases, implementing the multi-property computation model as a reasoning model such that its reasoning process are available to the end users may increase the explainability and interpretability of the multi-property computation model. In this context, the explainability of the multi-property computation model may refer to the ability to provide semantically meaningful rationale for the output (e.g., values of the one or more properties of interest) generated by the multi-property computation model. In some cases, the interpretability of the multi-property computation model may refer to the degree to which the internal mechanisms (e.g., weights, biases, and/or the like) of model are semantically meaningful.

In some example embodiments, the multi-property computation model may be trained to optimize (e.g., reduce or minimize) an objective function with one or more outcome terms (e.g., outcome rewards) that aims to reduce (or minimize) the discrepancy between the properties of interest determined by the multi-property computation model and the corresponding ground-truth values. Alternatively and/or additionally, in some cases, the multi-property computation model may be trained to optimize (e.g., reduce or minimize) an objective function with one or more process terms (e.g., process rewards) that aims to reduce (or minimize) the discrepancy between the sequences of operations performed by the multi-property computation model to determine the properties of interest and the corresponding ground-truth sequences of operations.

At 562, the artificial intelligence (AI) agent generates a user interface (UI) displaying at least a portion of a result of the drug discovery workflow. In some example embodiments, the result of the drug discovery workflow may be determined based on the outputs from one or more of the drug discovery tools included in the drug discovery workflow. For example, in some cases, the result of the drug discovery workflow may include one or more molecule designs satisfying the objectives specified in the initial inputs provided by the end user.

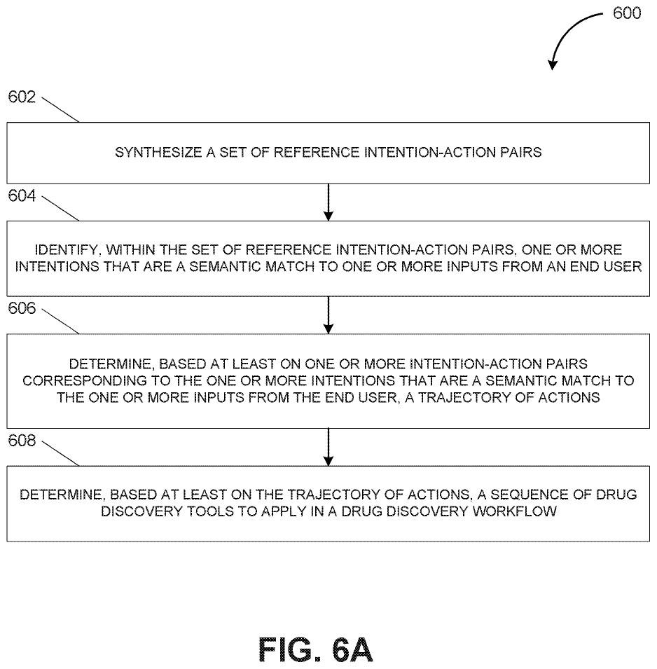

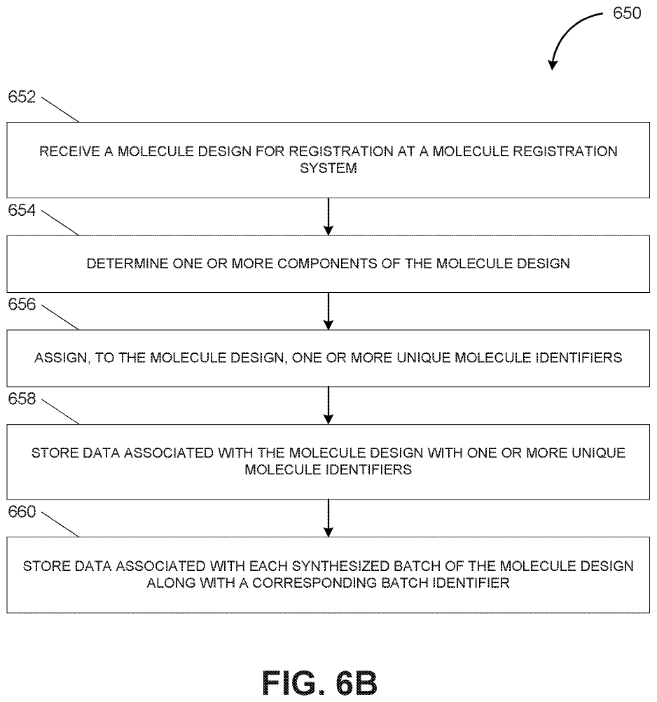

FIG. 6A depicts a flowchart illustrating an example of a process for intention-aware determination of action sequences in a drug discovery workflow, in accordance with some example embodiments. Referring to FIGS. 1-5 and 6A, in some cases, the process 600 may be performed by the one or more agents 120 in the drug discovery ecosystem 100. In some cases, the process 600 may be performed to dynamically determine a drug discovery workflow based on one or more inputs from an end user of the drug discovery ecosystem 100. Alternatively, in some cases, the process 600 may implement at least a portion of operations 552 and 560 in which an artificial intelligence (AI) agent orchestrates a drug discovery workflow by identifying a subsequent drug discovery tool to apply in the drug discovery platform. As described in more detail below, the process 600 may be performed with scientific intention awareness such that the drug discovery tools in the drug discovery workflow are applied in a sequence that is consistent with the intentions of the end user. For example, in some cases, the one or more agents 120 may be trained to map high-level and ambiguous scientific intention to actional uses of drug discovery tools.

At 602, a set of reference intention-action pairs is synthesized. In some example embodiments, an artificial intelligence (AI) agent may plan a trajectory of actions that is consistent with an end user's scientific intentions and identify one or more suitable drug discovery tools for each action in the trajectory of actions. In some cases, a primary artificial intelligence (AI) agent may perform the action planning while one or more specialized artificial intelligence (AI) agents may be deployed to handle the interactions (e.g., using natural language based queries) between the end user and one or more of the drug discovery tools involved in the drug discovery workflow. In some cases, due to the expansive and ever expanding nature of the selection of available drug discovery tools in a drug discovery ecosystem, the end user may be unable to formulate concrete action plans. Accordingly, in some cases, an artificial intelligence (AI) agent, such as a primary artificial intelligence (AI) agent, may create the action plans underlying a drug discovery workflow by at least determining the intentions of the end user from the end user's inputs (e.g., natural language queries), determining the potential sub-operations, requesting clarification and additional inputs from the end user, and navigating the available resources at the drug discovery ecosystem. As described in more detail below, a pool of initial intentions and corresponding action chains may be synthesized and used as reference for the artificial intelligence (AI) agent to bridge the intention-to-action gap.

In some cases, in the absence of existing data that includes drug discovery intentions and corresponding actions to realizing them. As such, in some cases, to obtain a reasonable quantity of intention-action pairs without expensive expert annotation, a self-play agent may be deployed to produce both intention and the corresponding action chains. In some cases, according to the input and output specification defined as parts of the drug discovery tools, a set of valid action chains may be curated where the upstream drug discovery tool's output datatype corresponds to the downstream drug discovery tool's input datatype. In some cases, potential intention that leads to an action chain with a self-play agent may be reverse generated using, for example, a language model (e.g., large language model (LLM)). In some cases, the actions may be coupled with related descriptions to enrich the context of the considered steps in the action chains, in addition to a few similar action chains as negative examples to guide the language model (e.g., large language model (LLM)) in generating an intention that applies to the target positive action chain.

At 604, one or more intentions that are a semantic match to one or more inputs from an end user are identified within the set of reference intention-action pairs. In some example embodiments, when the artificial intelligence (AI) agent (e.g., the primary artificial intelligence (AI) agent responds to an input (e.g., natural language query) from the end user, one or more intentions in the reference pool that semantically match the input are identified.

At 606, a trajectory actions is determined based at least on one or more intention-action pairs corresponding to the one or more intentions that are the semantic match to the one or more inputs from the end user. In some example embodiments, the one or more intentions in the reference pool that semantically match the input (e.g., natural language query) from the end user as well as their corresponding action chains may be appended as part of the conversation history between the artificial intelligence (AI) agent (e.g., the primary artificial intelligence (AI) agent) and the end user. In some cases, these references may be added before any tool-calling and reasoning by the artificial intelligence (AI) agent (e.g., primary artificial intelligence (AI) agent) such that the artificial intelligence (AI) agent is able to leverage these references when determining a suitable action chain.

At 608, a sequence of drug discovery tools to apply in a drug discovery workflow is determine based at least on the trajectory of actions. In some example embodiments, the sequence of drug discovery tools may include one or more computation models for molecule design generation and modification, molecular property prediction, candidate molecule ranking and selection, structural analysis, and/or the like. In some cases, while the primary artificial intelligence (AI) agent is tasked with planning the drug discovery workflow, including the sequence of drug discovery tools that are applied, one or more specialized artificial intelligence (AI) agents may participate in the execution of the drug discovery workflow. In some cases, the separation of agents for the planning of drug discovery workflows and those specializing in the execution of the drug discovery workflows may increase the scalability of the drug discovery ecosystem. For example, when new capabilities are added to existing drug discovery tools or new drug discovery tools are added to the drug discovery ecosystem, no additional context window is taken for the primary artificial intelligence (AI) agent as the model information is obtained through the metadata-indexed searching tools. In some cases, when responding to queries directed to a specific drug discovery tool (e.g., inquiries about the training data being used and the evaluation performance of the drug discovery tool) or when a specific drug discovery tool is executed, these capability-specific tasks are done by the corresponding specialized artificial intelligence (AI) agents.

In some cases, sophisticated drug discovery tools, such as those leverage computation models (e.g., machine learning and deep learning models), may trigger a complicated sequence of actions including the acquiring, processing, and validating inputs, data loading, and asynchronous execution in virtual machines. In some cases, a specialized artificial intelligence (AI) agent may be created for each drug discovery tool with a separate system role and access to the tool-specific resources. For example, in some cases, a specialized artificial intelligence (AI) agent may be created for each of a molecule design computation model, a property computation model, and a selection computation model in a drug discovery workflow. In some cases, the specialized artificial intelligence (AI) agent may be instructed to prompt an end user to provide the necessary input data and validate the input data. In some cases, once the input data is validated, the specialized artificial intelligence (AI) agent may call a launch tool to launch a script on a virtual machine with the validated input data. In some cases, the specialized artificial intelligence (AI) agent associated with a drug discovery tool may further route the execution of the drug discovery workflow back to the primary artificial intelligence (AI) agent when the drug discovery tool completes its task or when the specialized artificial intelligence (AI) agent determines that the task is outside of the scope of the drug discovery tool.

In some cases, the specialized artificial intelligence (AI) agent may have access to existing question-answer pools containing detailed questions about the specific capabilities of the corresponding drug discovery tool. In some cases, at least a portion of these questions and answers may be generated by a language model (e.g., a large language model (LLM)) based on the source paper or documentation describing the details of the drug discovery tool. In some cases, a separate index for questions may be generated instead of retrieving semantically similar paragraphs from the source paper and/or documentation directly, considering the semantic space of the questions is more similar to the potential user queries about the model.

In some cases, after the primary assistant has confirmed the action plan, the execution may be performed by the specialized artificial intelligence (AI) agents associated with each constituent drug discovery tool. In some cases, the primary artificial intelligence (AI) agent may elect to route to one of the specialized artificial intelligence (AI) agents once an action plan is created. In some cases, a proxy tool node for each specialized artificial intelligence (AI) agent may be created and bonded to the primary artificial intelligence (AI) agent such that each tool calling would route the agent flow to the corresponding specialized artificial intelligence (AI) agent. In this context, “agent flow” may refer to the passing of the orchestration of the drug discovery workflow between the primary and one or more specialized artificial intelligence (AI) agents during its execution. In some cases, when the current state is in a specialized artificial intelligence (AI) agent, the router may choose to exit out of it and route back to the primary artificial intelligence (AI) agent if the specialized artificial intelligence (AI) agent detects that the user's query is beyond its scope.

In some cases, one or more hint nodes may be available to balance controlled flow and flexibility. In some cases, dynamic instructions to one or more artificial intelligence (AI) agents may emerge on the fly depending on the outcome of upstream conversations. However, including all instructions in the static system role may not feasible. In some cases, defining a fixed routing graph may limit the generalizability and flexibility of the agents. Accordingly, in some cases, one or more hint routing nodes may be provided to guide the agents with dynamic instructions. For example, in some cases, the dynamic instructions may be appended by appending system turns to the conversation history with a reminder message. In some cases, the hint node may be implemented as a routing node for the agent. For instance, if the certain condition is met, then a hint node may add a hint to a conversation turn. In some cases, the use of hints nodes may enable the agents to be guided, for example, to verify critical actions, stay consistent with the action plan, and handle hallucinated values (e.g., from one or more language models), while maintaining the flexibility enabled by the strong reasoning skills of the underlying language models (e.g., large language models (LLMs)).

In some example embodiments, the hint node may ensure that an agent confirms that an end user is satisfied with the produced action plan before engaging specialized artificial intelligence (AI) agents to prepare input data and execute the corresponding drug discovery workflow. In some cases, prior to launching the execution jobs given input data from the end user, the specialized artificial intelligence (AI) agent may confirms the inputs are correct. In some cases, both confirmations may be achieved prior to engaging the specialized artificial intelligence (AI) agents associated with the drug discovery workflow with the addition of hint nodes to remind the primary artificial intelligence (AI) agent to obtain such confirmations.

In some example embodiments, since the execution of each drug discovery tool may potentially trigger dozens of turns with the end user to solicit input, receive the input, validate the input, and launch the corresponding job, it is possible for the primary artificial intelligence (AI) agent to lose track of the remaining actions in the action chain. Accordingly, in some cases, a hint node reminding the agent of the action plan in the state may be added after routing back to the primary artificial intelligence (AI) agent from a specialized one. In some cases, the action plan may be saved as a natural language sentence right before entering any specialized artificial intelligence (AI) agents. In some cases, in the hint message, the action plan may be retrieved from the tracked state and include as a part of the pseudo-reminder messages saved to memory.

In some example embodiments, after receiving one or more inputs from an end user (e.g., the heavy and light chain sequences of an antibody), those values may be saved to the state memory of the agent. In some cases, if the input to a drug discovery tool is not a part of the input from the end user, it may be hallucinated by one or more language models (e.g., large language models (LLMs)) involved in the drug discovery workflow if the end user does not explicitly ask the other drug discovery tool to generate candidate values from scratch. In some cases, a hallucination handling hint node may be added before applying the drug discovery tool to remind the agent to confirm the source of input values and potentially correct values with unknown sources.

FIG. 6B depicts a flowchart illustrating an example of a process 650 for universal molecule registration, in accordance with some example embodiments. Referring to FIGS. 1-5 and 6A, in some cases, the process 650 may be performed by the molecule registration system 140 including, for example, the registration engine 142. In some cases, one or more molecule designs generated as a result of a drug discovery workflow may be registered with the molecule registration system 140. In some cases, registering a molecule design with the molecule registration system 140 may include the molecule design being assigned a unique molecular identifier including, for example, a system identifier and a semantic identifier having semantic significance to end users of the drug discovery ecosystem 100. As described in more detail below, in some cases, the molecule registration system 140 may provide centralized and uniform access to data associated with the one or more molecule designs, such as chemical formulation, amino acid sequence, three-dimensional structure and/or substructure, properties, lineage, and/or the like. For example, in some cases, a molecule design may be associated with a concept corresponding to a conceptualized version of the corresponding molecule. In some cases, the conceptualized version of a molecule may be defined based on its molecular composition, molecular structure, and/or the like. For instance, in some cases, the conceptualized version of a molecule design may be represented by one or more text-based notations such as a Simplified Molecular Input Line Entry System (SMILES) string or an International Chemical Identifier (InChl) for a small molecule drug compound or an amino acid residue sequence or a hierarchical editing language for macromolecules (HELM) string for a protein molecule. In some cases, the concept of the molecule design may be assigned a unique concept identifier. In some cases, a molecule registration system may store the molecule design as two or more individual components of the molecule design (e.g., the core structure and the salted form of a small molecule drug compound, the heavy and light chains of an antibody, and/or the like), each of which being associated with the concept of the molecule design. In some cases, in instances where the molecule design is synthesized, each batch of the synthesized molecule design may be associated with concept of the molecule design.

At 652, a molecule design is received for registration at a molecule registration system. In some example embodiments, the molecule registration system may be configured to provide centralized and uniform access to data associated with multiple modalities of molecules. Accordingly, in some cases, the molecule design received for registration at the molecule registration system may be a small molecule drug compound, a therapeutic protein or peptide, a nucleic acid, or a conjugate of multiple molecular modalities.

At 654, one or more components of the molecule design are determined. In some example embodiments, the molecule registration system may store a single molecule design as multiple separate components. In some cases, where the molecule design is a small molecule drug compound, the individual components of the molecule design may include a core structure (e.g., the active drug molecule) and the corresponding salt form, the latter being the result of reacting the core structure with an acid or base to yield a neutral, crystalline structure with improved properties for drug delivery. In some cases, the core structure of the molecule design may be further decomposed into two or more constituent substructures (or fragments). For example, the small molecule drug compound citalopram may be associated with the core structure C₂₀H₂₁FN₂O and the salt form C₂₀H₂₁FN₂O·HBr. In some cases, where the molecule design is a therapeutic protein, such as an antibody, the components of the molecule design may include the antigen binding fragment (Fab) and crystallizable fragment (Fc), the heavy chain and light chain, the framework (or constant) region and variable region, one or more complementarity determining regions (CDRs), and/or the like.

At 656, one or more unique molecule identifiers are assigned to the molecule design. In some example embodiments, the molecule registration system may implement a hierarchical data model that is agnostic to the modality of the molecule design. For example, in some cases, the hierarchical data model may associate each molecule design (and its constituent components) with a concept. In some cases, the concept of a molecule design may correspond to a conceptualized version of the corresponding molecule. In some cases, the concept of the molecule design may be represented by one or more text-based notations such as a Simplified Molecular Input Line Entry System (SMILES) string or an International Chemical Identifier (InChl) for a small molecule drug compound or an amino acid residue sequence or a hierarchical editing language for macromolecules (HELM) string for a protein molecule. In some cases, the concept of the molecule design may be associated with a concept identifier. In some cases, the concept of the molecule design may serve as a nexus linking components of the molecule design and synthesized batches of the molecule design within the molecule registration system. For instance, in some cases, the concept identifier may be used to link various data associated with the molecule design, such as data associated with individual components of the molecule design and data associated with synthesized batches of the molecule design.

At 658, data associated with the molecule design is stored with the one or more unique molecule identifiers. In some example embodiments, the registration of the molecule design at the molecule registration system may include the registration engine of the molecule registration system assigning, to the molecule design, a molecule identifier that includes a unique system identifier differentiating the molecule design from other molecule designs registered within the molecule registration system. In some cases, the registration engine may further assign, to the molecule design, a unique semantic identifier which, unlike the system identifier, has semantic significance to one or more end users of the drug discovery ecosystem. In some cases, the registration engine may maintain a mapping between the system identifier of the molecule and one or more semantic identifiers of the molecule design. For example, in some cases, different constituent components of the molecule design and any synthesized batches of the molecule design may be linked by the concept of the molecule design. As noted, in some cases, the concept of the molecule design may correspond to a conceptualized version of the corresponding molecule represented, for example, by one or more text-based notations such as a Simplified Molecular Input Line Entry System (SMILES) string or an International Chemical Identifier (InChl) for a small molecule drug compound or an amino acid residue sequence or a hierarchical editing language for macromolecules (HELM) string for a protein molecule.

In some example embodiments, the concept of the molecule design may be associated with a unique concept identifier. In some cases, the unique concept identifier of the molecule design may link the data associated with the concept itself (e.g., the one or more text-based notations representative of the concept of the molecule design) with the data associated with the individual components (e.g., structures, substructures, and/or the like) of the molecule design. Furthermore, in some cases, the unique concept identifier of the molecule design may further link the data associated with each synthesized batch of the molecule design. For example, in some cases, the molecule registration system may include a relational database in which the data associated with different molecule designs are stored across two or more tables. In some cases, the two or more tables may be linked by keys such as primary keys that uniquely identifies each record (or row) in a table and foreign keys that references the primary keys stored in another table to establish a link between the corresponding records (or rows). For example, in some cases, the concept identifier of the molecule design may serve as a foreign key that links records (or rows) in one or more tables storing the constituent components (e.g., structures, substructures, and/or the like) of the molecule design with the corresponding records in another table storing synthesized batches of molecule designs. In some cases, data associated with the molecule design may be retrieved, for example, from the repository associated with the molecule registration system, using the unique molecule identifier of the molecule design. In some cases, the data may be retrieved at any level of the hierarchy including, for example, concept-level, batch-level, component-level, and/or the like. For instance, in some cases, a subset of the data associated with the molecule design may be retrieved including, for example, data associated with the conceptualized version of the molecule design, data associated with one or more specific synthesized batches of the molecule design, data associated with one or more components (e.g., core structure, substructure, and/or the like), and/or the like.

At 660, data associated with each synthesized batch of the molecule design is stored along with a corresponding batch identifier. In some example embodiments, where the molecule design is synthesized, the registration engine of the molecule registration system may register one or more synthesized batches of the molecule design by at least storing data associated with each synthesized batch of the molecule design in the repository associated with the molecule registration system. For example, in some cases, the data associated with a synthesized batch of the molecule design may include one or more parameters associated with the synthesis of the molecule design including, for example, date, time, temperature, equipment specifications, equipment settings, experimental protocols, experimental specifications, experiment-related scientific notes, experimental observations, and/or the like. In some cases, the data associated with the synthesized batch of the molecule design may include one or more measurements associated with the synthesized batch of the molecule design. In some cases, the one or more measurements may include measurements of one or more properties of interest including, for example, drug-like properties such as binding affinity, specificity, biological activity, developability, and/or the like.

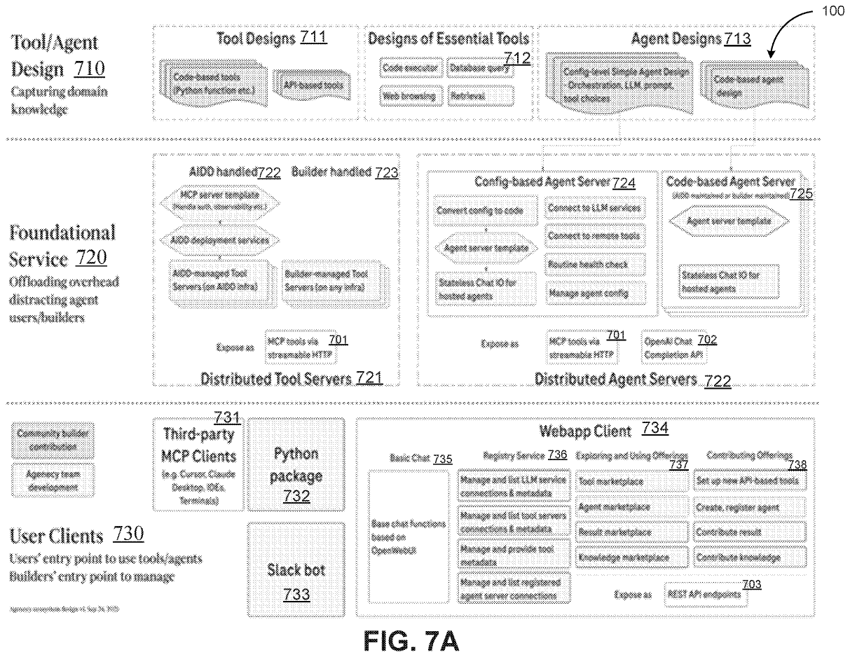

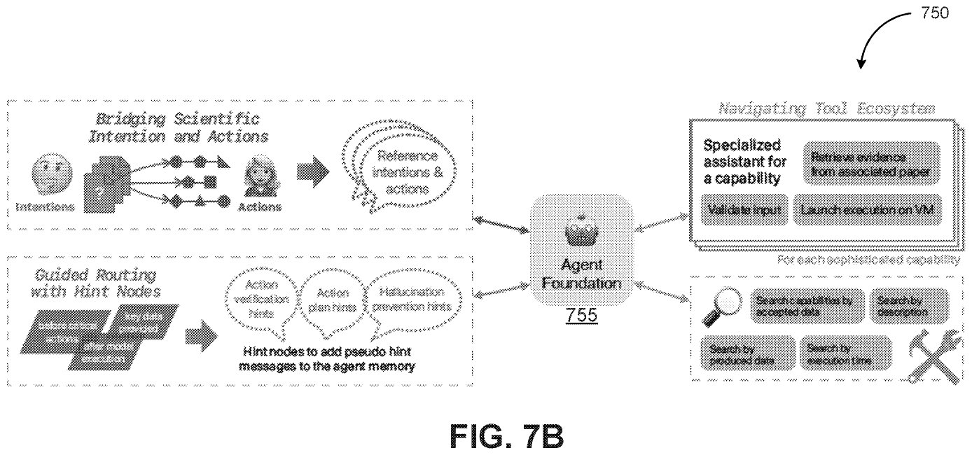

FIG. 7A depicts a schematic diagram illustrating the system design of an example of the drug discovery ecosystem 100, in accordance with some example embodiments. Referring to FIG. 7A, in some example embodiments, the system design of the drug discovery ecosystem 100 may include a tool and agent design layer 710 configured to capture domain knowledge, for example, across different molecular modalities. In some cases, the tool and agent design layer 710 may include drug discovery tool designs 711, such as for code-based tools (e.g., Python functions), application programming interface (API) based tools, and/or the like. As noted, in some cases, examples of drug discovery tools may include computation models for molecule design generation and modification, molecular property prediction, candidate molecule ranking and selection, and/or the like. In some cases, the tool and agent design layer 710 may also include essential tool designs 712, such as for code executor, web browser, database query, retrieval, and/or the like. In some cases, the tool and agent design layer 710 may further include agent designs 713, such as for configuration-level simple agent design (e.g., orchestration, language model, prompt, tool choices, and/or the like), code-based agent design, and/or the like. It should be appreciated that the drug discovery ecosystem 100 may support both configuration level and code-level design of drug discovery tools and artificial intelligence (AI) agents in order to support builders with varying skillsets.

Referring again to FIG. 7A, in some cases, the drug discovery ecosystem 100 may also include a foundational service layer 720. In some cases, the foundational service layer 720 may include one or more distributed tool servers 721, which are exposed to one or more end users of the drug discovery ecosystem 100 as Model Context Protocol (MCP) tools via streamable hypertext transfer protocol (HTTP) 701. In some cases, the distributed tool servers 721 may include those handled by AI-Driven Development (AIDD), which provides server templates (e.g., for handling authentication, observability, and/or the like), deployment servers, and managed tool servers. In some cases, the distributed tool servers 721 may also include builder handled tool servers 723, which are those tool servers managed by the builders of the corresponding drug discovery tools.

In some cases, the foundational service layer 720 may include one or more distributed agent servers 722. In some cases, the one or more distributed agent servers 722 may be exposed to one or more end users of the drug discovery ecosystem 100 as either Model Context Protocol (MCP) tools via streamable hypertext transfer protocol (HTTP) 701 or, alternatively, an application programming interface (API) 702 (e.g., OpenAI Chat Completion API). In some cases, the distributed agent servers 722 may include configuration-based agent server 724, which serves artificial intelligence (AI) agents created based on configurations received from builders interacting with the drug discovery platform 100 (e.g., the controller 110). For example, as shown in FIG. 7A, the configuration based agent server 724 may provide functionalities such as converting configurations (e.g., from a builder) to code, creating agent server templates, and stateless chat input/output (I/O) for hosted agents. In some cases, the configuration based agent server 724 may provide additional functionalities such as connecting to language model (e.g., large language model (LLM)) services, connecting to remote drug discovery tools, performing health checks, and managing agent configurations.

In some cases, the one or more distributed agent servers 722 may also include a code-based agent server 725, which may be either maintained by AI-Driven Development (AIDD) or by the builder of the corresponding artificial intelligence (AI) agent. In some cases, the code-based agent server 725 may serve artificial intelligence (AI) agents created by builders from code. In the example shown in FIG. 7A, the code-based agent server 725 may maintain agent server templates as well as provide stateless chat input/output (I/O) for the artificial intelligence (AI) agents hosted thereon.

Referring again to FIG. 7A, in some cases, the drug discovery ecosystem 100 may include a user client layer 730, which serves as an entry point for builders to manage the available drug discovery tools and artificial intelligence (AI) agents as well as an entry point for end users consuming the same drug discovery tools and artificial intelligence (AI) agents. As shown in FIG. 7A, the user client layer 730 may include one or more third party Model Context Protocol (MCP) clients 731, a Python package, and a chatbot 733. In some cases, the user client layer 730 may further include a webapp client 734 exposed to one or more builders and end users as application programming interface (API) endpoints 703 (e.g., Representational State Transfer (REST) API endpoints). In some cases, the webapp client 734 may provide a chat function 735 (e.g., basic chat functions based on OpenWebUI). In some cases, the webapp client 734 may also include a registry service 736 for managing and listing language model (e.g., large language model (LLM)) service connections and metadata, tool server connections and metadata, drug discovery tool metadata, and registered agent server connections. In some cases, the webapp client 734 may also enable the exploration and utilization of various offerings 737, such as drug discovery tools and artificial intelligence (AI) agents, by one or more end users interacting with the drug discovery ecosystem 100. In the example shown in FIG. 7A, the webapp client 734 may provide a drug discovery tool marketplace (e.g., the drug discovery tool marketplace 400 shown in FIG. 4A), an artificial intelligence (AI) agent marketplace (e.g., the artificial intelligence (AI) agent marketplace 450 shown in FIG. 4B), a results marketplace (e.g., for results from the application of various drug discovery tools, the execution of drug discovery workflows, and/or the like), and a knowledge marketplace (e.g., the internal data 252 and external data 254 shown in FIG. 2B). In some cases, the webapp client 734 may further enable the contribution of offerings 738, such as drug discovery tools and artificial intelligence (AI) agents, for example, by one or more builders interacting with the drug discovery ecosystem 100. In the example shown in FIG. 7A, the webapp client 734 may set up new application programming interface (API) based drug discovery tools, create and register artificial intelligence (AI) agents, contribute results (e.g., from the application of various drug discovery tools, the execution of drug discovery workflows, and/or the like), and contribute knowledge (e.g., the internal data 252 and external data 254 shown in FIG. 2B).

FIG. 7B depicts a schematic diagram illustrating an example of an artificial intelligence (AI) agent design 750 that utilizes a synthesized intention-action pool to inform an artificial intelligence (AI) agent of possible trajectories mapping high-level intensions with concrete operations in a drug discovery workflow, in accordance with some example embodiments. Referring to FIG. 7B, in some cases, the agent design 750 may leverage index-inspired search tools as well as specialized artificial intelligence agents to orchestrate the execution of individual drug discovery tools in a drug discovery pipeline while high-level action planning is performed by a primary artificial intelligence (AI) agent. In some cases, one or more hint nodes may be added to memory provide timely reminders during agent routing between the primary artificial intelligence (AI) agent and one or more specialized artificial intelligence (AI) agents.

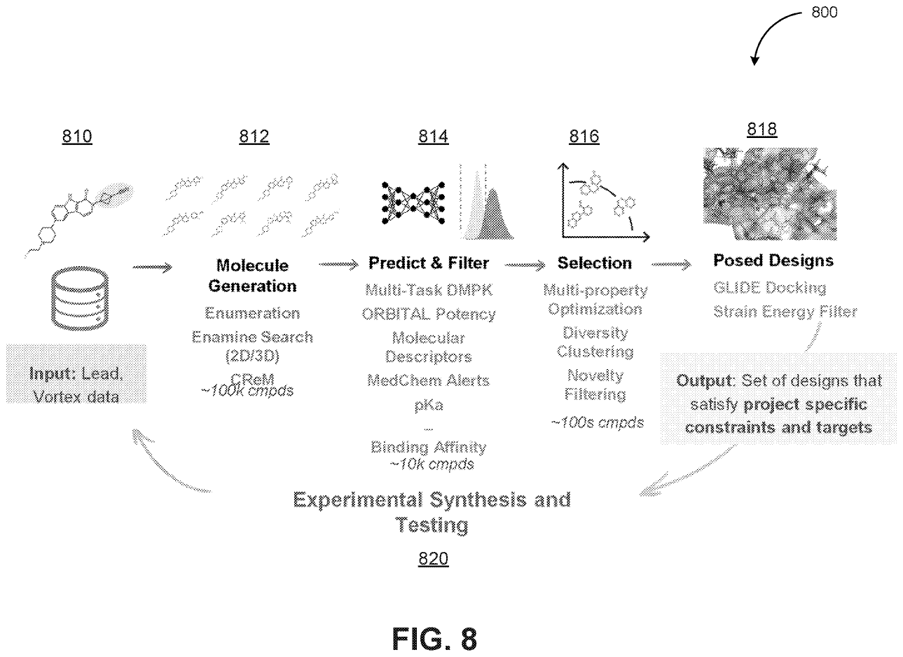

FIG. 8 depicts a schematic diagram illustrating an example of a drug discovery workflow 800 orchestrated by an artificial intelligence (AI) agent, in accordance with some example embodiments. Referring to FIG. 8, in some cases, the drug discovery workflow 800 may be orchestrated by an artificial intelligence (AI) agent (e.g., the one or more agents 120) configured by a builder interacting with a controller of a drug discovery ecosystem (e.g., the controller 110 of the drug discovery ecosystem 100 in FIG. 1). In some cases, the drug discovery workflow 800 may include receiving one or more inputs (e.g., a lead molecule) at 810. In some cases, where the one or more inputs include a lead molecule (e.g., a small molecule drug compound, a therapeutic protein or peptide, a nucleic acid, and/or the like), the one or more inputs may include a unique molecule identifier of the lead molecule such that data associated with the lead molecule may be retrieved from a molecule registration system (e.g., the molecule registration system 140 in FIG. 1) before continuing the drug discovery workflow 800.

Referring again to FIG. 8, in some cases, one or more molecule designs may be generated based on the lead molecule at 812. In some cases, the artificial intelligence (AI) agent may orchestrate the drug discovery workflow 800 by at least applying a drug discovery tool (e.g., a molecule design computation model) to generate the one or more molecule designs. In some cases, each molecule design may be registered with the molecule registration system (e.g., the molecule registration system 140 in FIG. 1) and assigned a corresponding unique molecule identifier for further use in the drug discovery workflow 800. In some cases, at 814, the artificial intelligence (AI) agent may apply, to the one or more molecule designs, one or more drug discovery tools (e.g., one or more property computation model, a multi-property computation model, and/or the like) to predict one or more properties of interest present in each molecule design. In some cases, at 816, the artificial intelligence (AI) agent may continue orchestrating the drug discovery workflow 800 by applying a drug discovery tool (e.g., a selection computation model) to identify molecule designs for further development based on individual or multiple properties of interest including, in some cases, the diversity and novelty of each molecule design. In some cases, at 818, the artificial intelligence (AI) agent may apply a drug discovery tool (e.g., a structure computation model) to predict the structure of each molecule design in complex with one or more other molecules (e.g., protein-ligand complex, immune complex, and/or the like). As shown in FIG. 8, in some case, a subset of molecule designs satisfying certain project specific constraints and targets may be selected for experimental synthesis and testing at 820. In some cases, the experimental synthesis and testing may yield additional lead molecules for one or more subsequent iterations of the drug discovery workflow 800, which may be executed to further optimize those molecule designs that have undergone experimental synthesis and testing to validate one or more properties of interest. In some cases, the drug discovery workflow 800 may be iterated until one or more criteria are satisfied. For example, in some cases, the drug discovery workflow 800 may be iterated until a threshold quantity of iterations are performed. Alternatively and/or additionally, the drug discovery workflow 800 may be iterated until a threshold quantity of molecule designs exhibiting one or more properties of interest are generated.

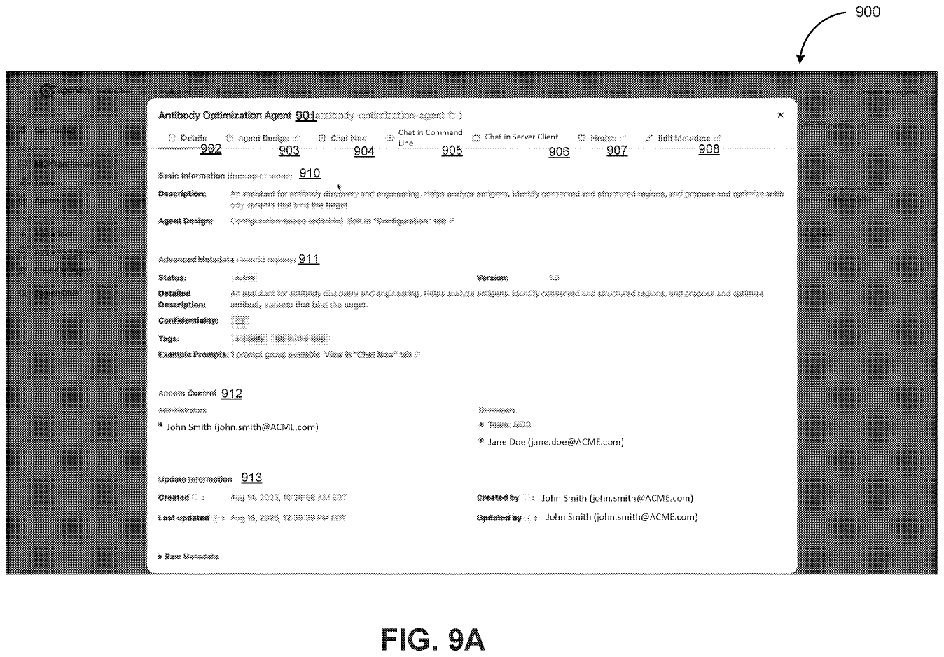

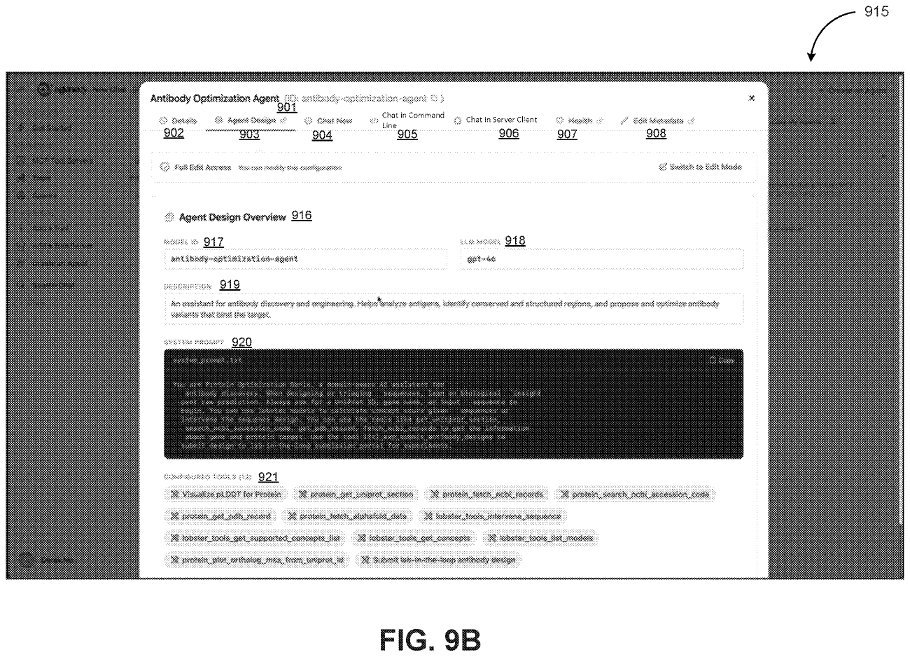

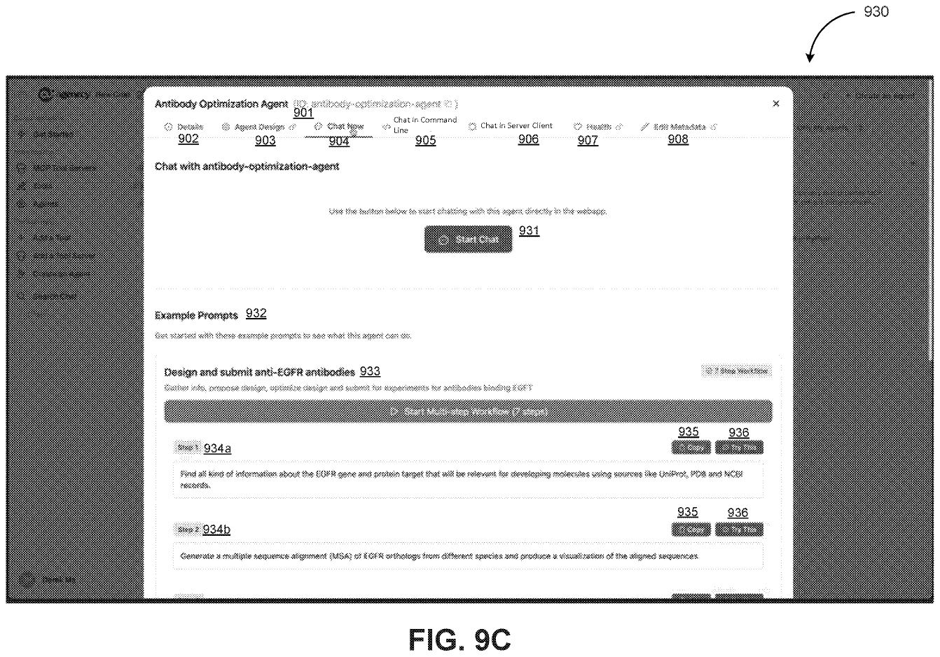

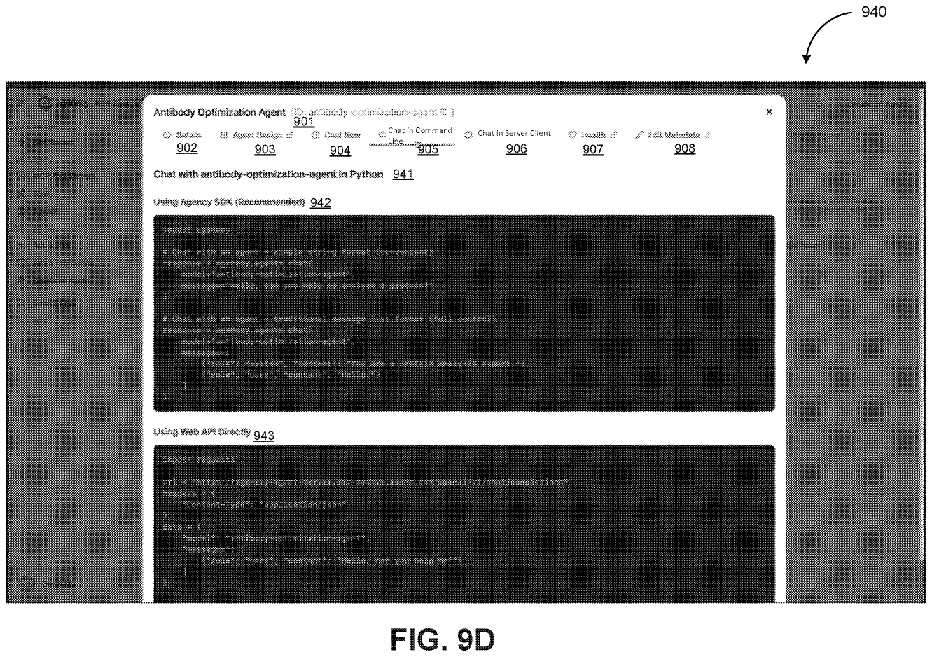

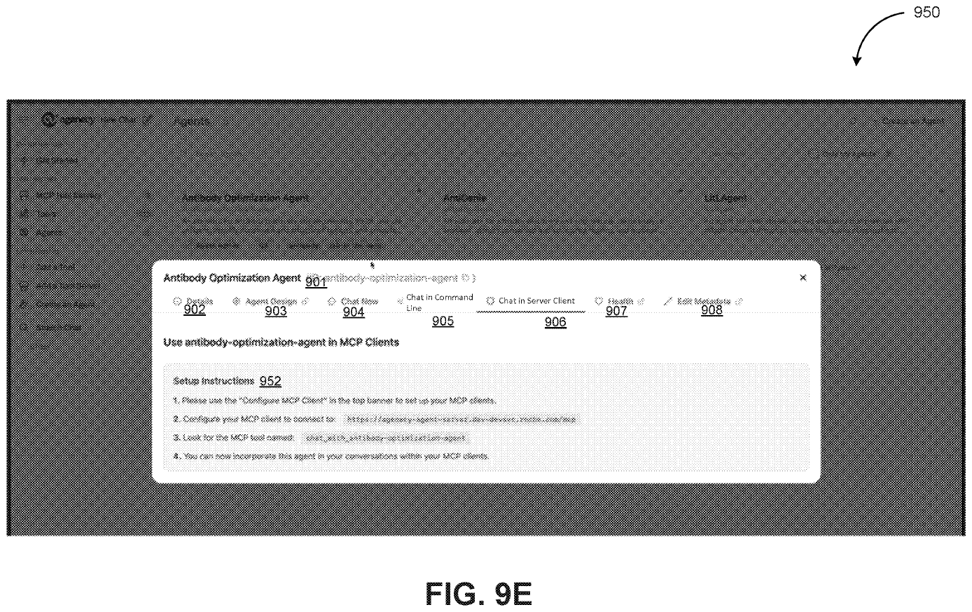

FIG. 9A depicts a screenshot of an example of a user interface (UI) 900 for interacting with an artificial intelligence (AI) agent for orchestrating a drug discovery workflow, in accordance with some example embodiments. Referring to FIG. 9A, in some cases, the user interface (UI) 900 may support interactions between, for example, one or more builders (e.g., the builder 305 in FIG. 3A) and/or end users (e.g., the end user 310 in FIG. 3B), and an antibody optimization agent 901. In some cases, the user interface (UI) 900 may include multiple tabs, each of which providing a different functionality. In the example shown in FIG. 9A, the tabs may include a details tab 902, an agent design tab 903, a chat now tab 904, a chat in command line tab 905, a chat in sever client tab 906, a health tab 907, and an edit metadata tab 908.

Referring again to FIG. 9A, the user interface (UI) 900 may be generated, for example, by the controller 110, for display at the client device 160, when the details tab 902 is selected. In some cases, the details tab 902 may include basic information 910 associated with the antibody optimization agent 901. In some cases, the basic information 910 may include a description of the antibody optimization agent 901 (e.g., “An assistant for antibody discovery and engineering. Helps analyze antigens, identify conserved and structured regions, and propose and optimize antibody variants that bind the target.”). In some cases, the basic information 910 may further include an indication of whether the design of the antibody optimization agent 901 is code-based or configuration-based. In some cases, where the antibody optimization agent 901 is associated with a code-based design, the behaviors of the antibody optimization agent 901 may be defined programmatically. By contrast, where the antibody optimization agent 901 is associated with a configuration-based design, the behaviors of the antibody optimization agent 901 may be defined in a declarative configuration file (e.g., an .agent.md file with frontmatter in a data serialization language such as Yet Another Markup Language (YAML)).

In some cases, the details tab 902 of the antibody optimization agent 901 may also include advanced metadata 911 associated with the antibody optimization agent 901. In some cases, the advanced metadata 911 may include the status (e.g., active, inactive) and a detailed description of the antibody optimization agent 901. In some cases, the advanced metadata 911 may also include a confidentiality of the antibody optimization agent 901 (which may determine its permissions and accessibility), one or more tags to facilitate the exploration and utilization of the antibody optimization agent 901, and example prompts associated with the antibody optimization agent 901. In the example shown in FIG. 9A, these example prompts may be visible for viewing under a different tab (e.g., the chat now tab 904).

In some cases, the details tab 902 of the antibody optimization agent 901 may also include access control 912 data. For example, in FIG. 9A, the access control 912 data may include the administrators and developers associated with the antibody optimization agent 901. In some cases, the details tab 902 of the antibody optimization agent 901 may further include update information 913. Examples of the update information 913 may include the date and time of creation, the identity of the builder who created the antibody optimization agent 901, the date and time of the last update, the identity of the builder who last updated the antibody optimization agent 901, and/or the like.

FIG. 9B depicts a screenshot of another example of a user interface (UI) 915 for interacting with an artificial intelligence (AI) agent for orchestrating a drug discovery workflow, in accordance with some example embodiments. Referring to FIG. 9B, in some cases, the user interface (UI) 915 may be generated for example, by the controller 110, for display at the client device 160, upon selection of the agent design tab 903. In some cases, the agent design tab 903 may include an agent design overview 916. In some cases, the agent design overview 916 may include a model identifier field 917 in which one or more builders of the antibody optimization agent 901 may input (or edit) an identifier (e.g., antibody-optimization-agent). In some cases, the identifier of the antibody optimization agent 901 may be displayed in each of user interfaces associated with the antibody optimization agent 901. In some cases, the agent design overview 916 may also include a large language model (LLM) field 918 in which one or more builders of the antibody optimization agent 901 may input (or edit) one or more language models (e.g., large language models (LLM)) coupled with the antibody optimization agent 901 to support natural language based interactions therewith. In some cases, the agent design overview 916 may include a system prompt field 920 in which one or more builders of the antibody optimization agent 901 may input (or edit) the system prompt (e.g., a system_prompt.txt file) associated with the antibody optimization agent 901. In some cases, the system prompt associated with the antibody optimization agent 901 may include a set of instructions defining one or more of the personality, role, rules, and interactive behaviors of the antibody optimization agent 901 (e.g., “lean on biological insight over raw prediction,” “always ask for a UniProt ID, gene name, or input sequence to begin,” “you can use models to calculate concept score given sequences or interview the sequence design,” and/or the like). In some cases, the system prompt of the antibody optimization agent 901 may guide its responses to align with specific goals, tones (e.g., professional, friendly), and ethical boundaries. In some cases, the system prompt of the antibody optimization agent 901 may provide context, set guardrails (e.g., prohibited behaviors), and specify tasks, thus ensuring the consistency and relevance of its output.

Referring again to FIG. 9B, in some cases, the agent design overview 916 of the antibody optimization agent 901 may also include a selection of configured tools 921 bound to the antibody optimization agent 901. In some cases, the selection of configured tools 921 may include one or more drug discovery tools selected by one or more builders of the antibody optimization agent 901 from those available from the drug discovery ecosystem 100 (e.g., the drug discovery tool marketplace 400 shown in FIG. 4A). In some cases, the antibody optimization agent 901 may apply one or more drug discovery tools from the selection of configured tools 921 as part of orchestrating a drug discovery workflow, for example, to optimize an antibody. For example, in some cases, the antibody optimization agent 901 may be configured to orchestrate a predetermined drug discovery workflow, in which case the drug discovery tools in the selection of configured tools 921 may be applied in a fixed sequence. Alternatively, in some cases, the antibody optimization agent 901 may be configured to orchestrate an adaptive drug discovery workflow, in which case the drug discovery tools in the selection of configured tools 921 may be applied to a dynamic sequence determined by the antibody optimization agent 901.

FIG. 9C depicts a screenshot of another example of a user interface (UI) 930 for interacting with an artificial intelligence (AI) agent for orchestrating a drug discovery workflow, in accordance with some example embodiments. Referring to FIG. 9C, in some cases, the user interface (UI) 930 may be generated for example, by the controller 110, for display at the client device 160, upon selection of the chat now tab 904. In some cases, the user interface (UI) 930 may provide the option to chat with the antibody optimization agent 901 including initiating a chat with a start chat button 931. In some cases, the user interface (UI) 930 may provide example prompts 932 that may be entered into the chat with the antibody optimization agent 901 to launch one or more drug discovery workflows for orchestration by the antibody optimization agent 901. In some cases, the example prompts 932 may be organized into different drug discovery workflows associated with the antibody optimization agent 901. For example, FIG. 9D shows a drug discovery workflow to design and submit anti-EGFR antibodies 933, which is a drug discovery workflow having seven constituent operations. In some cases, the example prompts 932 may include, for each operation in the design and submit anti-EGFR antibodies 933 drug discovery workflow, one or more sample prompts. For instance, for a first operation 934a of the design and submit anti-EGFR antibodies 933 drug discovery workflow, the sample prompt may include “Find all kind of information about the EGFR gene and protein target that will be relevant for developing molecules using sources like UniProt, PDB, and NCIBI records.” In some cases, the user interface (UI) 930 may include, for the sample prompt associated with the first operation 934a of the design and submit anti-EGFR antibodies 933 drug discovery workflow, a button 935 to copy (and paste) the sample prompt into the chat with the antibody optimization agent 901 and a button 936 to input the sample prompt directly. Similarly, in some cases, for a second operation 934b of the design and submit anti-EGFR antibodies 933 drug discovery workflow, the sample prompt may include “Generate a multiple sequence alignment (MSA) of EGFR orthologs from different species and produce a visualization of the aligned sequences.” In some cases, the user interface (UI) 930 may include, for the sample prompt associated with the second operation 934b in the design and submit anti-EGFR antibodies 933 drug discovery workflow, the button 935 to copy (and paste) the sample prompt into the chat with the antibody optimization agent 901 and the button 936 to input the sample prompt directly.

FIG. 9D depicts a screenshot of another example of a user interface (UI) 940 for interacting with an artificial intelligence (AI) agent for orchestrating a drug discovery workflow, in accordance with some example embodiments. Referring to FIG. 9D, in some cases, the user interface (UI) 940 may be generated for example, by the controller 110, for display at the client device 160, upon selection of the chat in command line tab 905. In some cases, the controller 110 of the drug discovery ecosystem 100 may support various forms of chats for interacting with the antibody optimization agent 901. For example, in addition to the chat now option provided under the chat now tab 904 shown in FIG. 9C, the controller 110 of the drug discovery ecosystem 100 may also support chats that are conducted in a command line interface under the chat in command line tab 905. In some cases, command line chats may be conducted using the software development kit (SDK) 942 or, alternatively, using the web application programming interface (API) directly 943.

FIG. 9E depicts a screenshot of another example of a user interface (UI) 950 for interacting with an artificial intelligence (AI) agent for orchestrating a drug discovery workflow, in accordance with some example embodiments. Referring to FIG. 9E, in some cases, the user interface (UI) 950 may be generated for example, by the controller 110, for display at the client device 160, upon selection of the chat in server client tab 906. As noted, in some cases, the controller 110 of the drug discovery ecosystem 100 may support various forms of chats for interacting with the antibody optimization agent 901. For example, in addition to the chat now option provided under the chat now tab 904 shown in FIG. 9C and the command line operation provide under the chat in command line tab 905 shown in FIG. 9D, the controller 110 of the drug discovery ecosystem 100 may also support chats that are conducted in a server client (e.g., a Model Context Protocol (MCP) server client) under the chat in server client tab 906. As shown in FIG. 9E, in some cases, the controller 110 may support the use of the antibody optimization agent 901 in one or more server clients. For example, in some cases, the antibody optimization agent 901 may be used in one or more Model Context Protocol (MCP) server clients. In some cases, a Model Context Protocol (MCP) server may be a type of drug discovery tool server operating in accordance with the Model Context Protocol (MCP) standards for connecting artificial intelligence (AI) applications to external systems. In some cases, the chat in server client tab 906 may include setup instructions 952 for configuring the server client (e.g., Model Context Protocol (MCP) client) to connect to the tool server (e.g., Model Context Protocol (MCP) server) hosting the antibody optimization agent 901 and locating the chat tool associated with the antibody optimization agent 901 thereon.

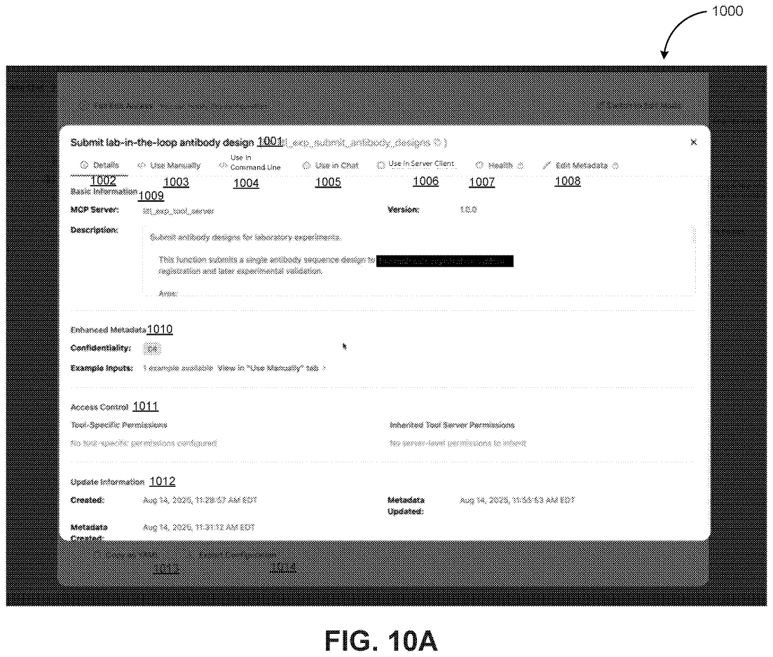

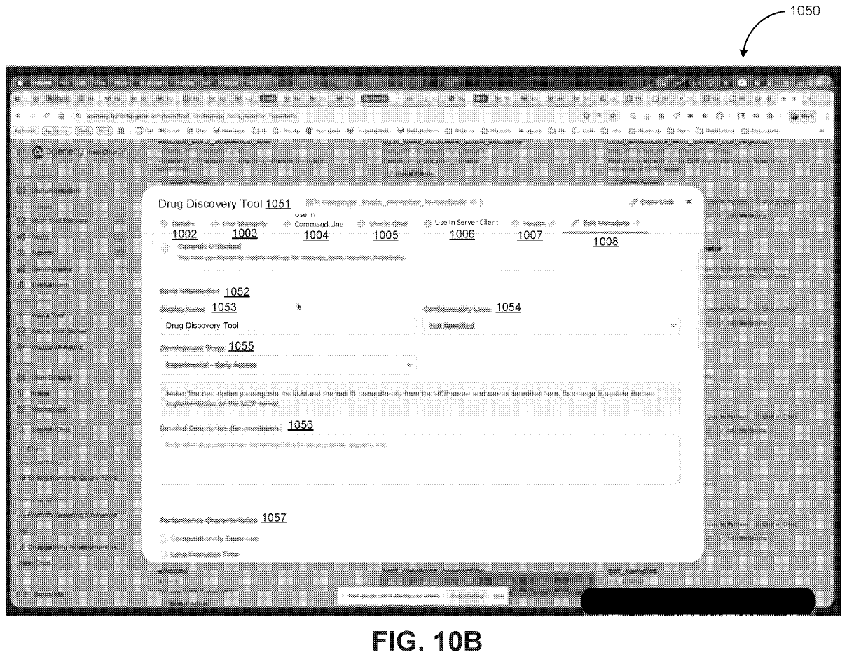

FIG. 10A depicts a screenshot of an example of a user interface (UI) 1000 for interacting with a drug discovery tool, in accordance with some example embodiments. Referring to FIG. 10A, in some cases, the user interface (UI) 1000 may support interactions between, for example, one or more end users (e.g., the end user 310 in FIG. 3B) and the submit lab-in-the-loop antibody design 1001 drug discovery tool. In some cases, the user interface (UI) 1000 may include multiple tabs, each of which provides a different functionality. In the example shown in FIG. 10A, the tabs may include a details tab 1002, a use manually tab 903, a use in command line tab 1004, a use in chat tab 1005, a user in server client tab 1006, a health tab 1007, and an edit metadata tab 1008. In some cases, the user interface (UI) 1000 may be generated for example, by the controller 110, for display at the client device 160, upon selection of the details tab 1002.

Referring again to FIG. 10A, in some cases, the details tab 1002 of the submit lab-in-the-loop antibody design 1001 drug discovery tool may include basic information 1009 associated with the lab-in-the-loop antibody design 1001 drug discovery tool. For example, in FIG. 10A, the basic information 1009 may include an identity of the tool server (e.g., Model Context Protocol (MCP) server) hosting the submit lab-in-the-loop antibody design 1001 drug discovery tool (e.g., litl_exp_tool_server) and a version of the submit lab-in-the-loop antibody design 1001 drug discovery tool (e.g., 1.0.0). In some cases, the basic information 1009 may further include a description of the submit lab-in-the-loop antibody design 1001 drug discovery tool (e.g., “This function submits a single antibody sequence design to the molecule registration system for registration and later experimental validation.”).

In some cases, the details tab 1002 of the submit lab-in-the-loop antibody design 1001 drug discovery tool may include enhanced metadata 1010. In some cases, the enhanced metadata 1010 of the submit lab-in-the-loop antibody design 1001 drug discovery tool may include the confidentiality of the submit lab-in-the-loop antibody design 1001 drug discovery tool (which may determine its permissions and accessibility) and example inputs. In the example shown in FIG. 10A, these example inputs may be visible for viewing under a different tab (e.g., the use manually tab 1003).

In some cases, the details tab 1002 of the submit lab-in-the-loop antibody design 1001 drug discovery tool may include access controls 1011. In some cases, the access control 1011 may include tool-specific permissions, for which none are currently configured for the submit lab-in-the-loop antibody design 1001 drug discovery tool in the example shown in FIG. 10A. In some cases, the access control 1011 may also include inherited tool server permissions, which are associated with the tool server (e.g., Model Control Protocol (MCP) server) hosting the submit lab-in-the-loop antibody design 1001 drug discovery tool. In the example shown in FIG. 10A, the access control 1011 of the submit lab-in-the-loop antibody design 1001 drug discovery tool shows no inherited server level permissions. In some cases, the details tab 1002 of the submit lab-in-the-loop antibody design 1001 drug discovery tool may also include update information 1012. In the example shown in FIG. 10A, the update information 1012 may include the dates and times of tool creation, metadata creation, and metadata update. In some cases, the details tab 1002 of the submit lab-in-the-loop antibody design 1001 drug discovery tool may include a copy as YAML button 1013 providing the option of replicating the details of the submit lab-in-the-loop antibody design 1001 drug discovery tool into a data serialization language (e.g., Yet Another Markup Language (YAML)) file. In some cases, the details tab 1002 of the submit lab-in-the-loop antibody design 1001 drug discovery tool may include a copy as YAML button 1013 providing the option of exporting the configurations of the submit lab-in-the-loop antibody design 1001 drug discovery tool.

FIG. 10B depicts a screenshot of an example of a user interface (UI) 1050 for configuring a drug discovery tool, in accordance with some example embodiments. Referring to FIG. 10A, in some cases, the user interface (UI) 1050 may support interactions between, for example, one or more end users (e.g., the end user 310 in FIG. 3B) and a drug discovery tool 1051. In some cases, the user interface (UI) 1050 may include multiple tabs, each of which providing a different functionality. Similar to the example of the user interface (UI) 1000 shown in FIG. 10A, in the example shown in FIG. 10B, the tabs may include the details tab 1002, the use manually tab 903, the use in command line tab 1004, the use in chat tab 1005, the user in server client tab 1006, the health tab 1007, and the edit metadata tab 1008. In some cases, the user interface (UI) 1050 may be generated for example, by the controller 110, for display at the client device 160, upon selection of the edit metadata tab 1008.

Referring again to FIG. 10B, the edit metadata tab 1008 of the drug discovery tool 1051 may include basic information 1052. In some cases, the basic information 1052 of the drug discovery tool 1051 may include a display name 1053 of the drug discovery tool 1051 (e.g., “Drug Discovery Tool”). In some cases, the basic information 1052 of the drug discovery tool 1051 may also include a confidentiality level 1054 (e.g., “Not Specified”) and a development stage 1055 (e.g., “Experimental-Early Access”) of the drug discovery tool 1051. In some cases, the basic information 1052 of the drug discovery tool 1051 may also include a detailed description 1056 of the drug discovery tool 1051, which may include extended documentation associated with the drug discovery tool 1051 such as source code, papers, and/or the like. In some cases, the basic information 1052 of the drug discovery tool 1051 may include the performance characteristics 1057 of the drug discovery tool 1051. For example, in FIG. 10B, the performance characteristics 1057 of the drug discovery tool 1051 may include selectable options for “Computationally Expensive” and “Long Execution Time.” It should be appreciated that the display name 1052, the confidentiality level 1054, the development stage 1055, the detailed description 1056, and the performance characteristics 1057 of the drug discovery tool 1051 may be associated with editable fields for the input (or editing) of the corresponding information.

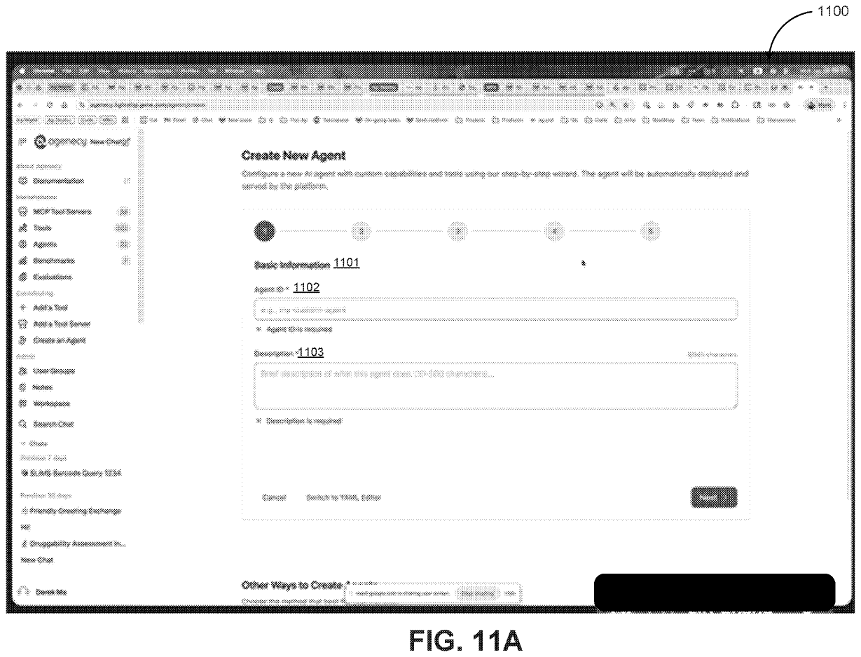

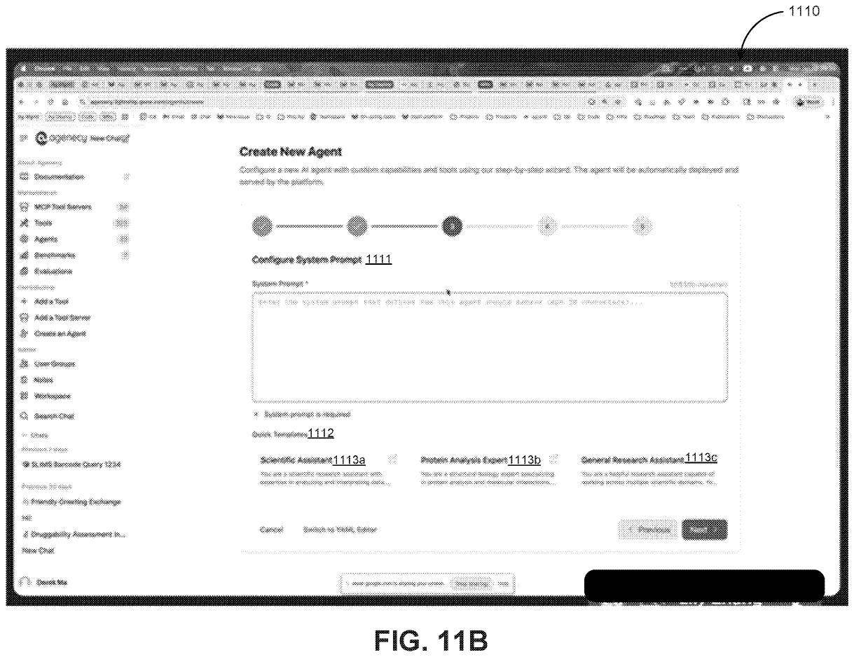

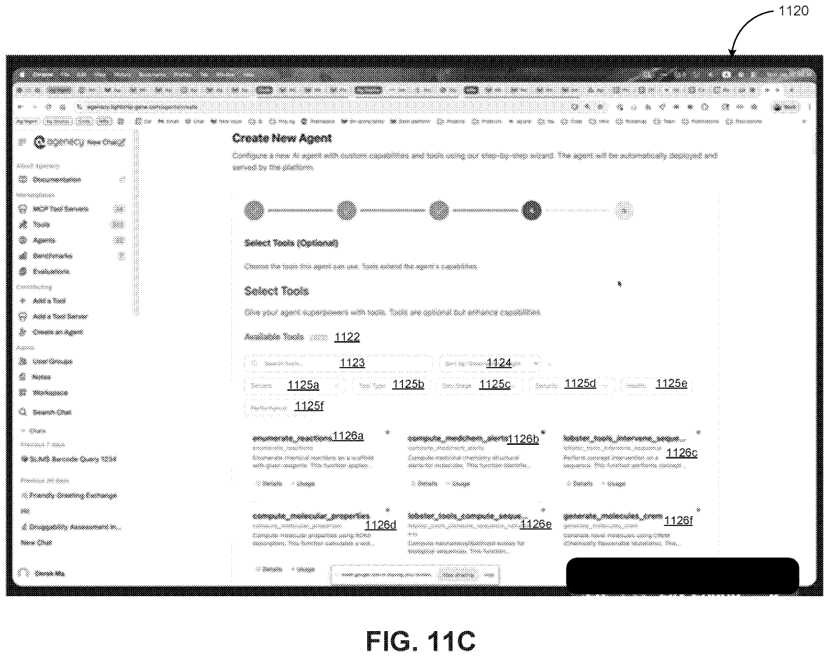

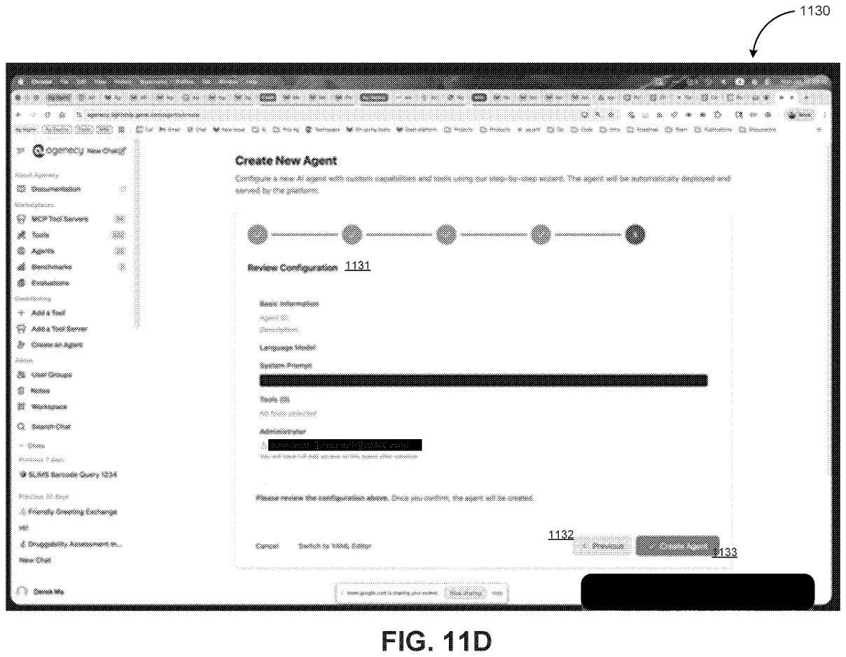

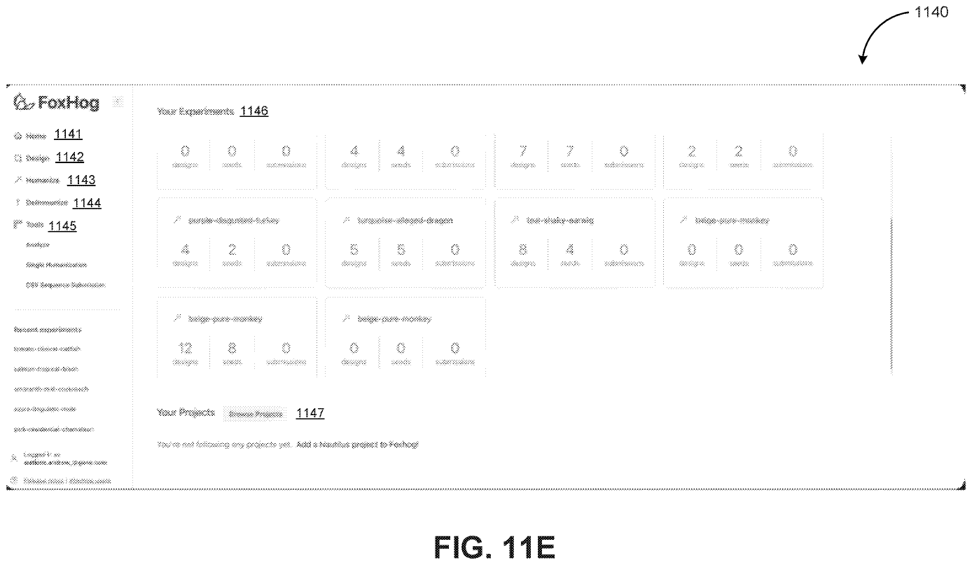

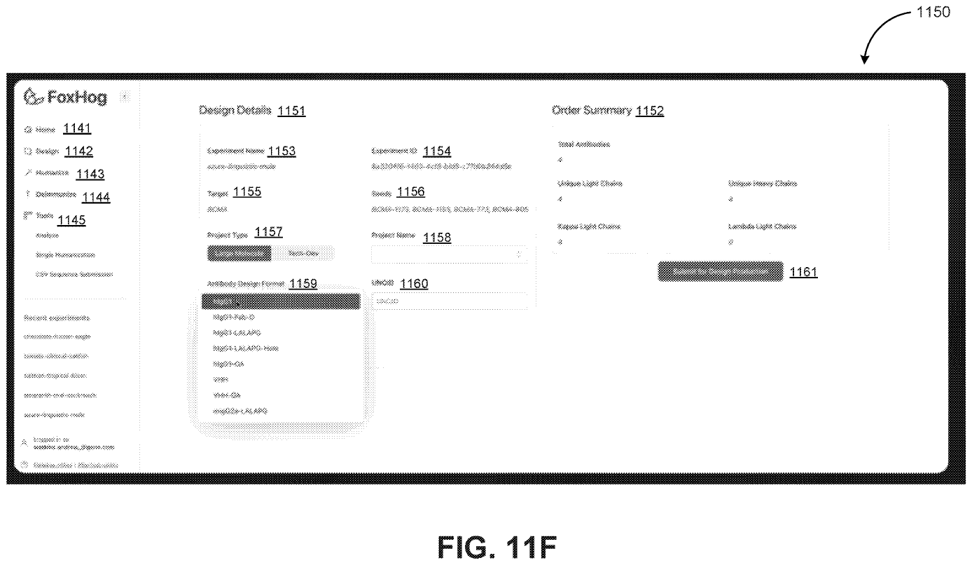

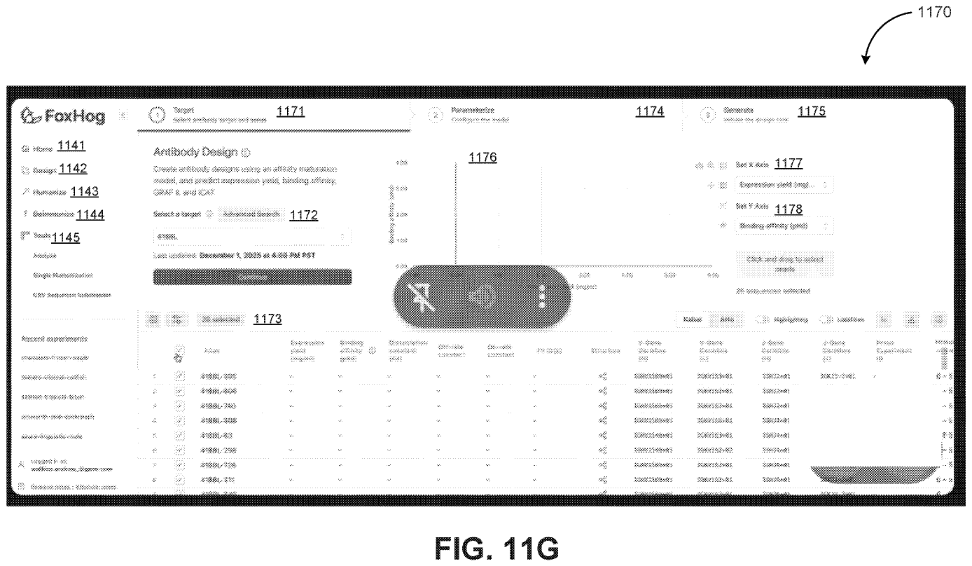

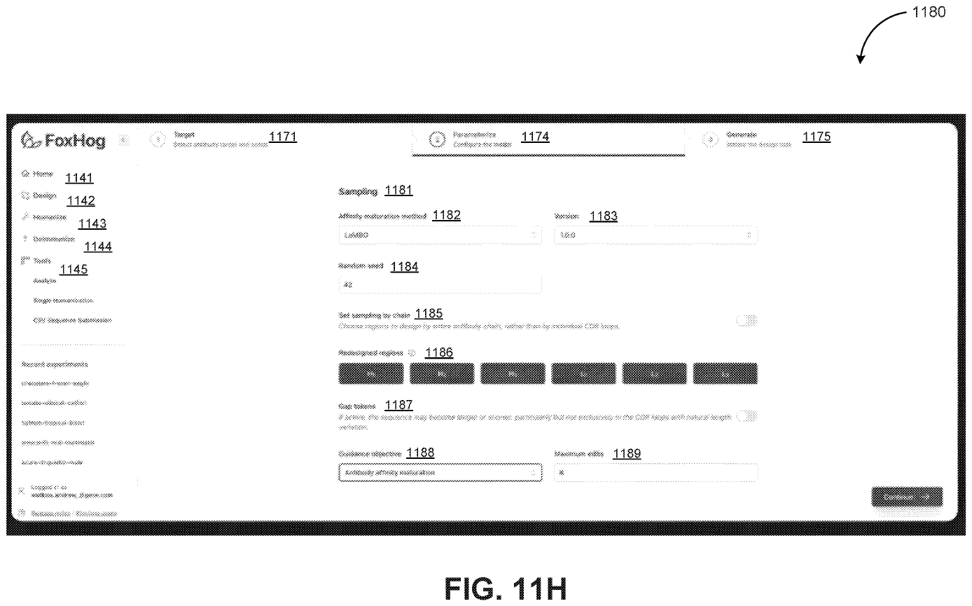

FIG. 11A depicts a screenshot of an example of a user interface (UI) 1100 for configuring an artificial intelligence (AI) agent for orchestrating a drug discovery workflow, in accordance with some example embodiments. Referring to FIG. 11A, in some cases, the user interface (UI) 1100 may support interactions between, for example, one or more builders (e.g., the builder 305 in FIG. 3A) and the controller 110 to create and configure a new artificial intelligence (AI) agent to orchestrate one or more drug discovery workflows. As described in more detail below, the user interface (UI) 1100 may be a part of a sequence of user interfaces (UIs) in a workflow to configure a new artificial intelligence (AI) agent with custom capabilities and drug discovery tools for automatic service and deployment within the drug discovery ecosystem 100.

Referring again to FIG. 11A, in some cases, the user interface (UI) 1100 may include one or more editable fields for a builder to input (or edit) basic information 1101 associated with the new artificial intelligence (AI) agent. For example, in FIG. 11A, the user interface (UI) 1100 may include an agent ID field 1102 for inputting (or editing) an identifier of the artificial intelligence (AI) agent. Furthermore, in FIG. 11A, the user interface (UI) 1100 may include a description field 1103 for inputting (or editing) a brief description of the new artificial intelligence (AI) agent and its functionalities. In the example shown in FIG. 11A, inputs may be required for both the agent ID field 1102 and the description field 1103.

FIG. 11B depicts a screenshot of another example of a user interface (UI) 1110 for configuring an artificial intelligence (AI) agent for orchestrating a drug discovery workflow, in accordance with some example embodiments. Referring to FIG. 111B, in some cases, the user interface (UI) 1110 may support interactions between, for example, one or more builders (e.g., the builder 305 in FIG. 3A) and the controller 110 to create and configure a new artificial intelligence (AI) agent to orchestrate one or more drug discovery workflows. In some cases, the user interface (UI) 1110 may be a part of a sequence of user interfaces (UIs) in a workflow to configure a new artificial intelligence (AI) agent with custom capabilities and drug discovery tools for automatic service and deployment within the drug discovery ecosystem 100.

Referring again to FIG. 11B, in some cases, the user interface (UI) 1110 may include one or more editable fields for a builder to input (or edit) the system prompt 1111 associated with the new artificial intelligence (AI) agent. In some cases, the system prompt 1111 of the artificial intelligence (AI) agent may define the agent's behavior. For example, in some cases, the system prompt 111 may include a set of instructions defining one or more of the personality, role, rules, and interactive behaviors of the artificial intelligence (AI) agent. In some cases, the system prompt 111 of the artificial intelligence (AI) agent may guide its responses to align with specific goals, tones (e.g., professional, friendly), and ethical boundaries. In some cases, the system prompt 1111 of the artificial intelligence (AI) agent may provide context, set guardrails (e.g., prohibited behaviors), and specify tasks, thus ensuring the consistency and relevance of its output. In the example shown in FIG. 111B, the system prompt 1111 may be a required input for the configuration of the new artificial intelligence (AI) agent.

In some cases, the user interface (UI) 1110 may display a selection of quick templates 1112 for configuring the new artificial intelligence (AI) agent. For example, in FIG. 11n, the quick templates 1112 may include definition templates for a scientific assistant 1113a, a protein analysis expert 1113b, and a general research assistant 1113c. In some cases, each quick template 1112 may include one or more editable fields for receiving one or more inputs configuring a corresponding artificial intelligence (AI) agent. For instance, in some cases, the quick templates 1112 may provide a template system prompt for input into the field for system prompt 1111 for use as is or for further editing.

FIG. 11C depicts a screenshot of another example of a user interface (UI) 1120 for configuring an artificial intelligence (AI) agent for orchestrating a drug discovery workflow, in accordance with some example embodiments. Referring to FIG. 11C, in some cases, the user interface (UI) 1120 may support interactions between, for example, one or more builders (e.g., the builder 305 in FIG. 3A) and the controller 110 to create and configure a new artificial intelligence (AI) agent to orchestrate one or more drug discovery workflows. In some cases, the user interface (UI) 1120 may be a part of a sequence of user interfaces (UIs) in a workflow to configure a new artificial intelligence (AI) agent with custom capabilities and drug discovery tools for automatic service and deployment within the drug discovery ecosystem 100.

Referring again to FIG. 11C, in some cases, the user interface (UI) 1120 may enable a builder to further configure the new artificial intelligence (AI) agent by at least selecting one or more drug discovery tools for inclusion in the drug discovery workflow orchestrated by the artificial intelligence (AI) agent. As noted, in some cases, the drug discovery workflow may be a predetermined drug discovery workflow that the artificial intelligence (AI) agent orchestrates by invoking the selected drug discovery tools in a preset sequence. Alternatively, the drug discovery workflow may be an adaptive drug discovery workflow in which the artificial intelligence (AI) agent dynamically selects the drug discovery tools that are applied throughout, for example, based on its interactions with one or more end users and the inputs received therefrom. In some cases, the user interface (UI) 1120 may support the selection of drug discovery tools by at least displaying a gallery (or menu) of available tools 1122. For example, in FIG. 11C, the available tools 1122 include enumerate reactions 1126a, compute medchem alerts 1126b, lobster_tools_intervene_sequence 1126c, compute_molecular_properties 1126d, lobster_tools_compute_sequence 1126e, and generate_molecules_CReM 1126f. In some cases, the user interface (UI) 1120 may also provide a search field 1123 for inputting a text-based search of the available tools 1122. In some cases, the user interface (UI) 1120 may further include a sort function 1124 to sort the available tools 1122 based on one or more criteria (e.g., description length). In some cases, the user interface (UI) 1120 may also include one or more filtering functions 1125 including, for example, filtering by tool server 1125a, tool type 1125b, development stage 1125c, security (or confidentiality) 1125d, health 1125e, and performance characteristics 1125f. It should be appreciated that the available tools 1122 may also be searched, sorted, and/or filtered based on performance benchmarks, including those determined based on user feedback (e.g., passive feedback, active feedback, and/or the like).

FIG. 11D depicts a screenshot of another example of a user interface (UI) 1130 for configuring an artificial intelligence (AI) agent for orchestrating a drug discovery workflow, in accordance with some example embodiments. Referring to FIG. 11D, in some cases, the user interface (UI) 1130 may support interactions between, for example, one or more builders (e.g., the builder 305 in FIG. 3A) and the controller 110 to create and configure a new artificial intelligence (AI) agent to orchestrate one or more drug discovery workflows. In some cases, the user interface (UI) 1130 may be a part of a sequence of user interfaces (UIs) in a workflow to configure a new artificial intelligence (AI) agent with custom capabilities and drug discovery tools for automatic service and deployment within the drug discovery ecosystem 100. As shown in FIG. 11D, in some cases, the user interface (UI) 1130 may provide a summary 1131 of the configurations of the artificial intelligence (AI) agent for review. In the example shown in FIG. 11D, the configuration summary 1131 may include basic information (e.g., agent identifier, description), associated language model, system prompt, associated drug discovery tools, and administrator. In some cases, the user interface (UI) 1130 may provide an option to return to a previous configuration page with the previous button 1132 as well as an option to create the agent based on the configuration summary 1131 with the create agent button 1133.

FIG. 11E depicts a screenshot of another example of a user interface (UI) 1140 for configuring an artificial intelligence (AI) agent for orchestrating a drug discovery workflow, in accordance with some example embodiments. Referring to FIG. 11E, the user interface (UI) 1140 is associated with a drug discovery workflow for designing a large molecule, such as a therapeutic protein or peptide. In some cases, the user interface (UI) 1140 may support interactions between, for example, one or more end users (e.g., the end user 310 in FIG. 3B) and the controller 110 to configure and execute a drug discovery workflow to design one or more large molecules. In some cases, the drug discovery workflow may be orchestrated by an artificial intelligence (AI) agent. In some cases, the user interface (UI) 1140 may be a part of a sequence of user interfaces (UIs) in a workflow to configure and execute the drug discovery workflow. As shown in FIG. 11E, in some cases, the user interface (UI) 1140 may include a home tab 1141, a design tab 1142, a humanize tab 1143, a deimmunize tab 1144, and a tools tab 1145. In some cases, the user interface (UI) 1400 may be generated, for example, by the controller 110, for display at the client device 160, when the home tab 1141 is selected. In some cases, the home tab 1141 may include a selection of experiments 1146 and projects 1147. In some cases, the one or more end users (e.g., the end user 310 in FIG. 3B) may invoke a drug discovery workflow by making a selection from the selection of experiments 1146 and/or projects 1147. In some cases, a single project may include one or more experiments.

FIG. 11F depicts a screenshot of another example of a user interface (UI) 1150 for configuring an artificial intelligence (AI) agent for orchestrating a drug discovery workflow, in accordance with some example embodiments. Referring to FIG. 11F, the user interface (UI) 1150 is associated with a drug discovery workflow for designing a large molecule, such as a therapeutic protein or peptide. In some cases, the user interface (UI) 1150 may support interactions between, for example, one or more end users (e.g., the end user 310 in FIG. 3B) and the controller 110 to configure and execute a drug discovery workflow to design one or more large molecules. In some cases, the drug discovery workflow may be orchestrated by an artificial intelligence (AI) agent. In some cases, the user interface (UI) 1150 may be a part of a sequence of user interfaces (UIs) in a workflow to configure and execute the drug discovery workflow. As shown in FIG. 11F, in some cases, the user interface (UI) 1150 may be generated, for example, by the controller 110, for display at the client device 160, when an existing experiment from the selection of experiments 1146 shown in the user interface (UI) 1140 is selected. In some cases, the user interface (UI) 1150 may receive, from the one or more end users, one or more inputs for configuring an experiment validating a molecule design. In some cases, the design tab 1142 may include a display of design details 1151, which may include an experiment name 1153, an experiment identifier 1154, a target molecule 1155, one or more seed molecules 1156, a project type 1157, a project name 1158, an antibody design format 1159, and a molecular identifier 1160. In some cases, the design tab 1142 may further include an order summary 1152 (e.g., with a total quantity of antibodies, unique light chains, unique heavy chains, kappa light chains, and lambda light chains) along with a submit for design product button 1161 such that the molecule design is synthesized for experimental validation. As noted, in some cases, the molecule design may be registered with a molecule registration system (e.g., the molecule registration system 140) and the unique molecular identifier assigned thereto may be associated with each synthesized batch of the molecule design.

FIG. 11G depicts a screenshot of another example of a user interface (UI) 1170 for configuring an artificial intelligence (AI) agent for orchestrating a drug discovery workflow, in accordance with some example embodiments. Referring to FIG. 11G, the user interface (UI) 1170 is associated with a drug discovery workflow for designing a large molecule, such as a therapeutic protein or peptide. In some cases, the user interface (UI) 1170 may support interactions between, for example, one or more end users (e.g., the end user 310 in FIG. 3B) and the controller 110 to configure and execute a drug discovery workflow to design one or more large molecules. In some cases, the drug discovery workflow may be orchestrated by an artificial intelligence (AI) agent. In some cases, the user interface (UI) 1170 may be a part of a sequence of user interfaces (UIs) in a workflow to configure and execute the drug discovery workflow. As shown in FIG. 11G, in some cases, the user interface (UI) 1170 may be generated, for example, by the controller 110, for display at the client device 160, when the one or more end users select the design tab 1142. In some cases, the user interface (UI) 1170 may support the configuration of a drug discovery workflow in which one or more antibody designs are created using an affinity maturation model and one or more corresponding properties of interest (e.g., expression yield, binding affinity, GRAF II, and iCAT) are predicted. In some cases, the user interface (UI) 1170 may include a target tab 1171 for selecting a target antigen and one or more seed molecules a parameterize tab 1174 for configuring the affinity maturation model, and a generate tab 1175 to initiate the design task. In some case, under the target tab 1171, the user interface (UI) 1170 may include a select a target menu 1172 (e.g., dropdown menu of a selection of target antigens) as well as an option to perform an advanced search, a selection of seed molecules 1173, and a graph 1176 displaying one or more properties of interest. In some cases, the example of the user interface (UI) 1170 shown in FIG. 11G may include the options to set X axis 1177 and set Y axis 1178 for configuring the properties of interest displayed (and compared) in the graph 1176.

FIG. 11H depicts a screenshot of another example of a user interface (UI) 1180 for configuring an artificial intelligence (AI) agent for orchestrating a drug discovery workflow, in accordance with some example embodiments. Referring to FIG. 11H, the user interface (UI) 1180 is associated with a drug discovery workflow for designing a large molecule, such as a therapeutic protein or peptide. In some cases, the user interface (UI) 1180 may support interactions between, for example, one or more end users (e.g., the end user 310 in FIG. 3B) and the controller 110 to configure and execute a drug discovery workflow to design one or more large molecules. In some cases, the drug discovery workflow may be orchestrated by an artificial intelligence (AI) agent. In some cases, the user interface (UI) 1180 may be a part of a sequence of user interfaces (UIs) in a workflow to configure and execute the drug discovery workflow. As shown in FIG. 11H, in some cases, the user interface (UI) 1180 may be generated, for example, by the controller 110, for display at the client device 160, when the one or more end users select the design tab 1142.

In some cases, the user interface (UI) 1180 may support the configuration of a drug discovery workflow in which one or more antibody designs are created using an affinity maturation model and one or more corresponding properties of interest (e.g., expression yield, binding affinity, GRAF II, and iCAT) are predicted. In some cases, the user interface (UI) 1180 may be generated in response to the selection of the parameterize tab 1174 for configuring the affinity maturation model. In some case, under the parameterize tab 1174, the user interface (UI) 1180 may include one or more input fields for configuring one or more corresponding sampling parameters 1181. For example, in FIG. 11H, the user interface (UI) 1180 may include input fields (e.g., text bar, dropdown menu, radial button, and/or the like) for configuring an affinity maturation method 1182, version 1183 (of the affinity maturation method), number of random seed molecules 1184, sampling by chain 1185, redesigned regions 1186, gap tokens 1187, guidance objectives 1188, and maximum edits 1189.

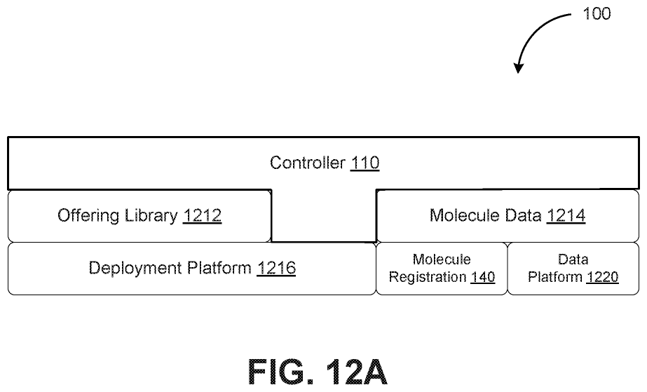

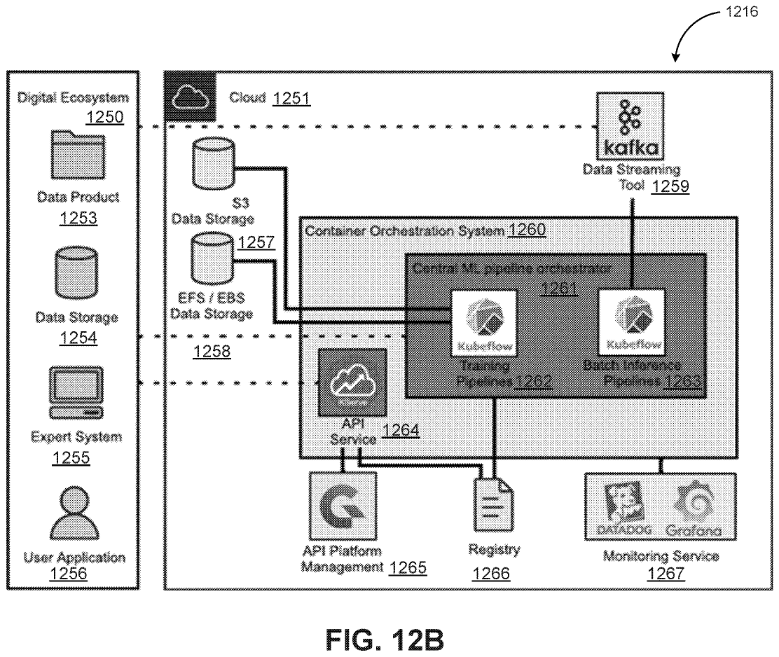

FIG. 12A depicts a schematic diagram illustrating the system design of an example of the drug discovery ecosystem 100, in accordance with some example embodiments. Referring to FIGS. 1 and 12A, in some cases, the drug discovery ecosystem 100 may include the controller 110, which supports the creation and configuration of drug discovery tools and artificial intelligence (AI) agents. For example, in some cases, the controller 110 of the drug discovery ecosystem 100 may interact with one or more builders (e.g., the builder 305 in FIG. 3A) to enable the contribution a reusable drug discovery tool, contribute a reusable capability to an existing drug discovery tool, consolidate one or more drug discovery workflows into an agent design, manage drug discovery tools and artificial intelligence (AI) agents, and/or the like. In some cases, the drug discovery ecosystem 100 may include an offering library 1212 exposing, to one or more end users of the drug discovery ecosystem 100 (e.g., the end user 310 in FIG. 3B), the drug discovery tools, artificial intelligence (AI) agents, and/or drug discovery workflows available from the drug discovery ecosystem 100. Examples of the offering library 1212 may include, for example, the drug discovery tool marketplace 400 shown in FIG. 4A, the artificial intelligence (AI) agent marketplace 450 shown in FIG. 4B, and/or the like. In some cases, the drug discovery ecosystem 100 may include the molecule registration system 140 and a data platform 1220 to manage the molecule data 1214 generated at the drug discovery ecosystem 100, for example, by executing one or more of the drug discovery tools, artificial intelligence (AI) agents, and/or drug discovery workflows included in the offering library 1212. For instance, in some cases, the molecule registration system 140 may provide centralized and uniform access to data associated with one or more molecule designs (or synthesized batches of molecule designs) generated at the drug discovery ecosystem 100 including, for example, chemical formulation, amino acid sequence, three-dimensional structure and/or substructure, properties, lineage, and/or the like. As described in more detail below, the drug discovery ecosystem 100 may include a deployment platform 1216 that allocates and scales computing resources to support the execution of the drug discovery tools, artificial intelligence (AI) agents, and/or drug discovery workflows included in the offering library 1212.

FIG. 12B depicts a schematic diagram illustrating the architecture of an example of the deployment platform 1216 in the system implementation of the drug discovery ecosystem 100 shown in FIG. 12A, in accordance with some example embodiments. In some example embodiments, the deployment platform 1216 may allocate and scale computing resources to support the execution of one or more artificial intelligence (AI) agents, drug discovery tools, and/or drug discovery workflows available from the drug discovery ecosystem 100. As shown in FIG. 12B, in some cases, the deployment platform 1216 may include a digital ecosystem 1252 coupled with cloud computing infrastructure 1251. As shown in FIG. 12B, in some cases, the digital ecosystem 1252 may include one or more of a data product 1253, a data storage 1254, an expert system 1255, and a user application 1256. In some cases, the cloud computing infrastructure 1251 may include a container orchestration system 1260 that includes a central machine learning (ML) pipeline orchestrator 1261. In some cases, the central machine learning (ML) pipeline orchestrator 1261 may orchestrate one or more training pipelines 1261, for example, utilizing training data stored in one or more of high performance and low latency block storage (e.g., Elastic Block Store (EBS)), scalable performance and low latency file storage (e.g., Elastic File System (EFS)), higher latency object storage (e.g., Simple Storage Service (S3)), and/or the like. In some cases, the central machine learning (ML) pipeline orchestrator 1261 may also orchestrate one or more inference pipelines 1263 using input data provided, for example, by a data streaming stool 1259. In some cases, the container orchestration system 1260 may be exposed via an application programming interface (API) service 1264 that is coupled with an application programming interface (API) platform management service 1265 and a registry 1266. In some cases, the container orchestration system 1260 may also be coupled with one or more monitoring services 1267 for monitoring one or more servers, databases, tools, and services associated with the drug discovery ecosystem 100.

In some example embodiments, infrastructure resources available from the cloud computing infrastructure 1251 may be managed and provisioned through an Infrastructure as Code (IaC) paradigm, which enables the creation, modification, and removal of infrastructure resources in a consistent and repeatable manner, reducing errors and improving efficiency. In some cases, under the Infrastructure as Code (IaC) paradigm, infrastructure changes may be well-documented and versioned, thereby facilitating traceability and auditability.

Referring again to FIG. 12B, in some cases, the cloud computing infrastructure 1251 may include the container orchestration system 1260 to meet the specific requirements of machine learning workflows, such as the various examples of drug discovery workflows described herein (e.g., the drug discovery workflow 800 shown in FIG. 8). In some cases, the container orchestration system 1260 may support the execution of both training (and retraining) pipelines 1262 and inference pipelines 1263, including by dynamic allocation of computing resources to enable the scaling of machine learning workflows in response to demand. In the context of the container orchestration system 1260, the jobs supporting the training (and retraining) pipelines 1262 may run over extended periods, with periodic traffic patterns. In some cases, the jobs associated with the training (and retraining) pipelines 1262 may check for new data availability at given frequencies to ensure that the corresponding drug discovery tools (e.g., computation models) are only trained when necessary. In some cases, one or more drug discovery tools may undergo non-parametric optimization in which, instead of incremental updates that can lead to catastrophic forgetting, the one or more drug discovery tools retrained using a continuously updated dataset. For example, in some cases, the jobs supporting the training (and retraining) pipelines 1262 may check for the presence of new data at the object storage 1257 and/or block and file storage 1258. In some cases, where the quantity of new data satisfies one or more thresholds, one or more corresponding drug discovery tools (e.g., computation models) may be retrained using the entire volume of data instead of just the new data that have accumulated since the previous update.

In some example embodiments, the inference pipelines 1263 may include both real-time (or online) inference workflows and batch (or offline) inference workflows. In some cases, the jobs supporting the real-time (or online) inference workflows jobs may be completed within a shorter time frame but with unpredictable traffic pattern necessitating computation resources capable of delivering low latency and high throughput. In some cases, the jobs supporting batch (or offline) inference jobs run for longer times with sporadic traffic patterns. It should be appreciated that the aforementioned pipelines (and workflows) may generally be computationally and/or memory-intensive. As such, the container orchestration system 1260 may support horizontal scaling (e.g., adding more machines or nodes) and vertical scaling (e.g., increasing the power of existing machines, such as adding more CPU, RAM, or storage) to accommodate these needs.

In some cases, a drug discovery workflow applying a sequence of drug discovery tools (e.g., molecule design computation models, property computation models, selection computation models, and/or the like) in the container orchestration system 1260 may be defined as one or more Directed Acyclic Graphs (DAGs), with each operation encapsulated in a software container (e.g., a Docker container). In this context, a “software container” or a “container” refers to an isolated and executable software package that bundles an application (e.g., an operation in a drug discovery workflow) and all its dependencies (e.g., code, runtime, libraries, configuration files, and/or the like) such that the application is able to run consistently across any computing environment. In some cases, this containerized approach may provide automation capabilities that minimize the risk of error and increase reproducibility. In some cases, containerization may also ensure isolation, reproducibility, and the ability to execute each operation in a drug discovery workflow with specific resource requirements. In some cases, the utilization of computation resources may be optimized by the container orchestration system 1260 tailoring resource allocation to the needs of each operation in the drug discovery workflow. For instance, simpler tasks (e.g., data schema validation) with lower memory needs and computational requirements may leverage cost-effective central processing unit (CPU) instances whereas more resource-intensive training tasks with substantial memory requirements may be allocated parallel graphics processing unit (GPU) instances. In some cases, the dynamic resource management and serverless scaling capabilities of the container orchestration system 1250, which allows computing resources to be adjusted based on demand, increase cost efficiency while ensuring that both the training (and retraining) pipelines 1262 and inference pipelines 1263 are able to seamlessly handle varying levels of load.

In some example embodiments, the data access requirements of the training pipelines 1262 and the batch inference pipelines 1263 are met by integration with data sources. In some cases, due to the complex data landscape in drug discovery, at least some of the data associated with the drug discovery ecosystem 100 may be maintained in temporary storage locations such as high performance and low latency block storage (e.g., Elastic Block Store (EBS)), scalable performance and low latency file storage (e.g., Elastic File System (EFS)), higher latency object storage (e.g., Simple Storage Service (S3)), and/or the like. In some cases, the temporary storage locations may host data such as pre-calculated features and large model binaries while keeping the data close to the computation. In some cases, this approach may minimize the traffic required to transfer data from on-premise infrastructure to the cloud computing infrastructure 1251 and vice versa, and therefore reduces cost and latency.

Table 1 below summarizes the components of the deployment platform 1216 depicted in FIGS. 12A-B.

TABLE 1

| Key | |
|---|---|
| Functionality | Description |
| Foundation & | Provides horizontal and vertical scalability for diverse, |
| Container | computationally intensive drug discovery workflows. |
| Orchestration | Adoption of a cloud-first approach resolved limitations of |
| | previous on-premises computing infrastructure. |
| ML Pipeline | Central orchestration for reliable, scalable training and |
| Orchestration | inference workflows (e.g., real-time and batch inference). |
| | Chosen for native containerization integration and |
| | flexibility for custom DAG-based workflows. |
| Real-time | Enables the serving of real-time (online) inference jobs |
| Model | with low-latency and auto-scaling capabilities. |
| Serving | |
| API | Manages REST APIs, ensuring standardized access, |
| Management | provides granular Role-Based Access Control (RBAC), |
| & Gateway | and allows for monitoring of API health. |

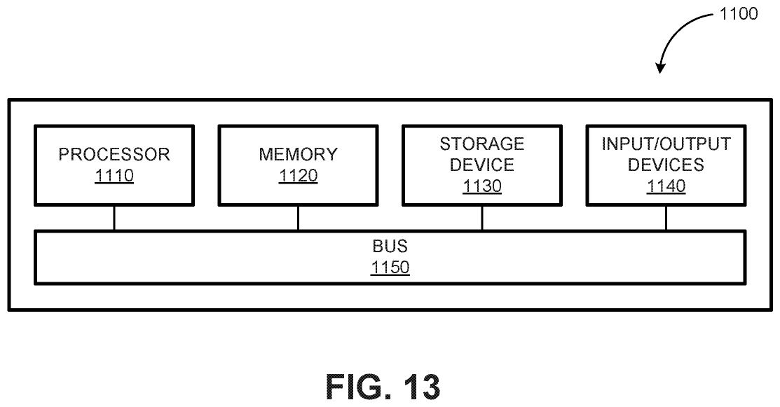

FIG. 13 depicts a block diagram illustrating an example of a computing system 1300, in accordance with some example embodiments. Referring to FIGS. 1-13, the computing system 1300 may be used to implement at least a portion of the drug discovery ecosystem 100 shown in FIG. 1 including, for example, the controller 110, the one or more agents 120, the tool collection 130, the molecule registration system 140, the laboratory equipment 150, the client device 160, and/or any components therein.

As shown in FIG. 13, the computing system 1300 can include a processor 1310, a memory 1320, a storage device 1330, and input/output devices 1340. The processor 1310, the memory 1320, the storage device 1330, and the input/output devices 1340 can be interconnected via a system bus 1350. The processor 1310 is capable of processing instructions for execution within the computing system 1300. Such executed instructions can implement one or more components of, for example, the controller 110, the one or more agents 120, the tool collection 130, the molecule registration system 140, the laboratory equipment 150, the client device 160, and/or the like. In some example embodiments, the processor 1310 can be a single-threaded processor. Alternately, the processor 1310 can be a multi-threaded processor. The processor 1310 is capable of processing instructions stored in the memory 1320 and/or on the storage device 1330 to display graphical information for a user interface provided via the input/output device 1340.

The memory 1320 is a computer readable medium such as volatile or non-volatile that stores information within the computing system 1300. The memory 1320 can store data structures representing configuration object databases, for example. The storage device 1330 is capable of providing persistent storage for the computing system 1300. The storage device 1330 can be a floppy disk device, a hard disk device, an optical disk device, or a tape device, or other suitable persistent storage means. The input/output device 1340 provides input/output operations for the computing system 1300. In some example embodiments, the input/output device 1340 includes a keyboard and/or pointing device. In various implementations, the input/output device 1340 includes a display unit for displaying graphical user interfaces.

According to some example embodiments, the input/output device 1340 can provide input/output operations for a network device. For example, the input/output device 1340 can include Ethernet ports or other networking ports to communicate with one or more wired and/or wireless networks (e.g., a local area network (LAN), a wide area network (WAN), the Internet).

In some example embodiments, the computing system 1300 can be used to execute various interactive computer software applications that can be used for organization, analysis and/or storage of data in various formats. Alternatively, the computing system 1300 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1340. The user interface can be generated and presented to a user by the computing system 1300 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT), a liquid crystal display (LCD), a light emitting diode (LED) monitor, or an organic light emitting diode (OLED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1-84. (canceled)

85. A non-transitory computer readable medium storing instruction, which when executed by at least one data processor, result in operations comprising:
receiving, by an artificial intelligence (AI) agent, a query specifying an objective;
in response to receiving the query, executing an artificial intelligence (AI) agent, wherein the artificial intelligence (AI) agent orchestrates a drug discovery workflow for accomplishing the objective specified by the query, and wherein the artificial intelligence (AI) agent orchestrates the drug discovery workflow by at least
selecting, based at least on the query, a drug discovery tool,
generating, based at least on a input of the drug discovery tool, a prompt soliciting the input of the drug discovery tool, and
upon receiving the input of the drug discovery tool, applying the drug discovery tool with the input.

86. A system, comprising:
at least one data processor; and
at least one memory storing instructions, which when executed by the at least one data processor, result in operations comprising:
receiving, by an artificial intelligence (AI) agent, a query specifying an objective;
in response to receiving the query, executing an artificial intelligence (AI) agent, wherein the artificial intelligence (AI) agent orchestrates a drug discovery workflow for accomplishing the objective specified by the query, and wherein the artificial intelligence (AI) agent orchestrates the drug discovery workflow by at least
selecting, based at least on the query, a drug discovery tool,
generating, based at least on a input of the drug discovery tool, a prompt soliciting the input of the drug discovery tool, and
upon receiving the input of the drug discovery tool, applying the drug discovery tool with the input.

87. The system of claim 86, wherein the query comprises a natural language query.

88. The system of claim 86, wherein the drug discovery tool comprises a molecule design computation model trained to generate one or more molecule designs.

89. The system of claim 86, wherein the drug discovery tool comprises a selection computation model trained to rank two or more molecule designs based on one or more properties of interest.

90. The system of claim 86, wherein the drug discovery tool comprises a property computation model trained to determine one or more properties of interest present in a molecule design.

91. The system of claim 86, wherein the drug discovery tool comprises a structure computation model trained to determine a three-dimensional structure of a molecule design.

92. The system of claim 86, wherein the drug discovery tool comprises a docking computation model trained to determine a three-dimensional structure of a molecule design in complex with one or more other molecules.

93. The system of claim 86, wherein the drug discovery tool comprises a metabolite computation model trained to determine one or more metabolic transformations of a molecule design.

94. The system of claim 86, wherein the artificial intelligence (AI) agent further orchestrates the drug discovery workflow by at least generating a user interface (UI) displaying at least a portion of an output of the drug discovery tool.

95. The system of claim 86, wherein the artificial intelligence (AI) agent further orchestrates the drug discovery workflow by at least
upon applying the drug discovery tool, identifying a subsequent drug discovery tool for accomplishing the objective specified by the query, and
applying the subsequent drug discovery tool.

96. The system of claim 95, wherein the artificial intelligence (AI) agent identifies the subsequent drug discovery tool based on at least one of (i) the objective specified by the query, (ii) an output of the drug discovery tool, and (iii) one or more subsequent inputs received from an end user.

97. The system of claim 95, wherein the artificial intelligence (AI) agent applies the subsequent drug discovery tool reusing an input to and/or an output from another drug discovery tool in the drug discovery workflow.

98. The system of claim 95, wherein the artificial intelligence (AI) agent generates an additional prompt soliciting a input of the subsequent drug discovery tool.

99. The system of claim 86, wherein the artificial intelligence (AI) agent orchestrates the drug discovery workflow by at least identifying, from a plurality of available drug discovery tools, a subset of drug discovery tools for accomplishing the objective.

100. The system of claim 86, wherein the artificial intelligence (AI) agent orchestrates the drug discovery workflow by at least applying a plurality of drug discovery tools in accordance with a predetermined sequence.

101. The system of claim 86, wherein the artificial intelligence (AI) agent orchestrates the drug discovery workflow by at least dynamically determining a sequence in which to apply a plurality of drug discovery tools.

102. The system of claim 86, wherein at least a portion of the query comprise a selection of one or more preconfigured inputs provided to an end user.

103. The system of claim 86, wherein the objective comprises identifying a molecule exhibiting one or more properties of interest.

104. The system of claim 103, wherein the molecule comprises a therapeutic protein, a therapeutic peptide, a small molecule drug compound, a multispecific antibody, or a bioconjugate.

105. The system of claim 103, wherein the one or more properties of interest include at least one of binding affinity, binding specificity, biological activity, and developability.

106. The system of claim 86, wherein the artificial intelligence (AI) agent selects the drug discovery tool by at least
synthesizing a set of reference intention-action pairs,
identifying, within the set of reference intention-action pairs, one or more intentions that are a semantic match to the objective specified by the query,
determining a trajectory of actions based at least on one or more intention-action pairs corresponding to the one or more intentions that are a semantic match to the objective specified by the query, and
determining, based at least on the trajectory actions, the drug discovery tool as a part of a sequence of drug discovery tools for accomplishing the objective.

107. The system of claim 106, wherein the synthesizing the set of reference intention-action pairs includes
determining, based at least on a set of available drug discovery tools, a set of valid action chains in which an upstream drug discovery tool's output datatype corresponds to a downstream drug discovery tool's input datatype,
applying a language model to determine, for each valid action chain, a corresponding intention, and
generating each intention-action pair in the set of reference intention-action pairs to include a valid action chain and the corresponding intention.

108. The system of claim 86, wherein the artificial intelligence (AI) agent selects the drug discovery tool by at least determining one or more benchmarks indicative of a performance of the drug discovery tool relative to one or more other drug discovery tools available to perform a same task.

109. The system of claim 86, wherein the artificial intelligence (AI) agent orchestrates the drug discovery workflow by at least routing an agent flow comprising one or more specialized artificial intelligence (AI) agents.

110. The system of claim 109, wherein each specialized artificial intelligence (AI) agent orchestrates an application of a corresponding drug discovery tool comprising the drug discovery workflow.

111. The system of claim 109, wherein a specialized artificial intelligence (AI) agent routes the agent flow back to the artificial intelligence (AI) agent upon determining that a drug discovery tool associated with the specialized artificial intelligence (AI) agent has completed one or more corresponding tasks.

112. The system of claim 109, wherein a specialized artificial intelligence (AI) agent routes the agent flow back to the artificial intelligence (AI) agent upon determining that a task assigned to the specialized artificial intelligence (AI) agent exceeds a scope of a drug discovery tool associated with the specialized artificial intelligence (AI) agent.

113. The system of claim 109, wherein the executing the artificial intelligence (AI) agent includes adding, to one or more conversation turns between an end user invoking the drug discovery workflow and the artificial intelligence (AI) agent, one or more nodes guiding the artificial intelligence (AI) agent to verify a critical action, follow a sequence of the drug discovery workflow, and/or handle hallucinated values generated by one or more drug discovery tools.

114. A computer-implemented method, comprising:
receiving, by an artificial intelligence (AI) agent, a query specifying an objective;
in response to receiving the query, executing an artificial intelligence (AI) agent, wherein the artificial intelligence (AI) agent orchestrates a drug discovery workflow for accomplishing the objective specified by the query, and wherein the artificial intelligence (AI) agent orchestrates the drug discovery workflow by at least
selecting, based at least on the query, a drug discovery tool,
generating, based at least on a input of the drug discovery tool, a prompt soliciting the input of the drug discovery tool, and
upon receiving the input of the drug discovery tool, applying the drug discovery tool with the input.
